(19) United States
(12) Patent Application Publication
(10) Pub. No.: US 2021/0066617 A1
(43) Pub. Date: Mar. 4, 2021

(54) LIGHT-EMITTING DEVICE AND ELECTRONIC APPARATUS INCLUDING THE SAME

(71) Applicant: Samsung Display Co., Ltd., Yongin-si (KR)

(72) Inventors: Hyun SHIN, Yongin-si (KR); Jinwon SUN, Yongin-si (KR)

(21) Appl. No.: 17/003,743

(22) Filed: Aug. 26, 2020

(30) Foreign Application Priority Data
Aug. 30, 2019 (KR) 10-2019-0107650
Jul. 22, 2020 (KR) 10-2020-0091260

(51) Int. Cl. H01L 51/00; C07D 209/86; C09K 11/06; C07D 209/88; C07F 7/08; C07D 409/14; C07F 15/00

(52) U.S. Cl. CPC H01L 51/0072; H01L 51/5016; C09K 11/06; C07D 209/88; C07F 7/0812; H01L 51/0094; C07D 409/14; H01L 51/0067; H01L 51/0074; C07F 15/0086; H01L 51/0087; C09K 2211/1018; C09K 2211/1044; C09K 2211/185; H01L 2251/5384; H01L 2251/552; C07D 209/86

(57) ABSTRACT

A light-emitting device includes a first electrode, a second electrode, and an interlayer between the first electrode and the second electrode and including an emission layer, wherein the emission layer includes a first host, a second host, and a dopant, the first host and the second host are to form an exciplex, and the exciplex, the first host, and the second host satisfy Condition 1:

0.5 eV≤[{T₁(H1)−S₁(Ex)}+{T₁(H2)−S₁(Ex)}]≤0.9 eV Condition 1

wherein, in Condition 1, T₁(H1) indicates a lowest excitation triplet energy level of the first host, T₁(H2) indicates a lowest excitation triplet energy level of the second host, and S₁(Ex) indicates a lowest excitation singlet energy level of the exciplex.

### CROSS-REFERENCE TO RELATED APPLICATION

This application claims priority to and the benefit of Korean Patent Application No. 10-2019-0107650, filed on Aug. 30, 2019, and Korean Patent Application No. 10-2020-0091260, filed on Jul. 22, 2020, each in the Korean Intellectual Property Office, the disclosures of both of which are incorporated herein in their entireties by reference.

### BACKGROUND

### 1. Field

One or more embodiments relate to a light-emitting device and an electronic apparatus including the same.

### 2. Description of Related Art

Light-emitting devices are self-emission devices that produce full-color images, and also have wide viewing angles, high contrast ratios, short response times, as well as excellent characteristics in terms of brightness, driving voltage, and/or response speed.

In a light-emitting device, a first electrode is on a substrate, and a hole transport region, an emission layer, an electron transport region, and a second electrode are sequentially formed on the first electrode. Holes provided from the first electrode may move toward the emission layer through the hole transport region, and electrons provided from the second electrode may move toward the emission layer through the electron transport region. Carriers, such as holes and electrons, recombine in the emission layer to produce excitons. These excitons transition from an excited state to a ground state to thereby generate light.

### SUMMARY

Aspects according to one or more embodiments are directed toward a light-emitting device and an electronic apparatus including the same.

Additional aspects will be set forth in part in the description which follows and, in part, will be apparent from the description, or may be learned by practice of the presented embodiments of the disclosure.

According to one or more embodiments, a light-emitting device includes a first electrode, a second electrode, and an interlayer between the first electrode and the second electrode and including an emission layer,

wherein the emission layer includes a first host, a second host, and a dopant,

the first host and the second host are to form an exciplex, and

the exciplex, the first host, and the second host satisfy Condition 1:

0.5 eV≤[{T₁(H1)−S₁(Ex)}+{T₁(H2)−S₁(Ex)}]0.9 eV Condition 1

wherein, in Condition 1,

T₁(H1) indicates a lowest excitation triplet energy level of the first host,

T₁(H2) indicates a lowest excitation triplet energy level of the second host, and

S₁(Ex) indicates a lowest excitation singlet energy level of the exciplex.

According to one or more embodiments, an electronic apparatus includes the light-emitting device and a thin-film transistor, wherein the thin-film transistor includes a source electrode and a drain electrode, and the first electrode of the light-emitting device is electrically connected to the source electrode or the drain electrode of the thin-film transistor.

### BRIEF DESCRIPTION OF THE DRAWINGS

The above and other aspects, features, and enhancements of certain embodiments of the disclosure will be more apparent from the following description taken in conjunction with the accompanying drawings, in which:

FIG. 1 is a schematic view of a light-emitting device according to an embodiment;

FIG. 2 is a schematic view of a light-emitting device according to another embodiment; and

FIG. 3 is a schematic view of a light-emitting device according to another embodiment.

### DETAILED DESCRIPTION

Reference will now be made in more detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

Because the disclosure may have diversely modified embodiments, preferred embodiments are illustrated in the drawings and are described in the detailed description. An effect and a characteristic of the disclosure, and a method of accomplishing these will be apparent when referring to embodiments described with reference to the drawings. The disclosure may, however, be embodied in many different forms and should not be construed as limited to the embodiments set forth herein.

One or more embodiments of the disclosure will be described below in more detail with reference to the accompanying drawings. The components that are the same or are in correspondence with each other are rendered the same reference numeral regardless of the figure number, and redundant explanations are omitted.

An expression used in the singular encompasses the expression of the plural, unless it has a clearly different meaning in the context.

It will be further understood that the terms “comprises” and/or “comprising” as used herein specify the presence of stated features or elements, but do not preclude the presence or addition of one or more other features or elements.

It will be understood that when a layer, region, or element is referred to as being “formed on” another layer, area, or element, it can be directly or indirectly formed on the other layer, region, or element. That is, for example, intervening layers, regions, or elements may be present.

Sizes of elements in the drawings may be exaggerated for convenience of explanation. In other words, because sizes and thicknesses of components in the drawings are arbitrarily illustrated for convenience of explanation, the following embodiments are not limited thereto.

The term “interlayer” as used herein refers to a single layer and/or a plurality of layers between a first electrode and a second electrode of a light-emitting device.

According to one or more embodiments, a light-emitting device includes: a first electrode; a second electrode; and an interlayer located between the first electrode and the second electrode and including an emission layer,

wherein the emission layer includes a first host, a second host, and a dopant,

the first host and the second host are configured to form an exciplex (e.g., the first host and the second host form an exciplex), and

the exciplex, the first host, and the second host satisfy Condition 1:

0.5 eV≤[{T₁(H1)−S₁(Ex)}+{T₁(H2)−S₁(Ex)}]0.9 eV Condition 1

wherein, in Condition 1,

T₁(H1) indicates a lowest excitation triplet energy level of the first host,

T₁(H2) indicates a lowest excitation triplet energy level of the second host, and

S₁(Ex) indicates a lowest excitation singlet energy level of the exciplex.

In more detail, T₁(H1) indicates a lowest excitation triplet energy level of the first host at an onset wavelength in a photoluminescence (PL) spectrum, T₁(H2) indicates a lowest excitation triplet energy level of the second host at an onset wavelength in a PL spectrum, and S₁(Ex) indicates a lowest excitation singlet energy level of the exciplex at an onset wavelength in a PL spectrum.

The expression “lowest excitation singlet energy level at an onset wavelength” as used herein refers to a singlet energy level at a starting point of a PL spectrum. The lowest excitation singlet energy level may be calculated from singlet energy level at a point (i.e., an x-intercept) that meets a wavelength axis of a function obtained by plotting the PL spectrum as a quadratic function.

The expression “lowest excitation triplet energy level at an onset wavelength” as used herein refers to a triplet energy level at a starting point of a PL spectrum. The lowest excitation triplet energy level may be calculated from triplet energy level at a point (i.e., an x-intercept) that meets a wavelength axis of a function obtained by plotting the PL spectrum as a quadratic function.

Here, the PL spectrum at room temperature is measured by utilizing a PL measuring device at room temperature after dissolving a compound in toluene at a concentration of 1×10⁻⁵ M, and the PL spectrum at a low temperature is measured at a low temperature (77 K) after dissolving a compound in TFT at a concentration of 1×10⁻⁵ M. As compared with the PL spectrum at room temperature, only peaks observed only at the low temperature are analyzed, so as to derive a singlet energy level and a triplet energy level.

Hereinafter, the expression “[{T₁(H1)−S₁(Ex)}+{T₁(H2)−S₁(Ex)}]” is referred to as a triplet confinement factor (TCF).

Because the light-emitting device satisfies Condition 1, excitons generated in the exciplex may move to the first host and/or the second host, and in this regard, deterioration of the light-emitting device caused by excitons generated in the first host and/or the second host may be suppressed.

When a TCF value is equal to or greater than 0.5 eV, it is difficult (e.g., substantially impossible) for the excitons generated in the exciplex to transfer to a triplet level of the first host and/or the second host, and accordingly, an unstable excitation state of the first host and/or the second host should not exist. Thus, in this case, the stability of the emission layer may be improved, thereby improving a lifespan of the light-emitting device.

When a TCF value is equal to or less than 0.9 eV, an exciplex having an energy level sufficient to transfer the exciton energy to a dopant suitable for blue luminescence may be formed.

In an embodiment, the lowest excitation triplet energy level of the exciplex may be greater than 2.7 eV. When the lowest excitation triplet energy level of the exciplex is within the range above, the light-emitting device may be suitable for blue luminescence.

The first host may have a carbazole partial structure. That is, the first host may include a tricyclic structure in which two rings are fused on either side of a nitrogen containing five membered ring.

In an embodiment, the first host may be represented by Formula 1:

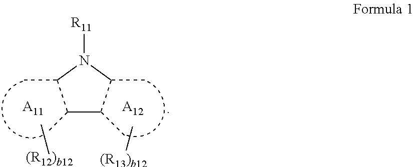

In Formula 1,

A₁₁ and A₁₂ may each independently be a C₃-C₂₀ carbocyclic group or a C₁-C₂₀ heterocyclic group,

R₁₁ to R₁₃ may each independently be a group represented by *-(L₁₁)ₐ₁₁-R₁₄, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂), wherein at least one of R₁₁ to R₁₃ may be a group represented by *-(L₁₁)ₐ₁₁-R₁₄,

b12 and b13 may each independently be an integer from 1 to 10,

L₁₁ may be a C₃-C₆₀ carbocyclic group unsubstituted or substituted with at least one R₁₀ₐ or a C₁-C₆₀ heterocyclic group unsubstituted or substituted with at least one R₁₀ₐ,

a11 may be an integer from 0 to 5,

R₁₄ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

Q₃₀₁ to Q₃₀₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆₀ heterocyclic group that is unsubstituted or substituted with deuterium, —F, a cyano group, a C₁-C₆₀ alkyl group, a C₁-C₆₀ alkoxy group, a phenyl group, a biphenyl group, or any combination thereof,

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁⁻C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

wherein Q₁₁ to Q₁₃, Q₂₁ to Q₂₃, Q₃₁ to Q₃₃ and Q₃₀₁ to Q₃₀₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a alkoxy group, or a C₃-C₆₀ carbocyclic group or a C₁-C₆₀ heterocyclic group that is unsubstituted or substituted with deuterium, —F, a cyano group, a C₁-C₆₀ alkyl group, a C₁-C₆₀ alkoxy group, a phenyl group, a biphenyl group, or any combination thereof, and

* indicates a binding site to a neighboring atom.

In an embodiment, the first host may be selected from Group I:

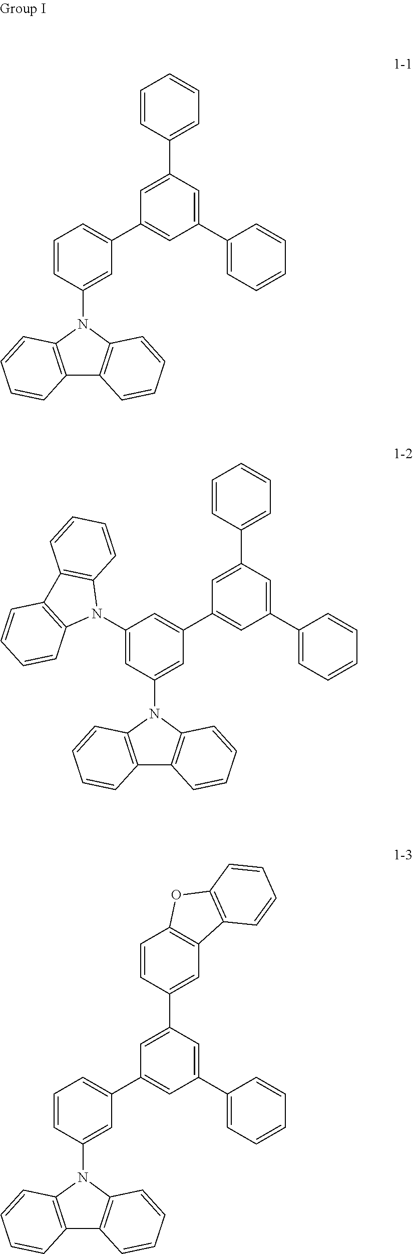 [SMILES: c1ccc(-c2cc(-c3ccccc3)cc(-c3cccc(-n4c5ccccc5c5ccccc54)c3)c2)cc1 ; c1ccc(-c2cc(-c3ccccc3)cc(-c3cc(-n4c5ccccc5c5ccccc54)cc(-n4c5ccccc5c5ccccc54)c3)c2)cc1 ; c1ccc(-c2cc(-c3cccc(-n4c5ccccc5c5ccccc54)c3)cc(-c3ccc4oc5ccccc5c4c3)c2)cc1]

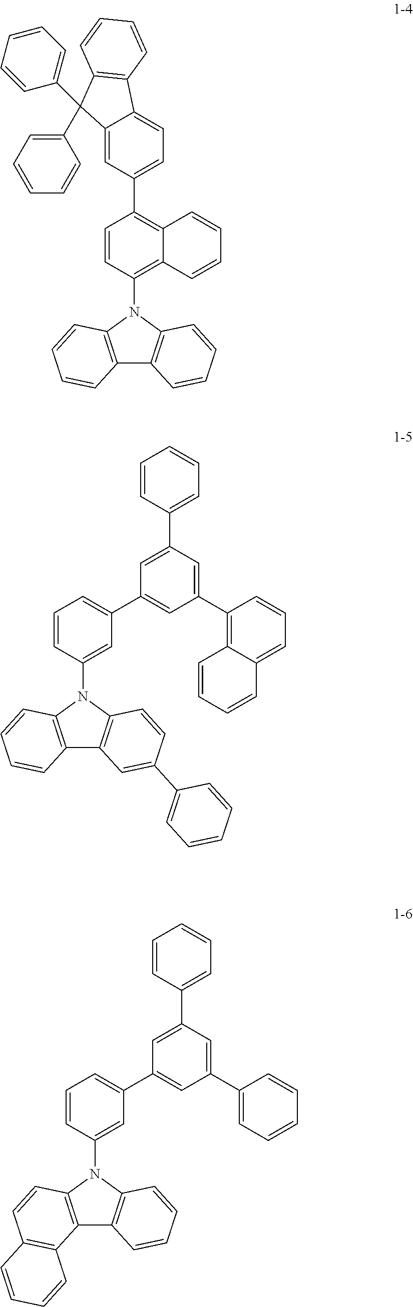 [SMILES: c1ccc(C2(c3ccccc3)c3ccccc3-c3ccc(-c4ccc(-n5c6ccccc6c6ccccc65)c5ccccc45)cc32)cc1 ; c1ccc(-c2cc(-c3cccc(-n4c5ccccc5c5cc(-c6ccccc6)ccc54)c3)cc(-c3cccc4ccccc34)c2)cc1 ; c1ccc(-c2cc(-c3ccccc3)cc(-c3cccc(-n4c5ccccc5c5c6ccccc6ccc54)c3)c2)cc1]

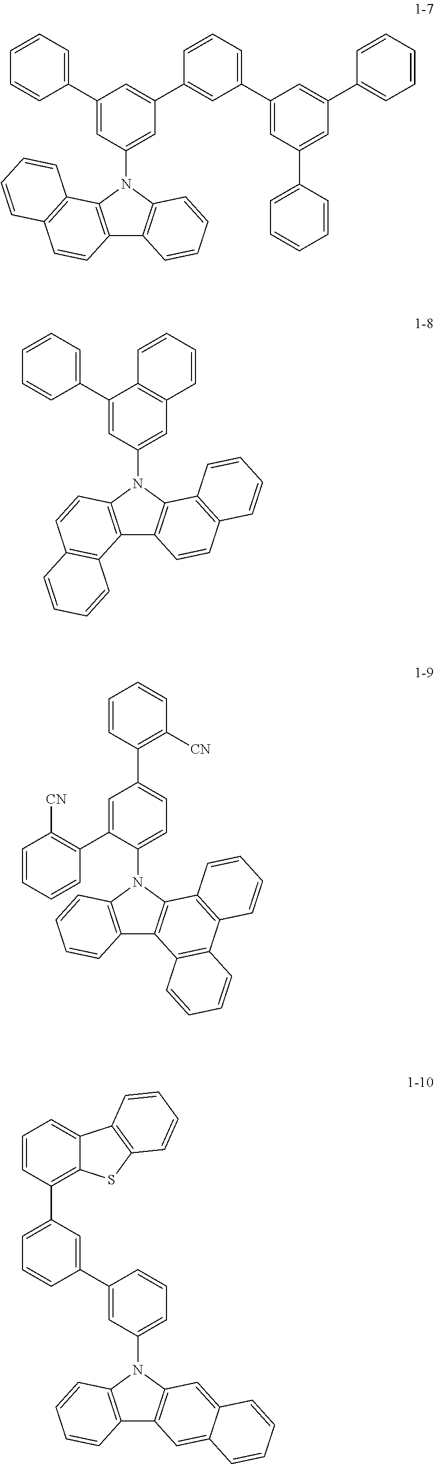 [SMILES: c1ccc(-c2cc(-c3ccccc3)cc(-c3cccc(-c4cc(-c5ccccc5)cc(-n5c6ccccc6c6ccc7ccccc7c65)c4)c3)c2)cc1 ; c1ccc(-c2cc(-n3c4ccc5ccccc5c4c4ccc5ccccc5c43)cc3ccccc23)cc1 ; N#Cc1ccccc1-c1ccc(-n2c3ccccc3c3c4ccccc4c4ccccc4c32)c(-c2ccccc2C#N)c1 ; c1cc(-c2cccc(-n3c4ccccc4c4cc5ccccc5cc43)c2)cc(-c2cccc3c2sc2ccccc23)c1]

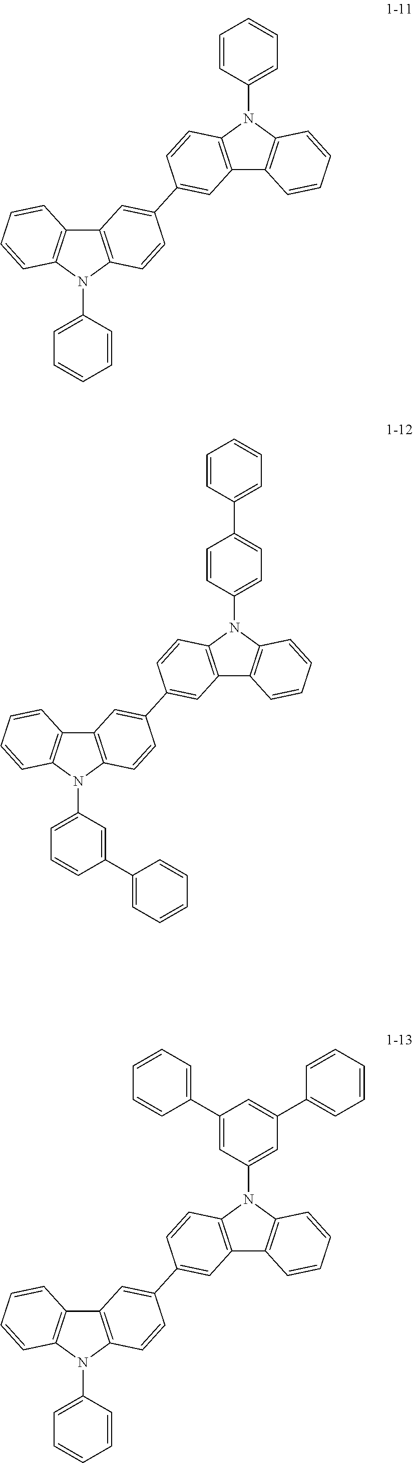 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

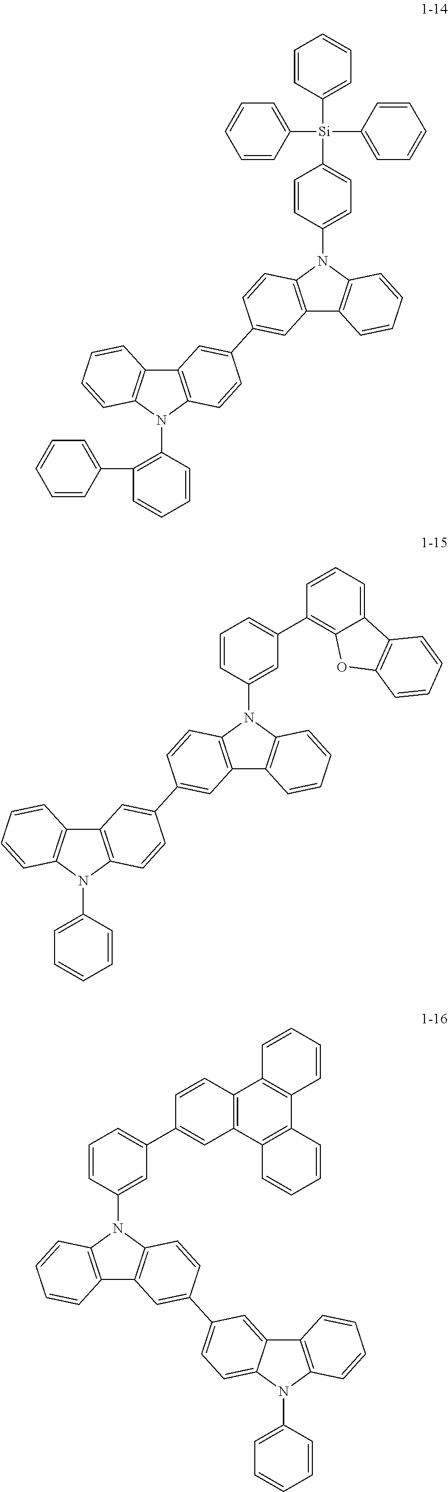 [SMILES: c1ccc(-c2ccccc2-n2c3ccccc3c3cc(-c4ccc5c(c4)c4ccccc4n5-c4ccc([Si](c5ccccc5)(c5ccccc5)c5ccccc5)cc4)ccc32)cc1 ; c1ccc(-n2c3ccccc3c3cc(-c4ccc5c(c4)c4ccccc4n5-c4cccc(-c5cccc6c5oc5ccccc56)c4)ccc32)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1]

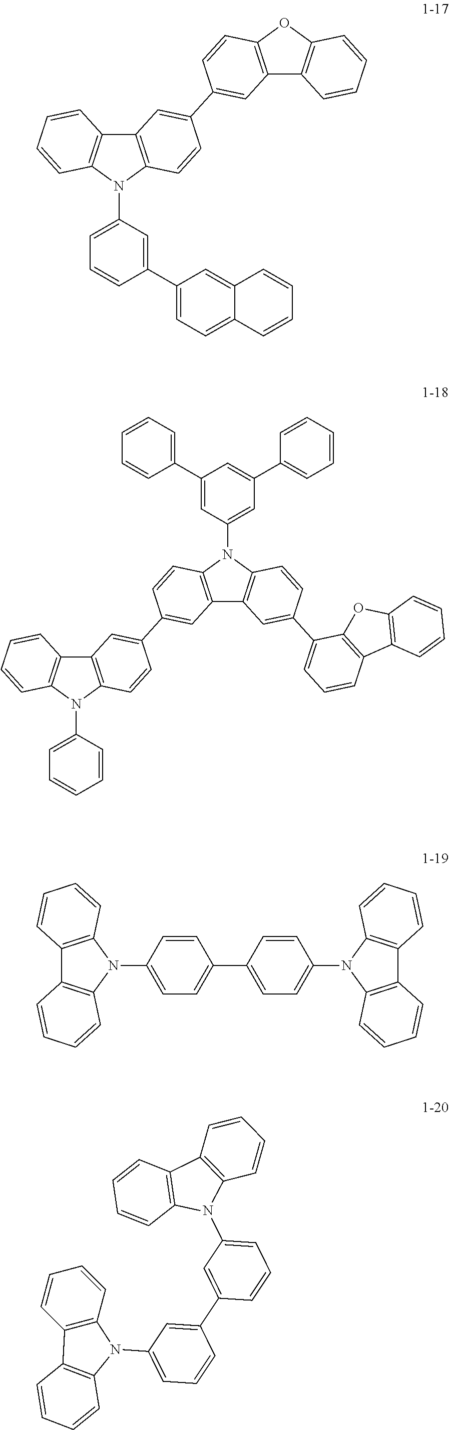 [SMILES: c1cc(-c2ccc3ccccc3c2)cc(-n2c3ccccc3c3cc(-c4ccc5oc6ccccc6c5c4)ccc32)c1 ; c1ccc(-c2cc(-c3ccccc3)cc(-n3c4ccc(-c5ccc6c(c5)c5ccccc5n6-c5ccccc5)cc4c4cc(-c5cccc6c5oc5ccccc56)ccc43)c2)cc1 ; c1ccc2c(c1)c1ccccc1n2-c1ccc(-c2ccc(-n3c4ccccc4c4ccccc43)cc2)cc1 ; c1cc(-c2cccc(-n3c4ccccc4c4ccccc43)c2)cc(-n2c3ccccc3c3ccccc32)c1]

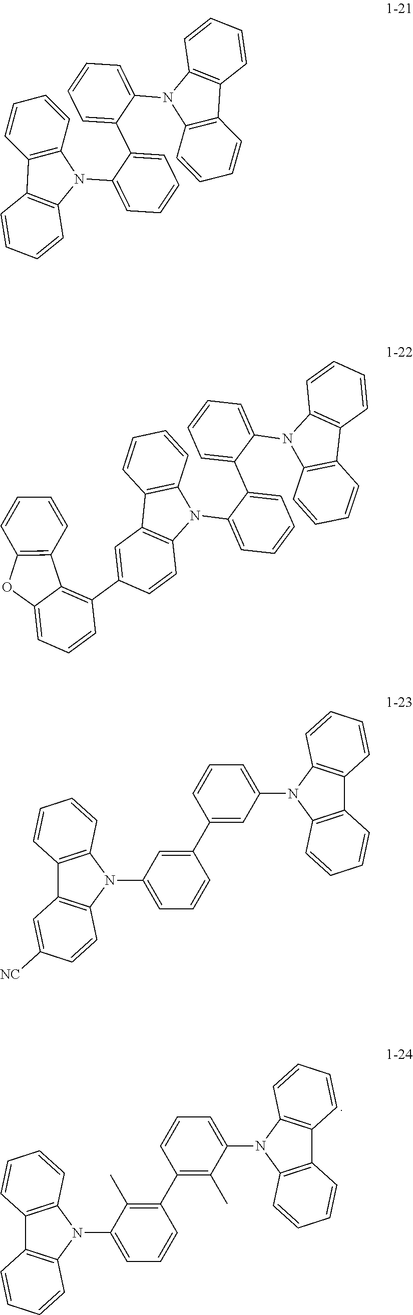 [SMILES: c1ccc(-n2c3ccccc3c3ccccc32)c(-c2ccccc2-n2c3ccccc3c3ccccc32)c1 ; c1ccc(-n2c3ccccc3c3ccccc32)c(-c2ccccc2-n2c3ccccc3c3cc(-c4cccc5oc6ccccc6c45)ccc32)c1 ; N#Cc1ccc2c(c1)c1ccccc1n2-c1cccc(-c2cccc(-n3c4ccccc4c4ccccc43)c2)c1 ; Cc1c(-c2cccc(-n3c4ccccc4c4ccccc43)c2C)cccc1-n1c2ccccc2c2ccccc21]

The second host may have a triazine partial structure. That is, the second host may include a triazine group in the structure.

In an embodiment, the second host may be represented by Formula 2:

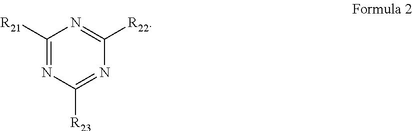

In Formula 2,

R₂₁ to R₂₃ may each independently be a group represented by *-(L₂₁)ₐ₂₁-R₂₄, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂), wherein at least one of R₂₁ to R₂₃ may be a group represented by *-(L₂₁)ₐ₂₁-R₂₄,

L₂₁ may be a C₃-C₆₀ carbocyclic group unsubstituted or substituted with at least one R₁₀ₐ or a C₁-C₆₀ heterocyclic group unsubstituted or substituted with at least one R₁₀ₐ,

a21 may be an integer from 0 to 5,

R₂₄ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

Q₃₀₁ to Q₃₀₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆₀ heterocyclic group that is unsubstituted or substituted with deuterium, —F, a cyano group, a C₁-C₆₀ alkyl group, a C₁-C₆₀ alkoxy group, a phenyl group, a biphenyl group, or any combination thereof, and

* indicates a binding site to a neighboring atom.

In an embodiment, the second host may be selected from Group II (i.e., selected from compounds 2-1 to 2-13 in Group II):

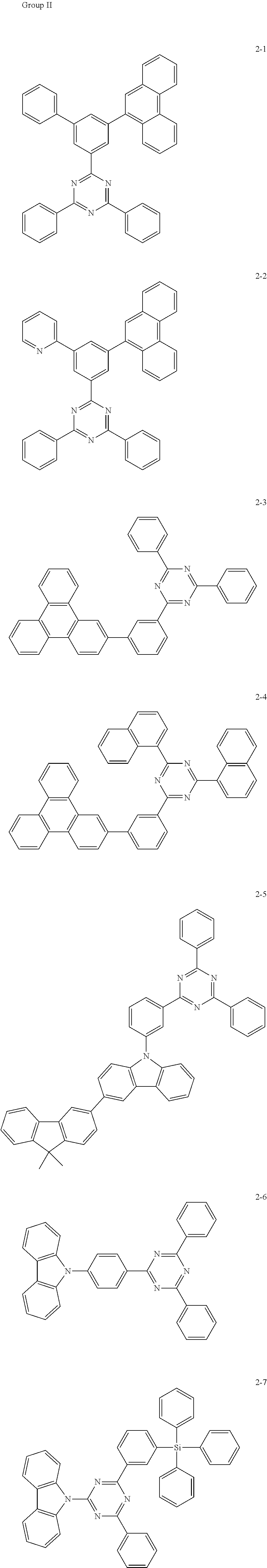

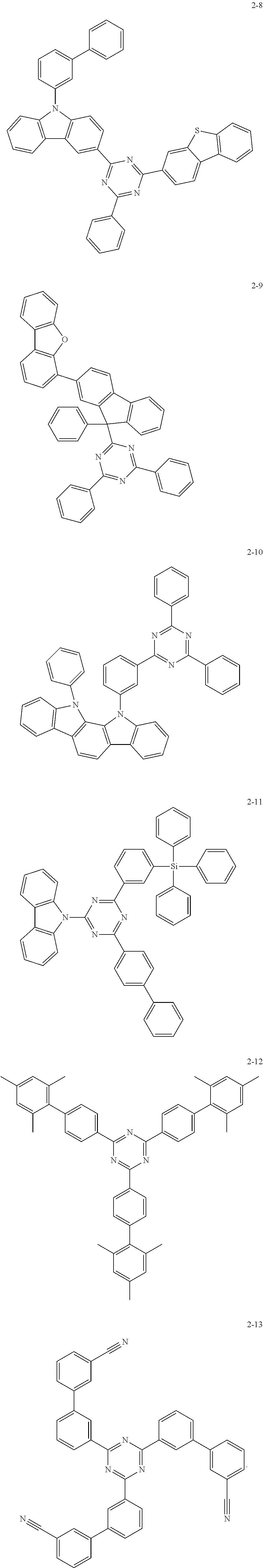

The first host and the second host may not substantially emit light. That is, The first host and the second host may not emit any substantial amount of light.

In general, when electrons provided from an electron transport region are not efficiently injected to an emission layer, charges are accumulated at an interface between the emission layer and the electron transport region, resulting in deterioration of the interface. Similarly, when holes provided from a hole transport region are not efficiently injected to an emission layer, charges are accumulated at an interface between the emission layer and the hole transport region, resulting in deterioration of the interface. Consequently, the light-emitting device may have a reduced lifespan.

Because the second host is a compound including (essentially including) an electron transport moiety, the second host may be utilized (e.g., easily utilized) to adjust electron transport characteristics of the light-emitting device. Because the first host is a compound not including an electron transport moiety, the first host may be utilized (e.g., easily utilized) to adjust hole transport characteristics of the light-emitting device. Accordingly, the light-emitting device may have an optimized or desired charge balance in the emission layer.

The dopant may emit phosphorescence, fluorescence, or delayed fluorescence.

For example, the dopant may emit blue light, and in more detail, may have a maximum luminescence wavelength in a range of about 420 nm to about 490 nm. However, embodiments of the present disclosure are not limited thereto.

In the emission layer, an amount of the first host may be in a range of about 10 wt % to about 90 wt % based on the total weight of the emission layer.

In the emission layer, an amount of the second host may be in a range of about 10 wt % to about 90 wt % based on the total weight of the emission layer.

In the emission layer, an amount of the dopant may be in a range of about 0.25 wt % to about 5 wt % based on the total weight of the emission layer.

In an embodiment, the first electrode may be an anode, the second electrode may be a cathode, and the interlayer may further include a hole transport region between the first electrode and the emission layer and an electron transport region between the second electrode and the emission layer.

In an embodiment, the hole transport region may further include a first layer, and the first layer may include (e.g., consist of) an organometallic compound.

In an embodiment, the electron transport region may further include a second layer, and the second layer may include (e.g., consist of) an organometallic compound.

In an embodiment, the hole transport region may further include a first layer, and the electron transport region may further include a second layer, wherein the first layer and the second layer may each independently include (e.g., consist of) an organometallic compound.

In an embodiment, the organometallic compound may be represented by Formula 3:

M₃₁(L₃₁)ₙ₃₁(L₃₂)ₙ₃₂ Formula 3

wherein, in Formula 3,

M₃₁ may be platinum (Pt) or iridium (Ir),

L₃₁ may be a ligand represented by one of Formulae 3A to 3D,

n31 may be 1 or 2,

L₃₂ may be an organic ligand, and

n32 may be 0, 1, 2, 3, or 4,

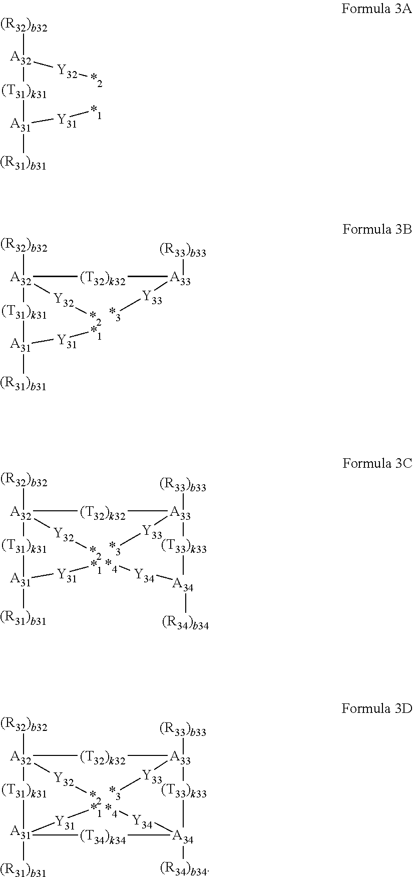

In Formulae 3A to 3D,

A₃₁ to A₃₄ may each independently be a C₃-C₆₀ carbocyclic group or a C₁-C₆₀ heterocyclic group,

T₃₁ to T₃₄ may each independently be a single bond, a double bond, *—O—*′, *—S—*′, *—C(═O)—*′, *—S(═O)—*′, *—C(R₃₅)(R₃₆)—*′, *—C(R₃₅)═C(R₃₆)—*′, *—C(R₃₅)=*′, *—Si(R₃₅)(R₃₆)—*′, *—B(R₃₅)—*′, *—N(R₃₅)—*′, or *—P(R₃₅)—*′,

k31 to k34 may each independently be 1, 2, or 3,

Y₃₁ to Y₃₄ may each independently be a single bond, *—O—*′, *—S—*′, *—C(R₃₇)(R₃₈)—*′, *—Si(R₃₇)(R₃₈)—*′, *—B(R₃₇)—*′, *—N(R₃₇)—*′, or *—P(R₃₇)—*′,

R₃₁ to R₃₈ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂), wherein R₃₁ to R₃₈ may optionally be linked together to form a substituted or unsubstituted C₅-C₆₀ carbocyclic group or a substituted or unsubstituted C₁-C₆₀ heterocyclic group,

b31 to b34 may each independently be an integer from 0 to 10, and

*₁, *₂, *₃, and *₄ each indicate a binding site to M₃₁.

In an embodiment, the organometallic compound may be represented by Formula 3-1 or 3-2:

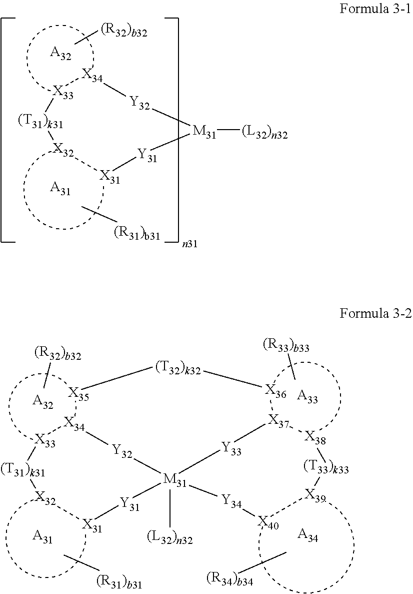

In Formulae 3-1 and 3-2,

X₃₁ to X₄₀ may each independently be selected from N and C, and

the remaining components may each independently be the same as described above in connection with Formula 3.

In Formulae 3-1 and 3-2, X₃₁ and X₃₂ may each independently be a ring member of A₃₁, and X₃₃ to X₄₀ may also be the same as X₃₁, and X₃₂ described in connection with Formulae 3-1 and 3-2. That is, X₃₃ to X₄₀ may each independently be Nor C.

In an embodiment, the organometallic compound may be selected from Group III:

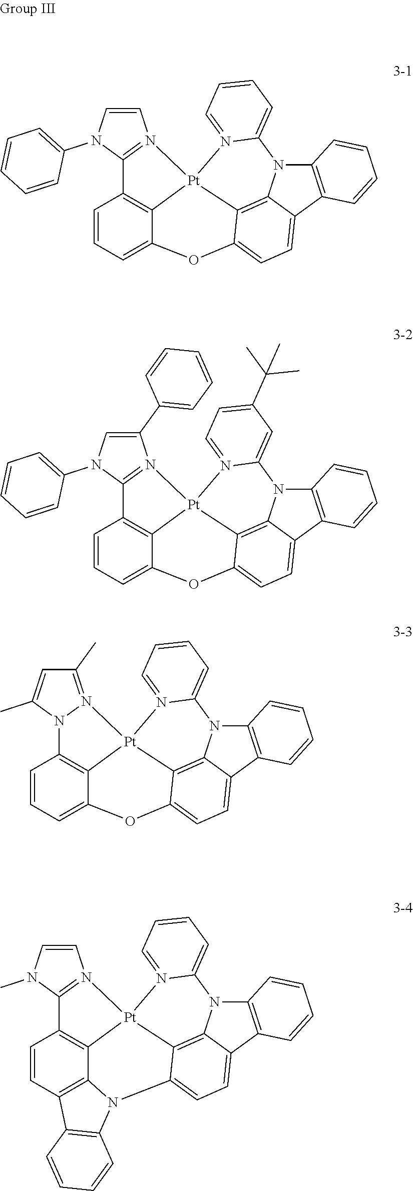

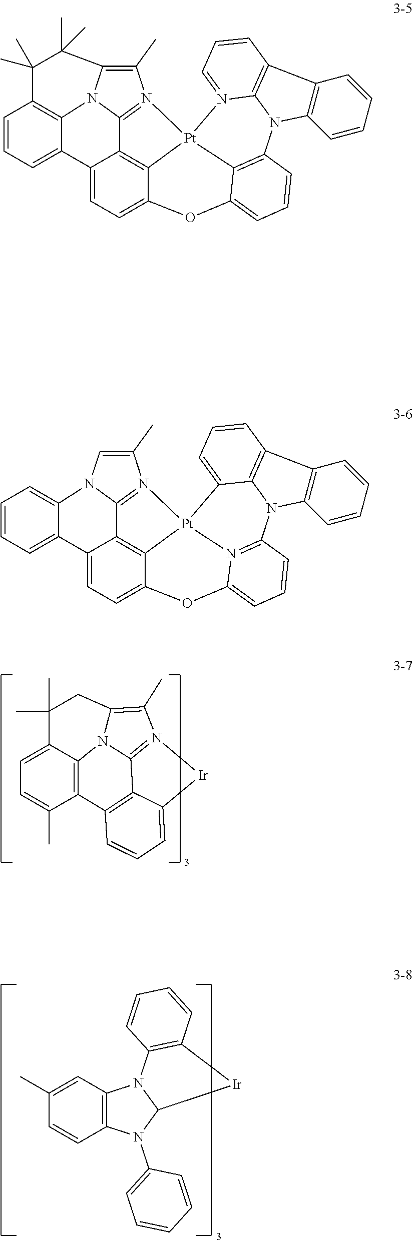

In an embodiment, the emission layer may directly contact the first layer.

In an embodiment, the emission layer may directly contact the second layer.

In an embodiment, the emission layer may directly contact each of the first layer and the second layer.

In an embodiment, the hole transport region may further include a third layer, and a difference between a highest occupied molecular orbital (HOMO) energy level of the first layer and a HOMO energy level of the third layer may be equal to or less than 0.3 eV. When the difference is within the range above, polaron-quenching, an increase in driving voltage, and deterioration at an interface with a hole transport layer due to a hole injection barrier during hole transport may be reduced, thereby minimizing or substantially minimizing reduction in the lifespan of the light-emitting device.

In an embodiment, the first layer may directly contact the third layer.

In an embodiment, the electron transport region may further include a fourth layer, and a difference between a lowest unoccupied molecular orbital (LUMO) energy level of the second layer and a LUMO energy level of the fourth layer may be equal to or less than 0.3 eV. When the difference is within the range above, polaron-quenching due to a hole injection barrier during hole transport, an increase in driving voltage, and deterioration at an interface of a hole transport layer may be reduced, thereby minimizing or substantially minimizing reduction in the lifespan of the light-emitting device.

In an embodiment, the second layer may directly contact the fourth layer.

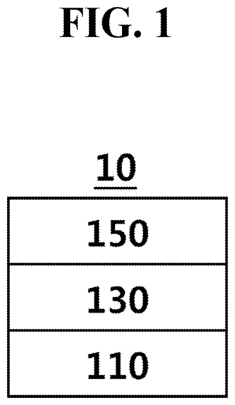

### Description of FIG. 1

FIG. 1 is a schematic cross-sectional view of a light-emitting device 10 according to an embodiment. The light-emitting device 10 includes a first electrode 110, an interlayer 130, and a second electrode 150.

Hereinafter, the structure of the light-emitting device 10 according to an embodiment of the present disclosure and a method of manufacturing the light-emitting device 10 will be described in connection with FIG. 1.

### First Electrode 110

In FIG. 1, a substrate may be additionally located under the first electrode 110 or above the second electrode 150. In an embodiment, the substrate may be a glass substrate or a plastic substrate. In one or more embodiments, the substrate may be a flexible substrate, and for example, may include plastics with suitable (e.g., excellent) heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a high work function material that can suitably (e.g., easily) inject holes may be utilized as the material for forming the first electrode 110.

The first electrode 110 may be a reflective electrode, a semi-transmissive electrode, or a transmissive electrode. In an embodiment, when the first electrode 110 is a transmissive electrode, the material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, magnesium (Mg), silver (Ag), aluminum (Al), aluminum-lithium (Al—Li), calcium (Ca), magnesium-indium (Mg—In), magnesium-silver (Mg—Ag), or any combination thereof may be utilized as the material for forming the first electrode 110.

The first electrode 110 may have a single-layered structure consisting of a single layer or a multi-layered structure including a plurality of layers. For example, the first electrode 110 may have a three-layered structure of ITO/Ag/ITO.

### Interlayer 130

The interlayer 130 is placed on the first electrode 110. The interlayer 130 may include an emission layer.

In an embodiment, the interlayer 130 may further include a hole transport region between the first electrode 110 and the emission layer and an electron transport region between the emission layer and the second electrode 150.

In one or more embodiments, the interlayer 130 may further include a metal-containing compound, such as an organometallic compound, an inorganic material, such as a quantum dot, and/or the like, in addition to various suitable organic materials.

In one or more embodiments, the interlayer 130 may include i) two or more emitting units sequentially stacked between the first electrode 110 and the second electrode 150 and ii) a charge generation layer between two adjacent emitting units selected from the two or more emitting units. When the interlayer 130 includes the two or more emitting units and the charge generation layer, the light-emitting device 10 may be a tandem light-emitting device.

### Hole Transport Region in Interlayer 130

The hole transport region may have: i) a single-layered structure consisting of a single layer consisting of a single material, ii) a single-layered structure consisting of a single layer including (e.g., consisting of) a plurality of different materials, or iii) a multi-layered structure including a plurality of layers including different materials.

The hole transport region may include a hole injection layer, a hole transport layer, an emission auxiliary layer, an electron blocking layer, or any combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein for each structure, constituting layers are sequentially stacked on the first electrode 110 in the respective stated order.

For example, the first layer may be an electron blocking layer or an emission auxiliary layer, and the third layer may be a hole transport layer. However, embodiments of the present disclosure are not limited thereto.

The hole transport region may include a compound represented by Formula 201, a compound represented by Formula 202, or any combination thereof:

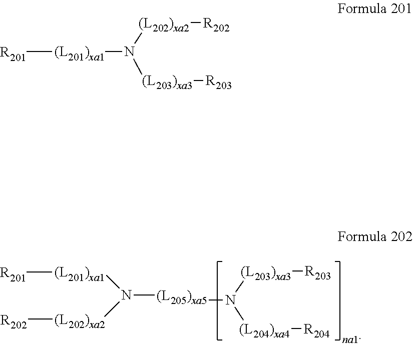

In Formulae 201 and 202,

L₂₀₁ to L₂₀₄ may each independently be a C₅-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be*—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently be an integer from 0 to 5,

xa5 may be an integer from 1 to 10,

R₂₀₁ to R₂₀₄ and Q₂₀₁ may each independently be a C₅-C₆₀ carbocyclic group unsubstituted or substituted with at least one R₁₀ₐ or a C₁-C₆₀ heterocyclic group unsubstituted or substituted with at least one R₁₀ₐ,

R₂₀₁ and R₂₀₂ may optionally be linked to each other through a single bond, a

C₁-C₅ alkylene group unsubstituted or substituted with at least one R₁₀ₐ, or a C₂-C₅ alkenylene group unsubstituted or substituted with at least one R₁₀ₐ to form a C₆-C₆₀ polycyclic group (for example, a carbazole group and/or the like) unsubstituted or substituted with at least one R₁₀ₐ (for example, see Compound HT16),

R₂₀₃ and R₂₀₄ may optionally be linked to each other through a single bond, a

C₁-C₅ alkylene group unsubstituted or substituted with at least one R₁₀ₐ, or a C₂-C₅ alkenylene group unsubstituted or substituted with at least one R₁₀ₐ to form a C₆-C₆₀ polycyclic group unsubstituted or substituted with at least one R₁₀ₐ, and

na1 may be an integer from 1 to 4.

For example, Formulae 201 and 202 may each include at least one selected from groups represented by Formulae CY201 to CY217:

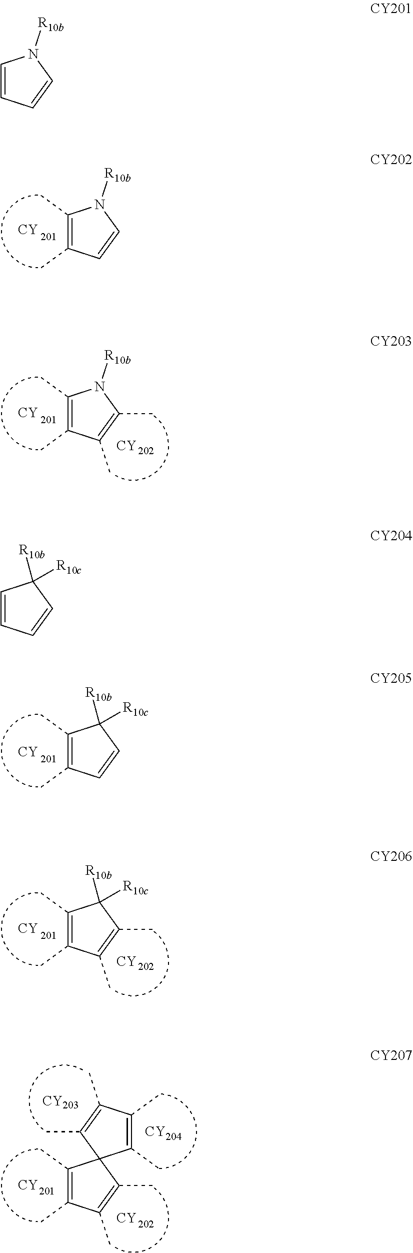

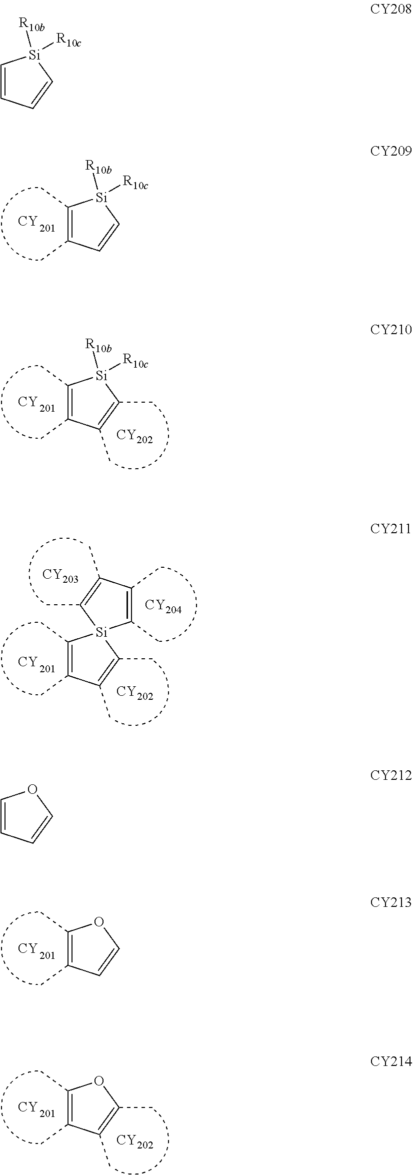

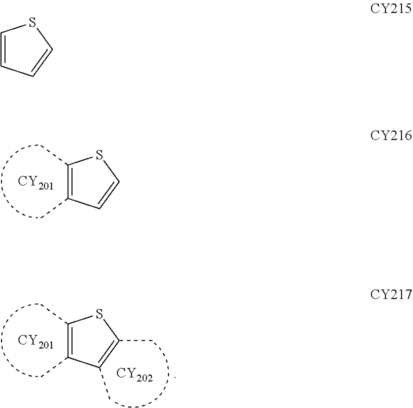

In Formulae CY201 to CY217, R_{10b} and R_{10c} may each independently be the same as described in connection with R₁₀ₐ, and ring CY₂₀₁ to ring CY₂₀₄ may each independently be a C₃-C₂₀ carbocyclic group or a C₁-C₂₀ heterocyclic group. In Formulae CY201 to CY217, at least one hydrogen may be unsubstituted or substituted with at least one R₁₀ₐ as described above.

In an embodiment, ring CY₂₀₁ to ring CY₂₀₄ in Formulae CY201 to CY217 may each independently be a benzene group, a naphthalene group, a phenanthrene group, or an anthracene group.

In one or more embodiments, Formulae 201 and 202 may each include at least one selected from groups represented by Formulae CY201 to CY203.

In one or more embodiments, Formula 201 may include at least one selected from groups represented by Formulae CY201 to CY203 and at least one selected from groups represented by Formulae CY204 to CY217.

In one or more embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In one or more embodiments, Formulae 201 and 202 may each not include groups represented by Formulae CY201 to CY203.

In one or more embodiments, Formulae 201 and 202 may each not include groups represented by Formulae CY201 to CY203, but may include at least one selected from groups represented by Formulae CY204 to CY217.

In one or more embodiments, Formulae 201 and 202 may each not include groups represented by Formulae CY201 to CY217.

For example, the hole transport region may include one of Compounds HT1 to HT44, m-MTDATA, TDATA, 2-TNATA, NPB(NPD), β-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

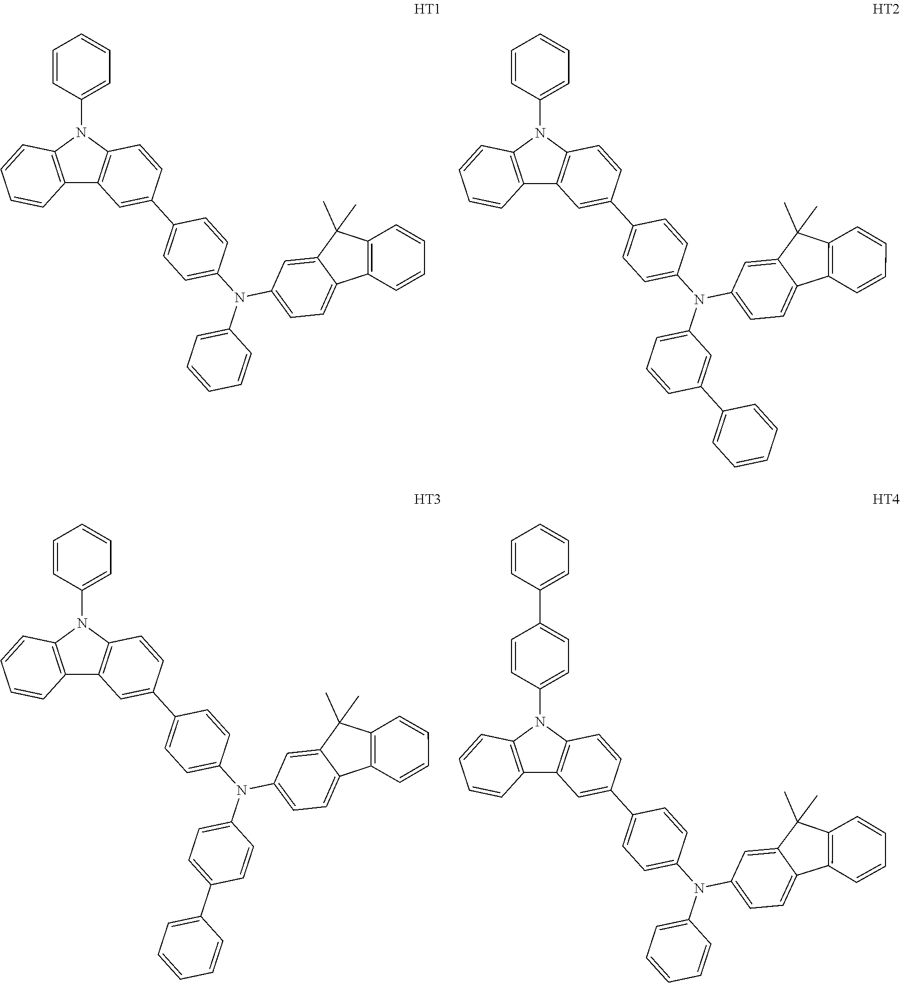

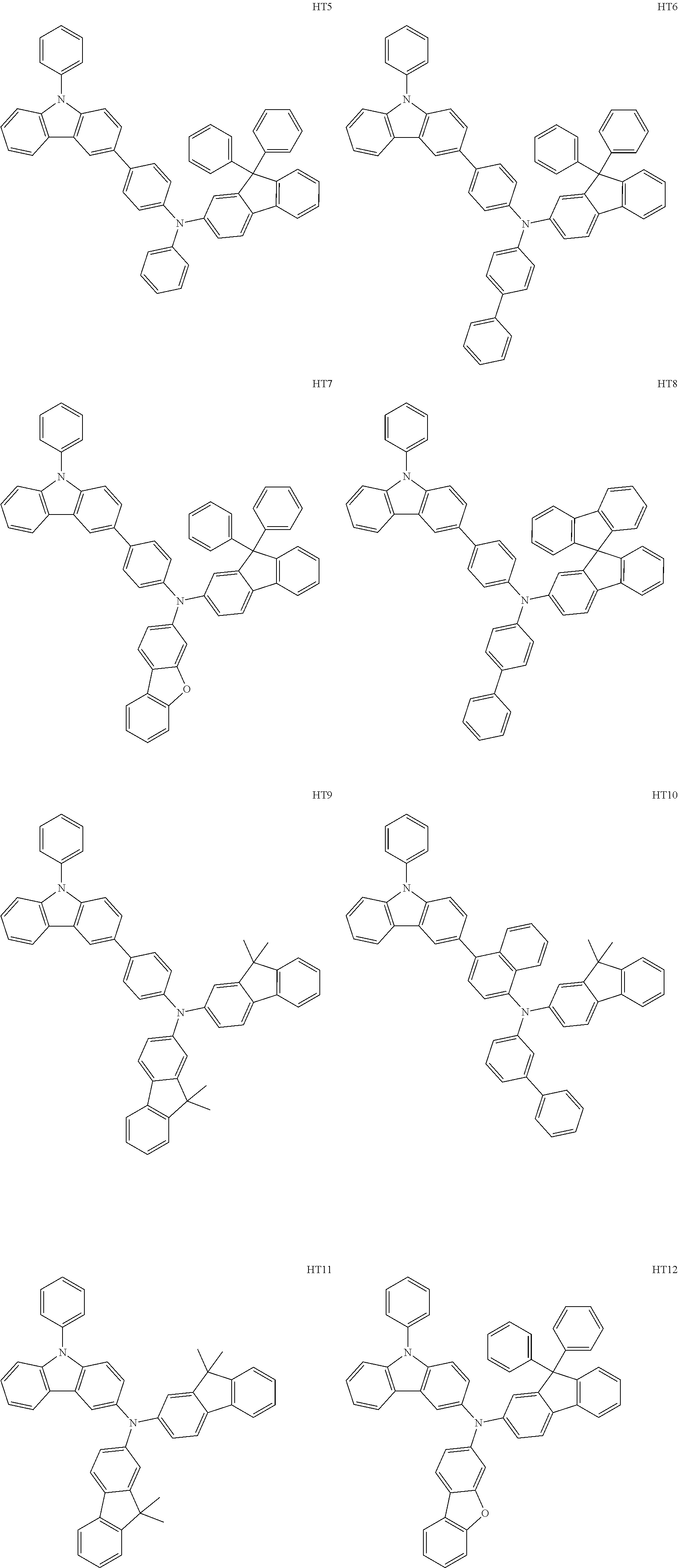

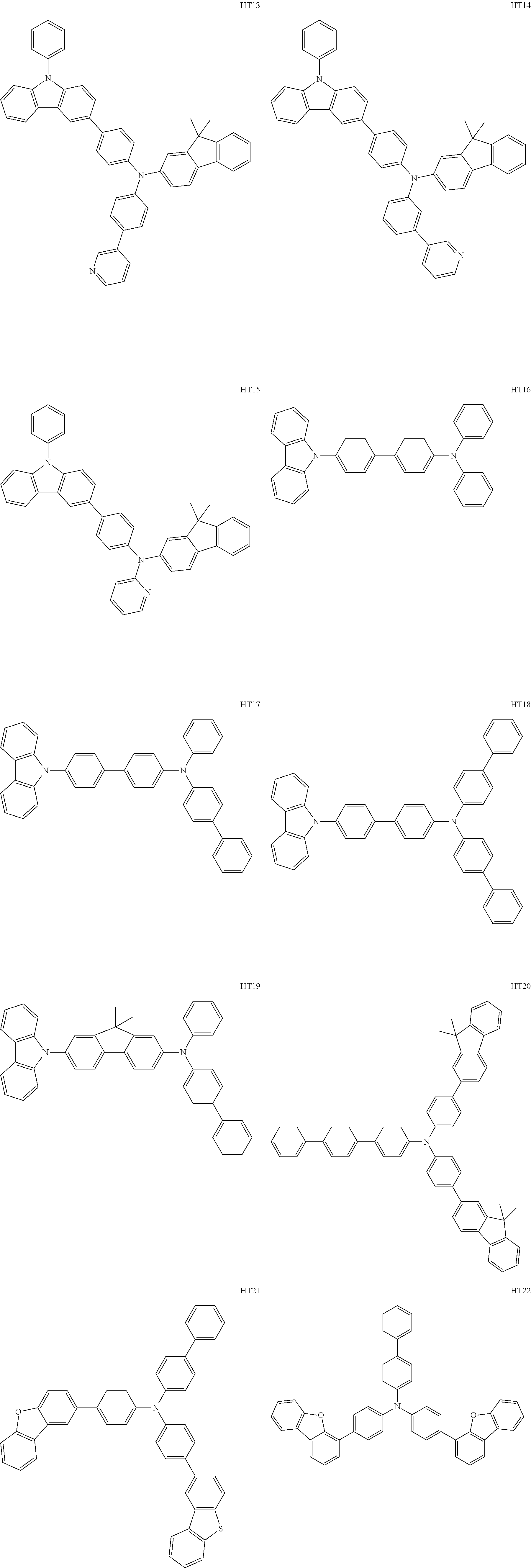

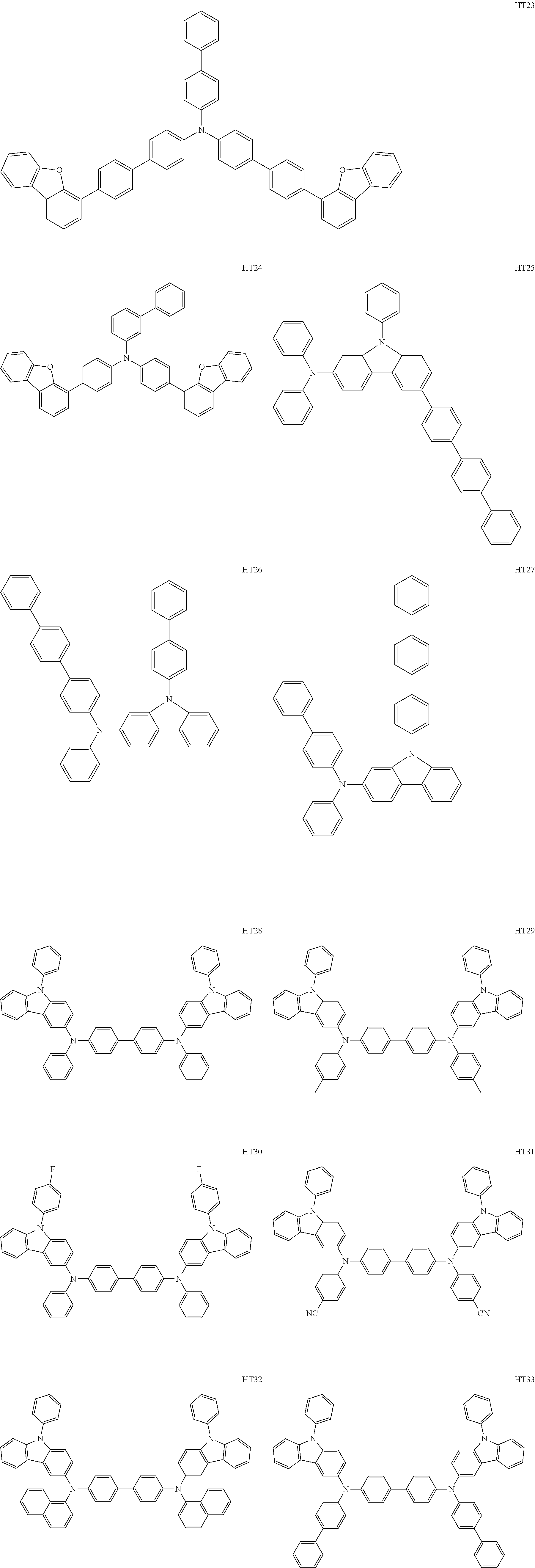

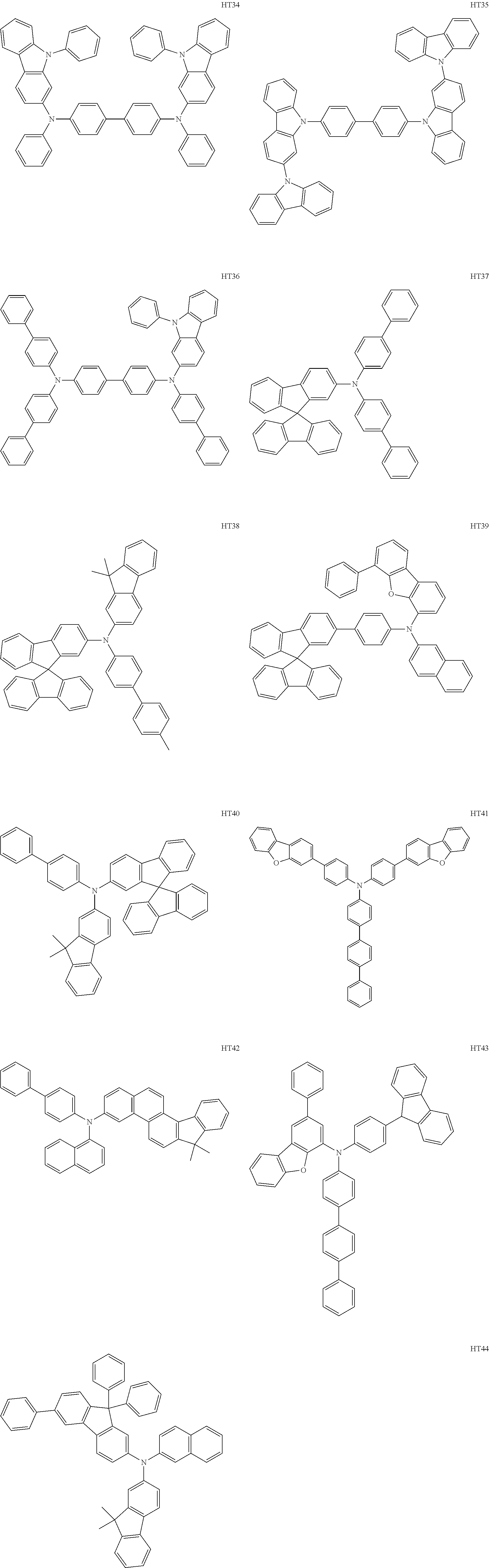

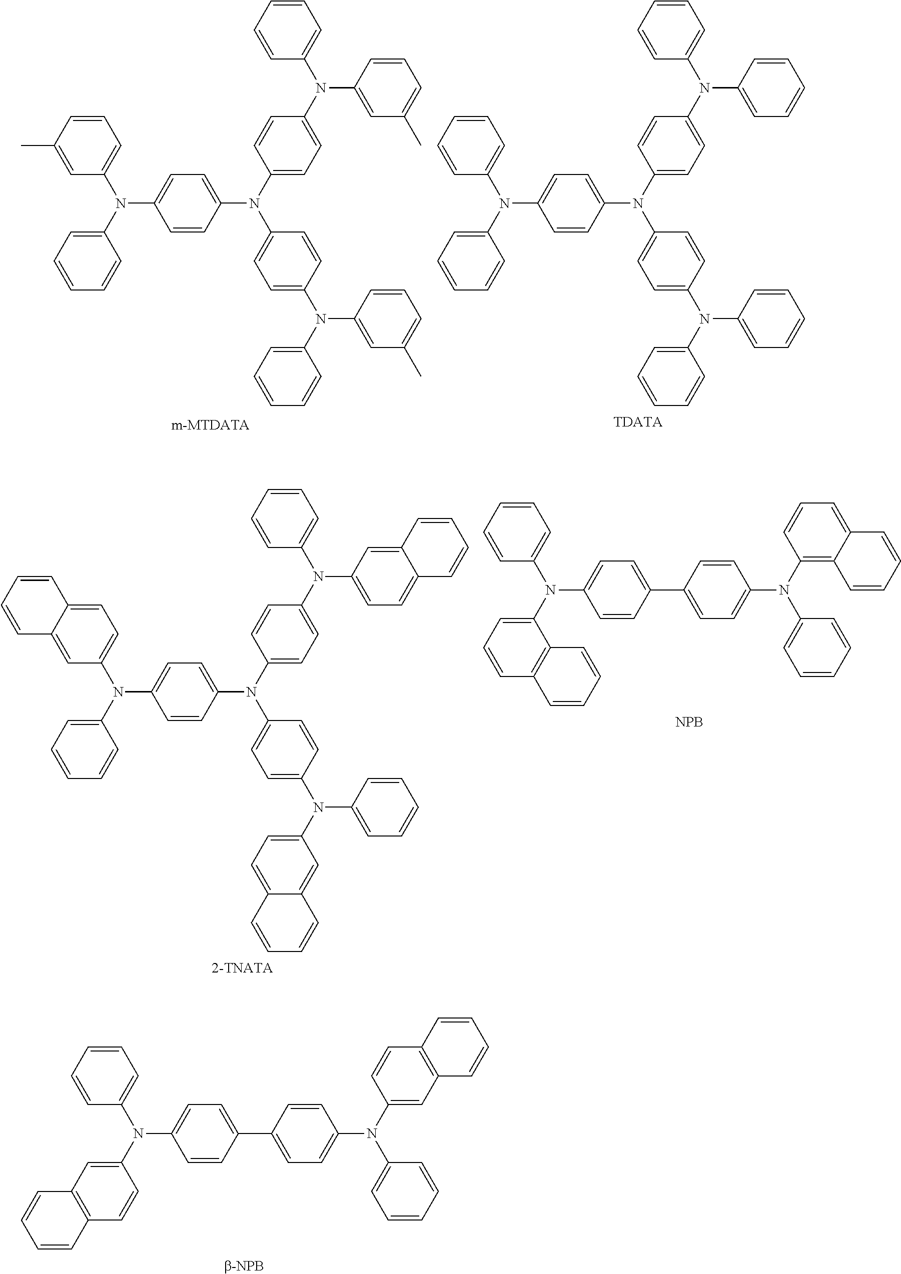

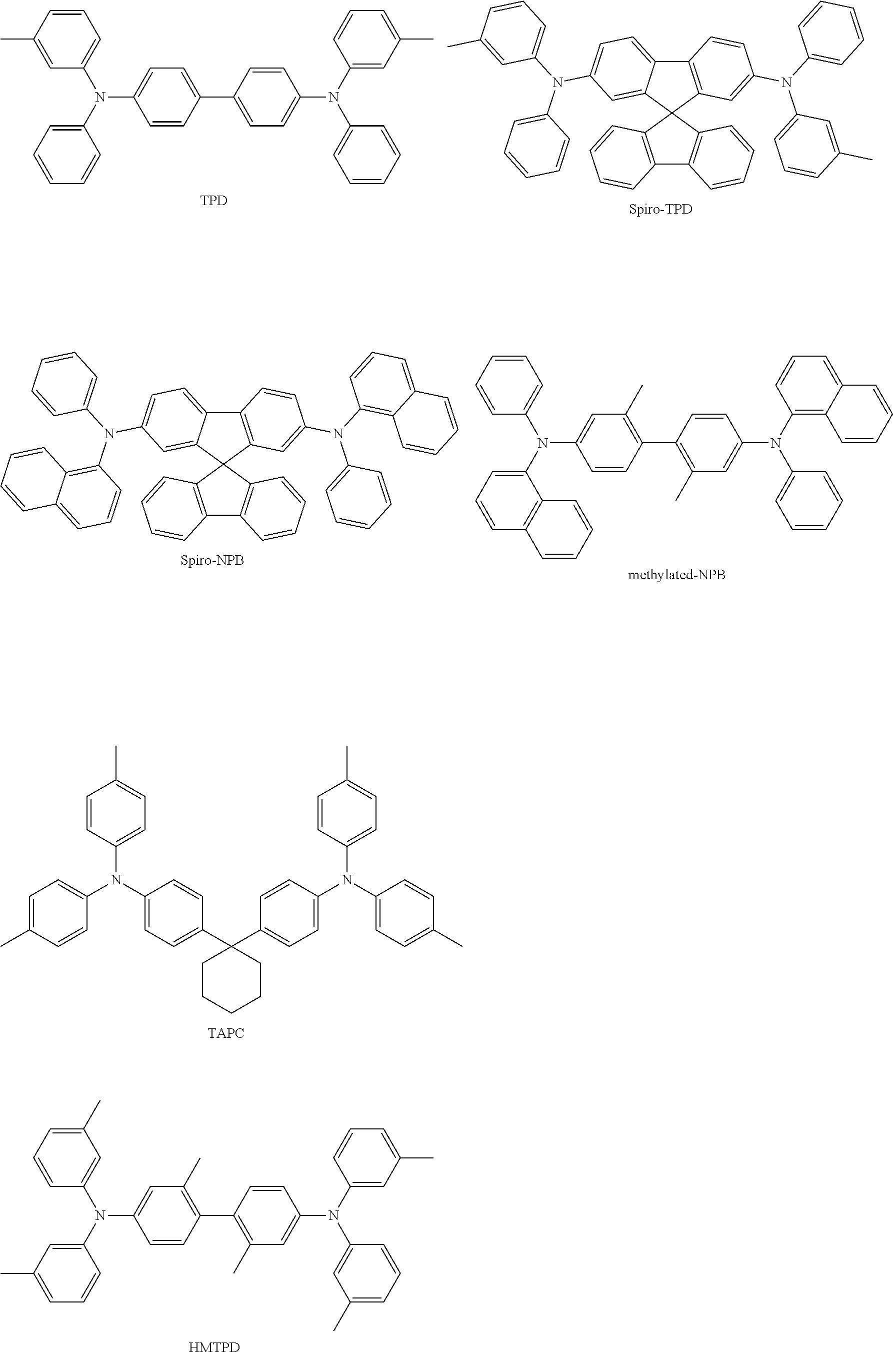

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within the ranges above, satisfactory hole transporting characteristics may be obtained without a substantial increase in driving voltage.

The emission auxiliary layer may increase light-emission efficiency by compensating for an optical resonance distance according to the wavelength of light emitted by the emission layer, and the electron blocking layer may block the flow of electrons from the electron transport region. The emission auxiliary layer and the electron blocking layer may include the materials as described above.

### P-Dopant

The hole transport region may include, in addition to these materials, a charge-generation material for the improvement of conductive properties. The charge-generation material may be uniformly or non-uniformly dispersed in the hole transport region (for example, in the form of a single layer including (e.g., consisting of) a charge-generation material).

The charge-generation material may be, for example, a p-dopant.

For example, a LUMO energy level of the p-dopant may be equal to or less than −3.5 eV.

In an embodiment, the p-dopant may include a quinone derivative, a cyano group-containing compound, a compound including Elements EL1 and EL2 (to be described in more detail below), or any combination thereof.

Non-limiting examples of the quinone derivative are TCNQ, F4-TCNQ, and/or the like.

Non-limiting examples of the cyano group-containing compound are HAT-CN, a compound represented by Formula 221, and/or the like.

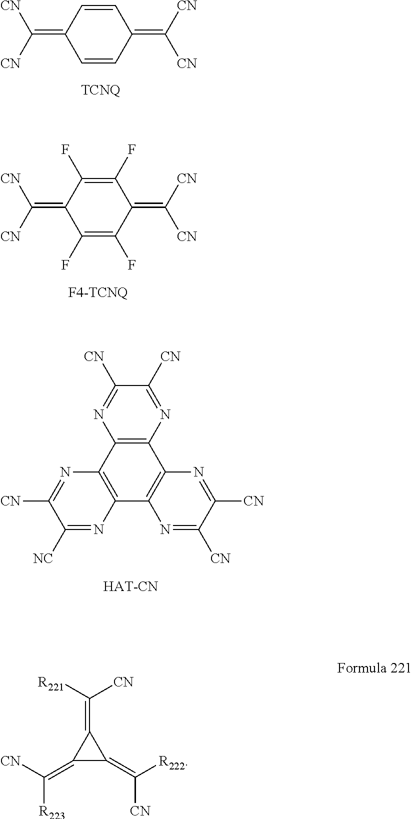

In Formula 221,

R₂₂₁ to R₂₂₃ may each independently be a C₅-C₆₀ carbocyclic group unsubstituted or substituted with at least one R₁₀ₐ or a C₁-C₆₀ heterocyclic group unsubstituted or substituted with at least one R₁₀ₐ, and

at least one of R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

Regarding the compound including Elements EL1 and EL2, Element EU may be a metal, a metalloid, or a combination thereof, and Element EL2 may be a non-metal, a metalloid, or a combination thereof.

Non-limiting examples of the metal are: an alkali metal (for example, lithium (Li), sodium (Na), potassium (K), rubidium (Rb), cesium (Cs), etc.); an alkali earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc); a post transition metal (for example, zinc (Zn), indium (In), tin (Sn), etc.); a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.); and/or the like.

Non-limiting examples of the metalloid are silicon (Si), antimony (Sb), tellurium (Te), and/or the like.

Non-limiting examples of the non-metal are oxygen (O), halogen (for example, F, Cl, Br, I, etc.), and/or the like.

Non-limiting examples of the compound including Elements EL1 and EL2 are a metal oxide, a metal halide (for example, metal fluoride, metal chloride, metal bromide, metal iodide, etc.), a metalloid halide (for example, metalloid fluoride, metalloid chloride, metalloid bromide, metalloid iodide, etc.), a metal telluride, or any combination thereof.

Non-limiting examples of the metal oxide are tungsten oxide (for example, WO, W₂O₃, WO₂, WO₃, W₂O₅, etc.), vanadium oxide (for example, VO, V₂O₃, VO₂, V₂O₅, etc.), molybdenum oxide (MoO, Mo₂O₃, MoO₂, MoO₃, Mo₂O₅, etc.), rhenium oxide (for example, ReO₃, etc.), and/or the like.

Non-limiting examples of the metal halide are alkali metal halide, alkali earth metal halide, transition metal halide, post transition metal halide, lanthanide metal halide, and/or the like.

Non-limiting examples of the alkali metal halide are LiF, NaF, KF, RbF, CsF, LiCl, NaCl, KCl, RbCl, CsCl, LiBr, NaBr, KBr, RbBr, CsBr, LiI, NaI, KI, RbI, CsI, and/or the like.

Non-limiting examples of the alkali earth metal halide are BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, BaI₂, and/or the like.

Non-limiting examples of the transition metal halide are titanium halide (for example, TiF₄, TiCl₄, TiBr₄, Tii₄, etc.), zirconium halide (for example, ZrF₄, ZrCl₄, ZrBr₄, Zri₄, etc.), hafnium halide (for example, HfF₄, HfCl₄, HfBr₄, Hfi₄, etc.), vanadium halide (for example, VF₃, VCl₃, VBr₃, VI₃, etc.), niobium halide (for example, NbF₃, NbC₁₃, NbBr₃, Nbi₃, etc.), tantalum halide (for example, TaF₃, TaCl₃, TaBr₃, Tai₃, etc.), chromium halide (for example, CrF₃, CrCl₃, CrBr₃, Cris, etc.), molybdenum halide (for example, MoF₃, MoCl₃, MoBr₃, MoI₃, etc.), tungsten halide (for example, WF₃, WCl₃, WBr₃, WI₃, etc.), manganese halide (for example, MnF₂, MnCl₂, MnBr₂, MnI₂, etc.), technetium halide (for example, TcF₂, TcCl₂, TcBr₂, TcI₂, etc.), rhenium halide (for example, ReF₂, ReCl₂, ReBr₂, ReI₂, etc.), iron halide (for example, FeF₂, FeCl₂, FeBr₂, FeI₂, etc.), ruthenium halide (for example, RuF₂, RuCl₂, RuBr₂, RuI₂, etc.), osmium halide (for example, OsF₂, OsCl₂, OsBr₂, OsI₂, etc.), cobalt halide (for example, CoF₂, CoCl₂, CoBr₂, CoI₂, etc.), rhodium halide (for example, RhF₂, RhCl₂, RhBr₂, RhI₂, etc.), iridium halide (for example, IrF₂, IrCl₂, IrBr₂, Ir₁₂, etc.), nickel halide (for example, NiF₂, NiCl₂, NiBr₂, NiI₂, etc.), palladium halide (for example, PdF₂, PdC₁₂, PdBr₂, PdI₂, etc.), platinum halide (for example, PtF₂, PtC₁₂, PtBr₂, PtI₂, etc.), copper halide (for example, CuF, CuCl, CuBr, CuI, etc.), silver halide (for example, AgF, AgCl, AgBr, AgI, etc.), gold halide (for example, AuF, AuCl, AuBr, AuI, etc.), and/or the like.

Non-limiting examples of the post transition metal halide are zinc halide (for example, ZnF₂, ZnCl₂, ZnBr₂, ZnI₂, etc.), indium halide (for example, InI₃, etc.), tin halide (for example, SnI₂, etc.), and/or the like.

Non-limiting examples of the lanthanide metal halide are YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and/or the like.

Non-limiting examples of the metalloid halide are antimony halide (for example, SbCl₅, etc.), and/or the like.

Non-limiting examples of the metal telluride are alkali metal telluride (for example, Li₂Te, Na₂Te, K₂Te, Rb₂Te, Cs₂Te, etc.), alkali earth metal telluride (for example, BeTe, MgTe, CaTe, SrTe, BaTe, etc.), transition metal telluride (for example,

TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post transition metal telluride (for example, ZnTe, etc.), lanthanide metal telluride (for example, LaTe, CeTe, PrTe, NdTe, PmTe, EuTe, GdTe, TbTe, DyTe, HoTe, ErTe, TmTe, YbTe, LuTe, etc.), and/or the like.

### Emission Layer in Interlayer 130

When the light-emitting device 10 is a full-color light-emitting device, the emission layer may be patterned into a red emission layer, a green emission layer, and/or a blue emission layer, according to a sub-pixel. In an embodiment, the emission layer may have a stacked structure of two or more layers of a red emission layer, a green emission layer, and a blue emission layer, in which the two or more layers contact each other or are separated from each other. In one or more embodiments, the emission layer may include two or more materials of a red light-emitting material, a green light-emitting material, and a blue light-emitting material, in which the two or more materials are mixed with each other in a single layer to emit white light.

In an embodiment, the emission layer may further include an additional host other than the first host and the second host.

In one or more embodiments, the emission layer may further include a quantum dot.

In one or more embodiments, the emission layer may further include a delayed fluorescence material.

A thickness of the emission layer may be in a range of about 100 Å to about 1,000 Å, for example, about 200 Å to about 600 Å. When the thickness of the emission layer is within the ranges above, suitable (e.g., excellent) light-emission characteristics may be obtained without a substantial increase in driving voltage.

### Host

The host may include a compound represented by Formula 301: Formula 301

[Ar₃₀₁]_{xb11}-[(L₃₀₁)_{xb1}-R₃₀₁]_{xb21}

wherein, in Formula 301,

Ar₃₀₁ and L₃₀₁ may each independently be a C₅-C₆₀ carbocyclic group unsubstituted or substituted with at least one R₁₀ₐ or a C₁-C₆₀ heterocyclic group unsubstituted or substituted with at least one R₁₀ₐ,

xb11 may be 1, 2, or 3,

xb1 may be an integer from 0 to 5,

R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

xb21 may be an integer from 0 to 5, and

Q₃₀₁ to Q₃₀₃ may each independently be the same as described in connection with Q₁.

In an embodiment, when xb11 in Formula 301 is 2 or more, two or more of Ar₃₀₁(s) may be linked to each other via a single bond.

In one or more embodiments, the host may include a compound represented by Formula 301-1, a compound represented by Formula 301-2, or any combination thereof:

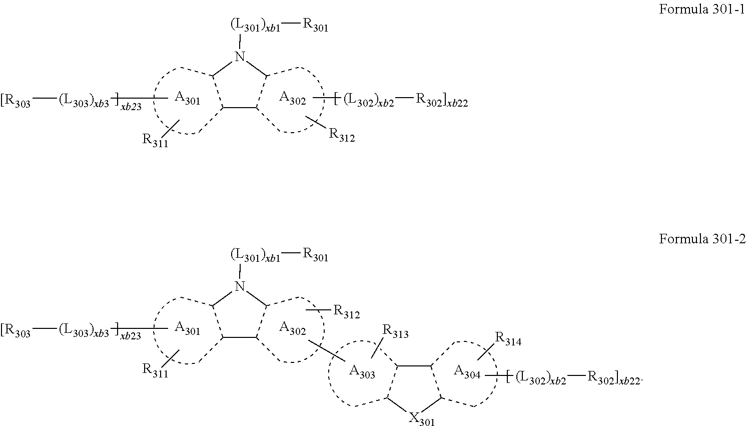

In Formulae 301-1 and 301-2,

ring A₃₀₁ to ring A₃₀₄ may each independently be a C₅-C₆₀ carbocyclic group unsubstituted or substituted with at least one R₁₀ₐ or a C₁-C₆₀ heterocyclic group unsubstituted or substituted with at least one R₁₀ₐ,

X₃₀₁ may be O, S, N-[(L₃₀₄)_{xb4}-R₃₀₄], C(R₃₀₄)(R₃₀₅), or Si(R₃₀₄)(R₃₀₅),

xb22 and xb23 may each independently be 0, 1, or 2,

L₃₀₁, xb1, and R₃₀₁ may each the same as described above,

L₃₀₂ to L₃₀₄ may each independently be the same as described in connection with L₃₀₁,

xb2 to xb4 may each independently be the same as described in connection with xb1, and

R₃₀₂ to R₃₀₅ and R₃₁₁ to R₃₁₄ may each independently be the same as described in connection with R₃₀₁.

In one or more embodiments, the host may include an alkaline earth metal complex. In one or more embodiments, the host may be a Be complex (for example, Compound H55), a Mg complex, a Zn complex, or any combination thereof.

In one or more embodiments, the host may include one of Compounds H1 to H124, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof:

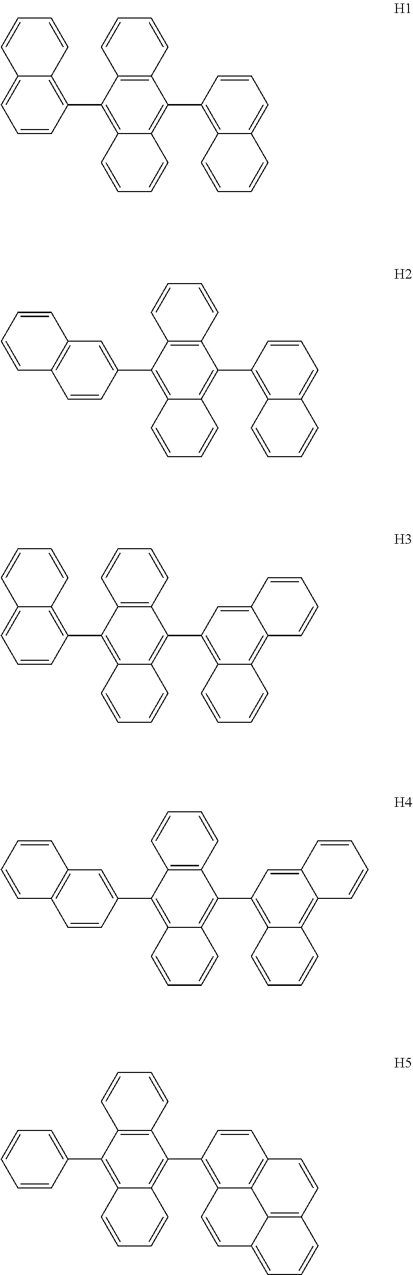

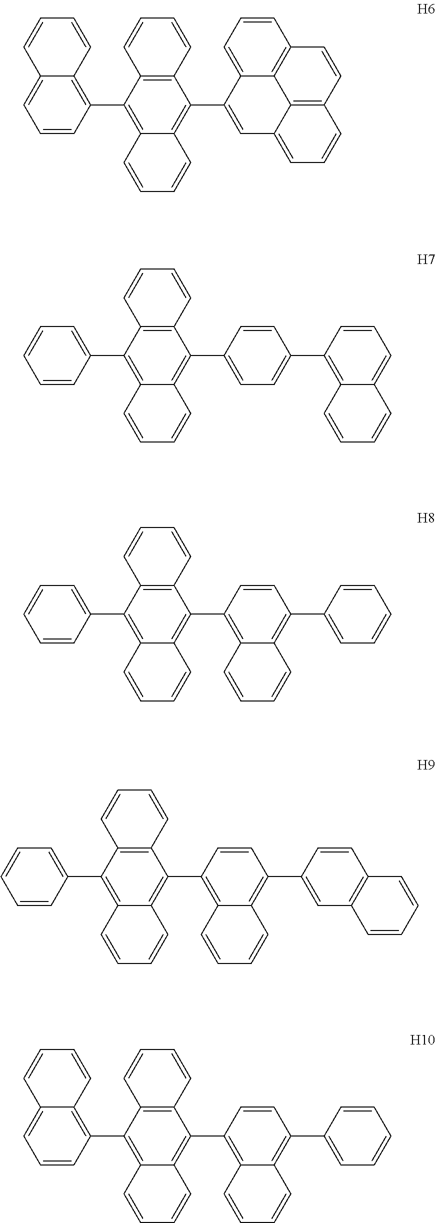

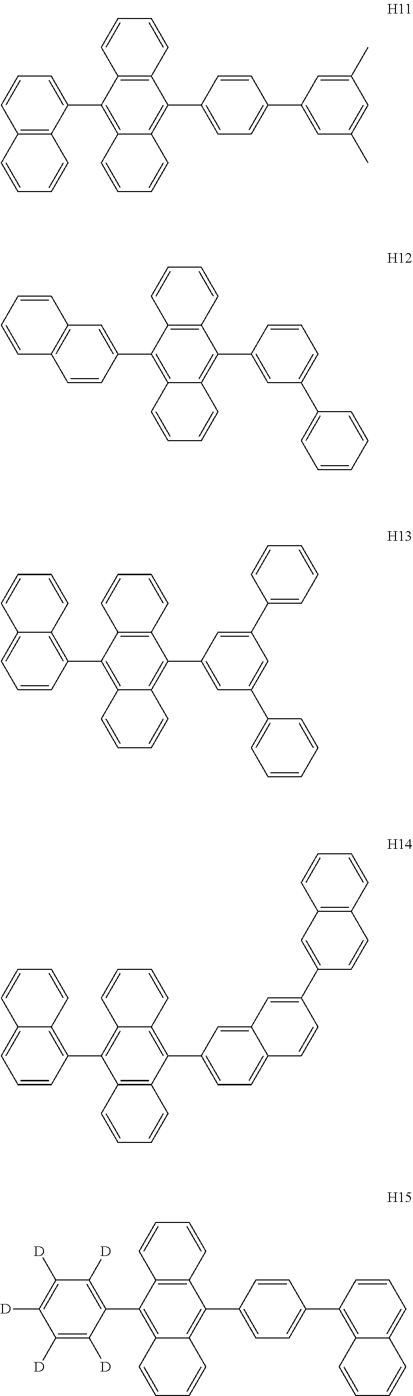

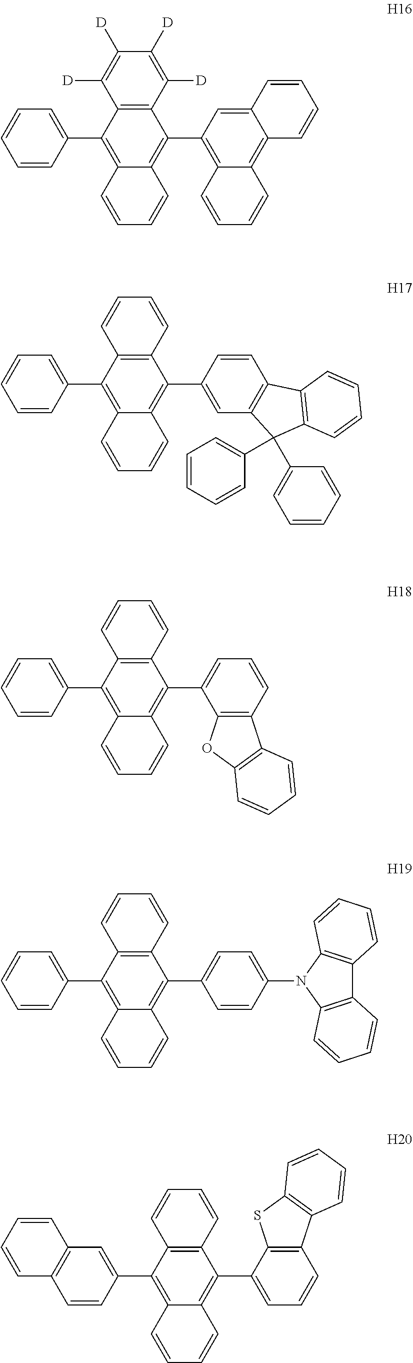

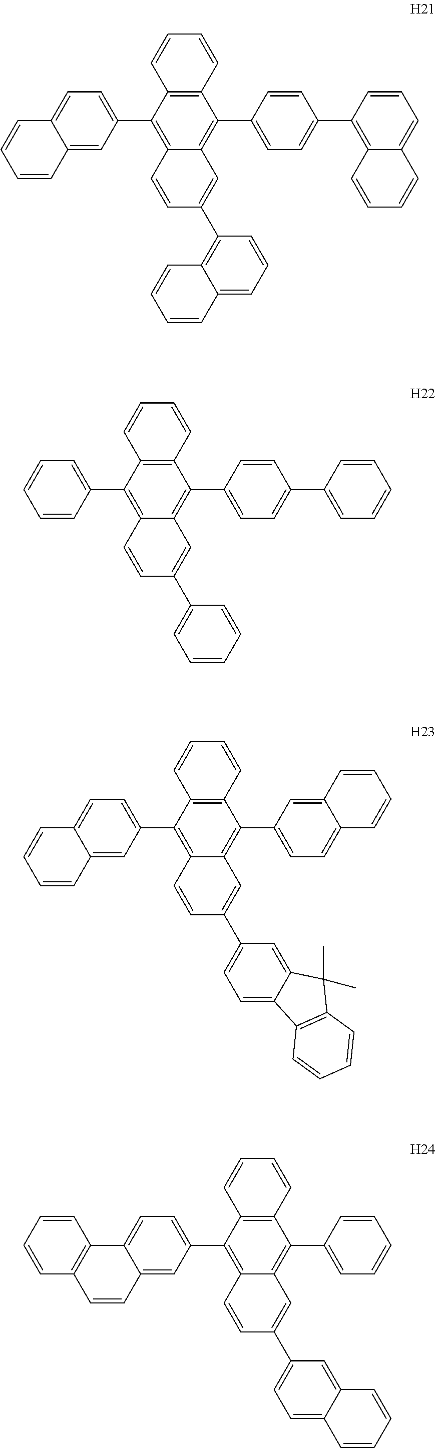

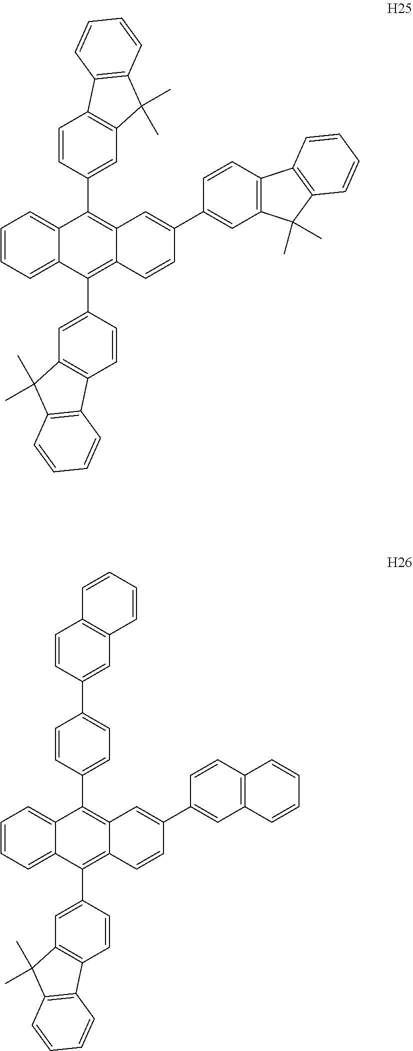

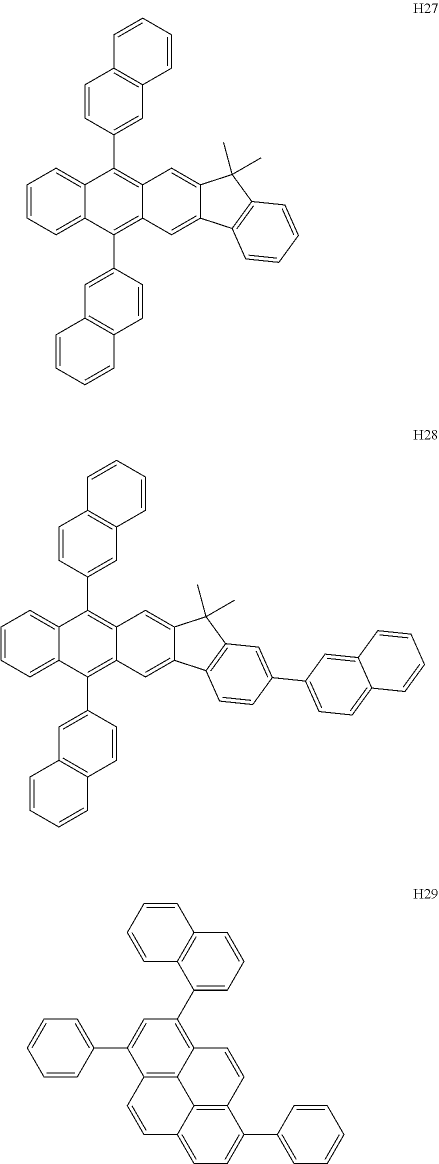

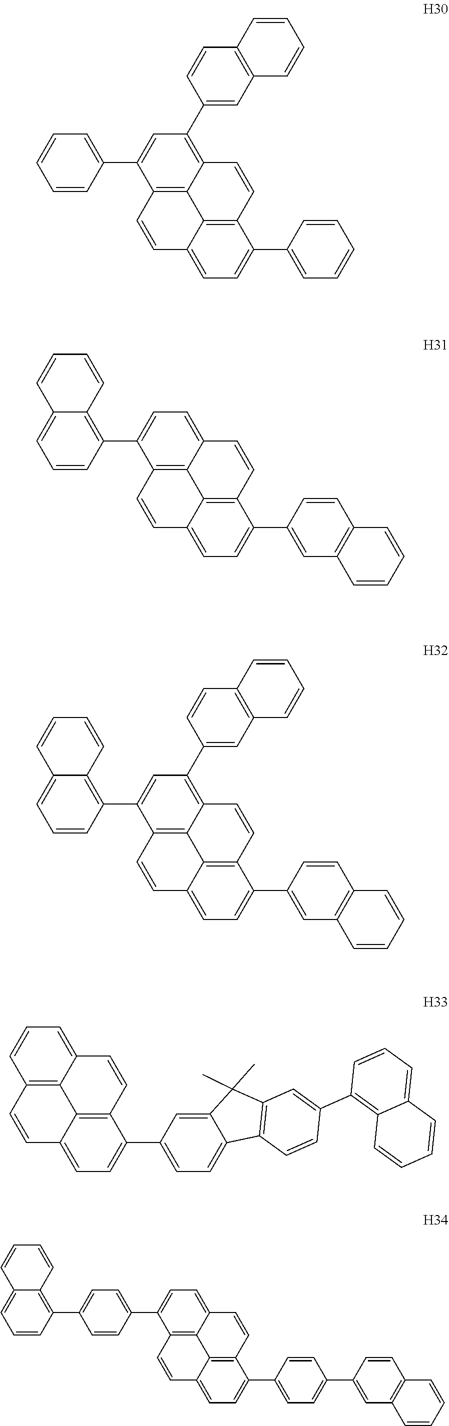

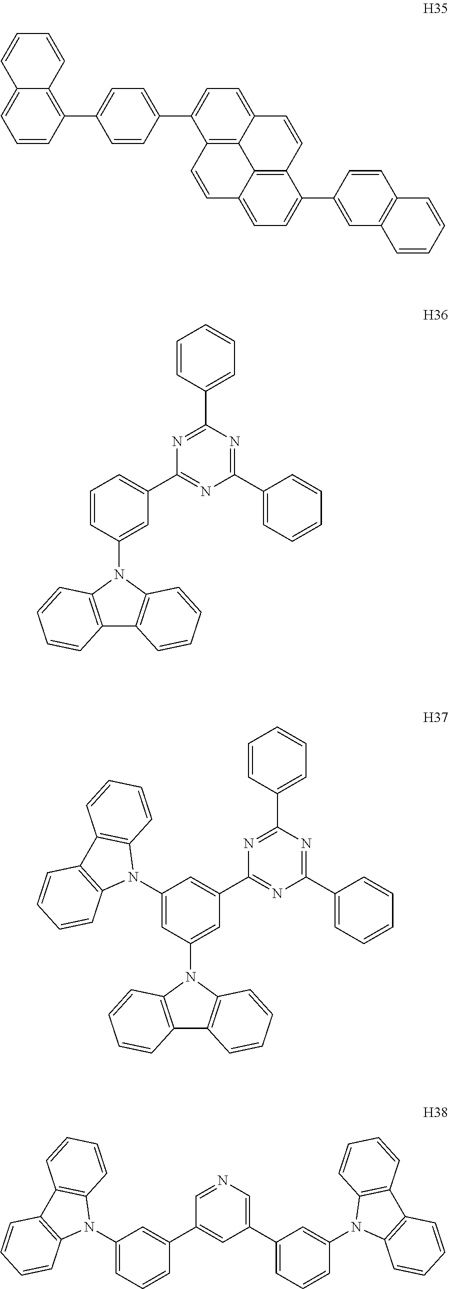

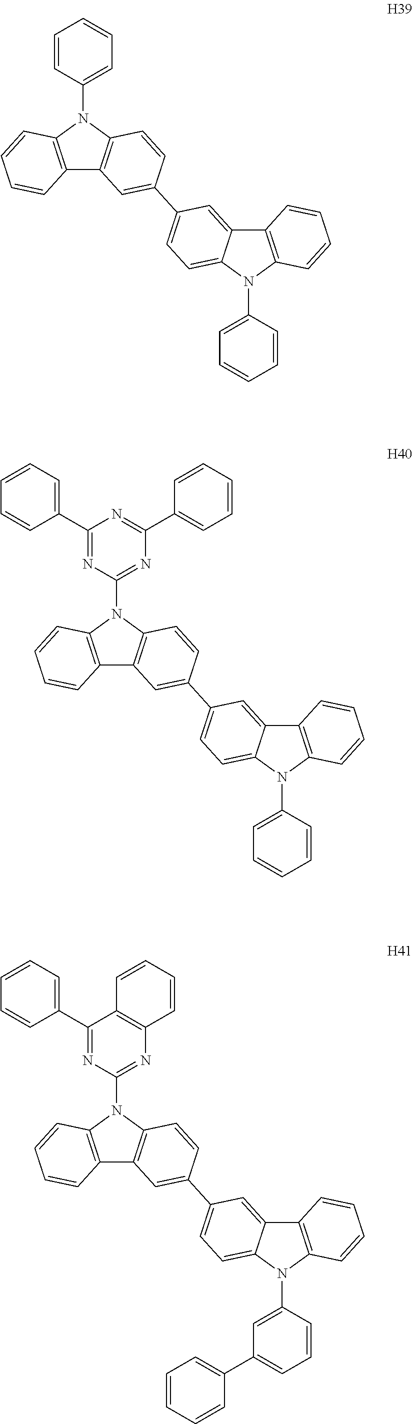 [SMILES: c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1]

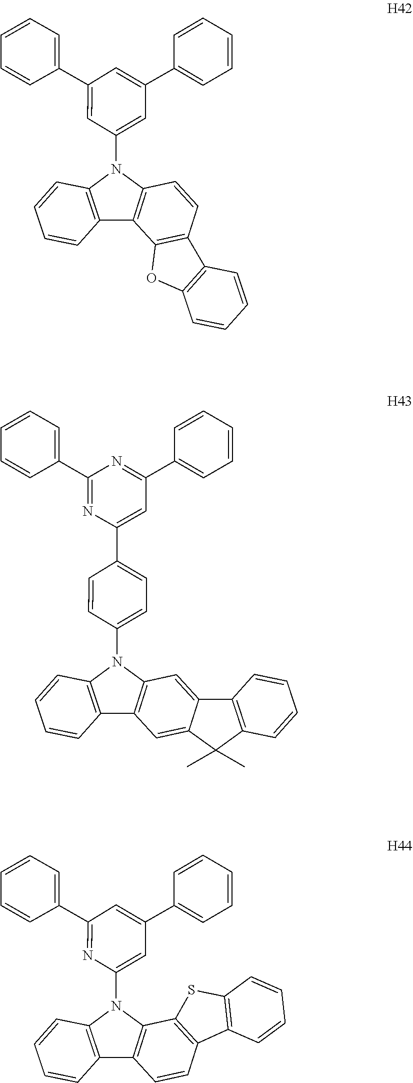 [SMILES: c1ccc(-c2cc(-c3ccccc3)cc(-n3c4ccccc4c4c5oc6ccccc6c5ccc43)c2)cc1 ; CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1]

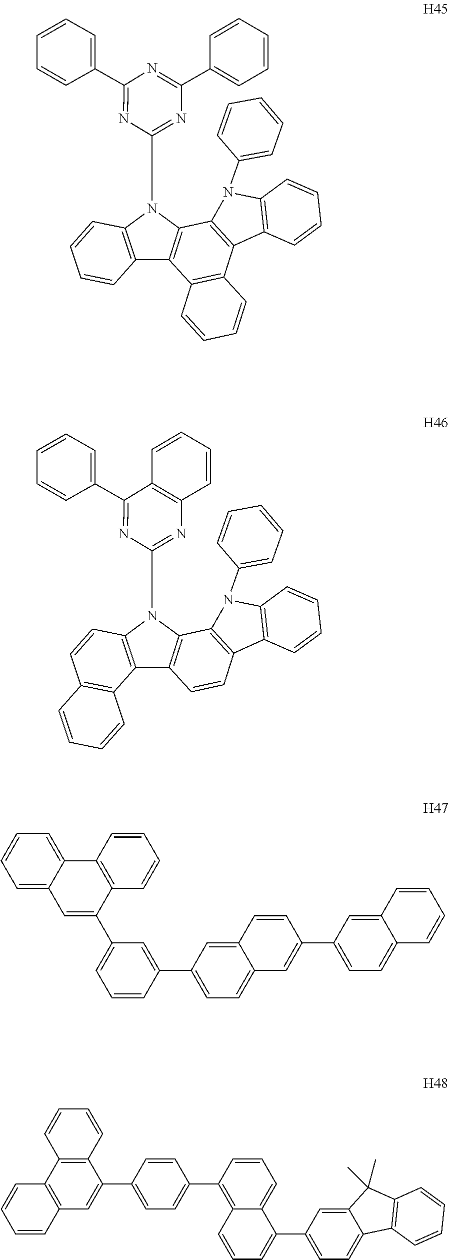

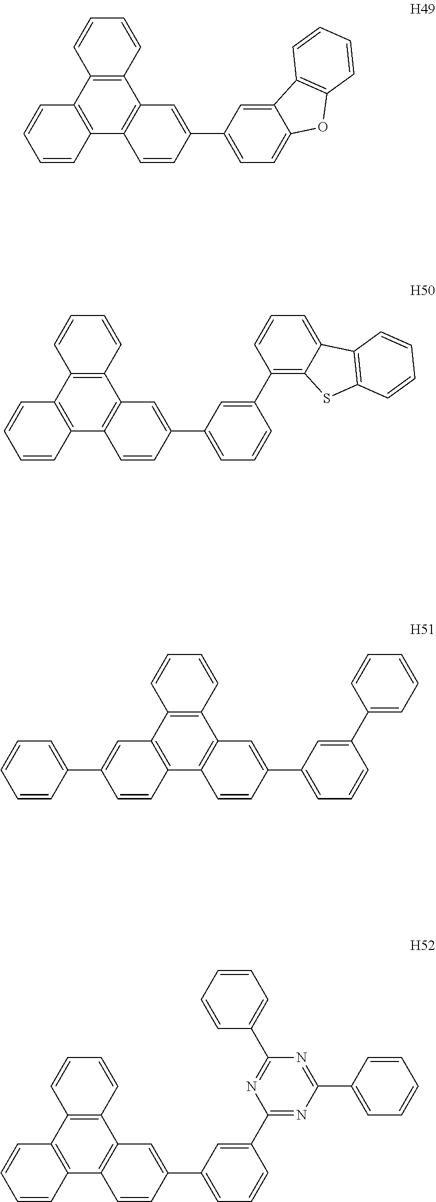

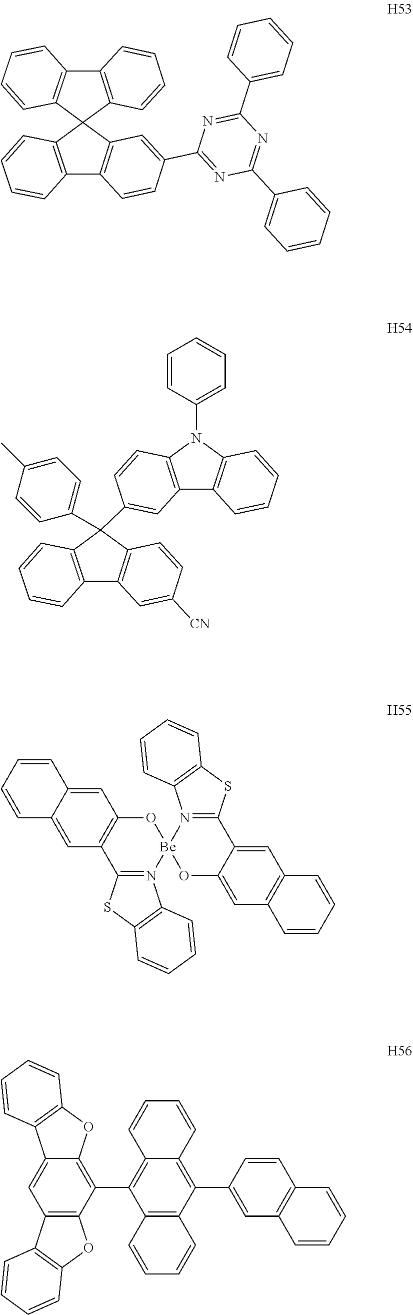

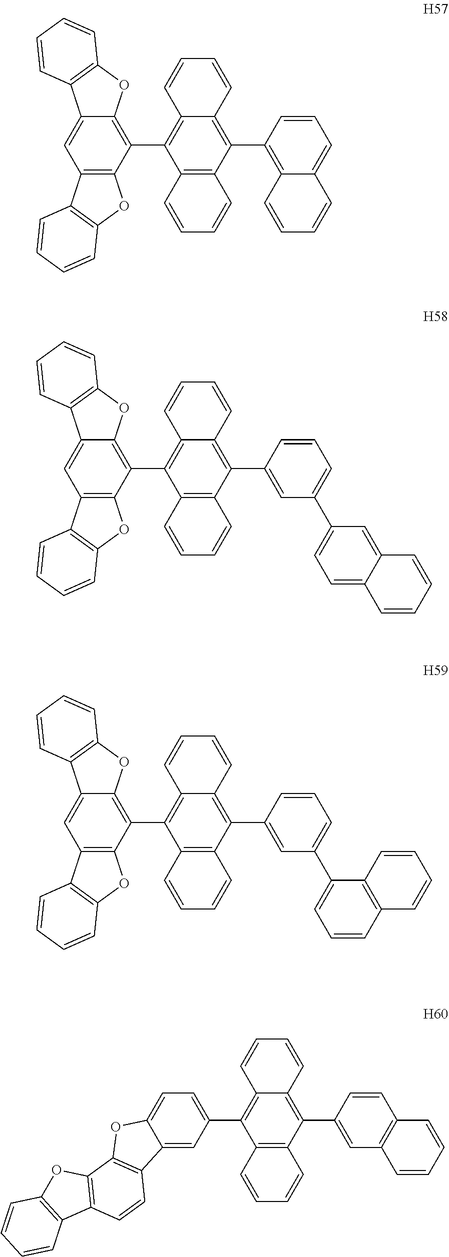 [SMILES: c1ccc2c(-c3c4ccccc4c(-c4c5oc6ccccc6c5cc5c4oc4ccccc45)c4ccccc34)cccc2c1 ; c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1]

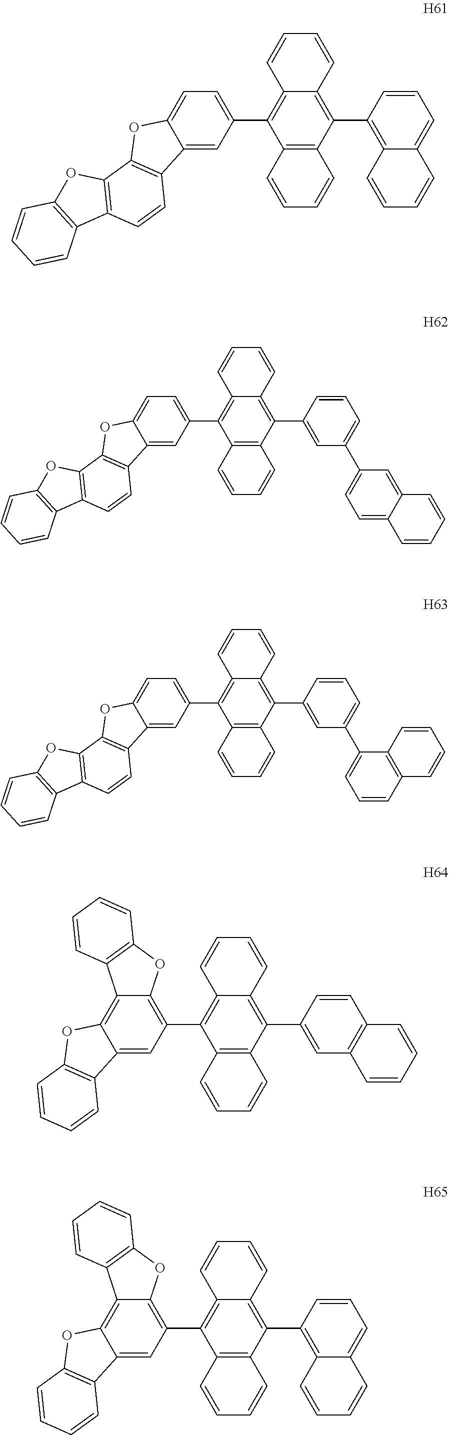 [SMILES: c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1 ; c1ccc2c(-c3c4ccccc4c(-c4cc5c6ccccc6oc5c5c4oc4ccccc45)c4ccccc34)cccc2c1]

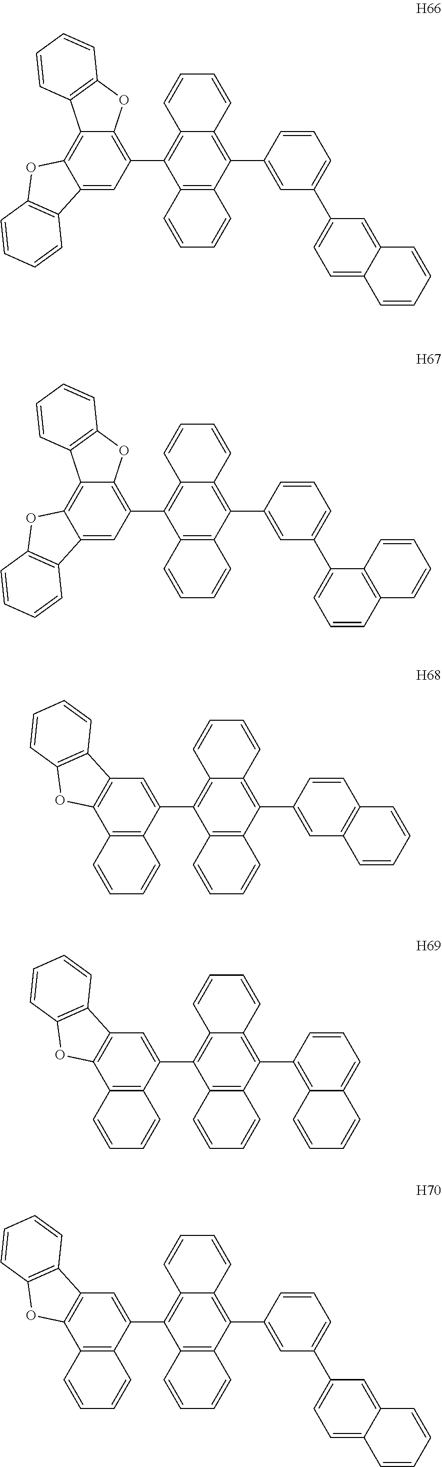 [SMILES: c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1 ; c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1]

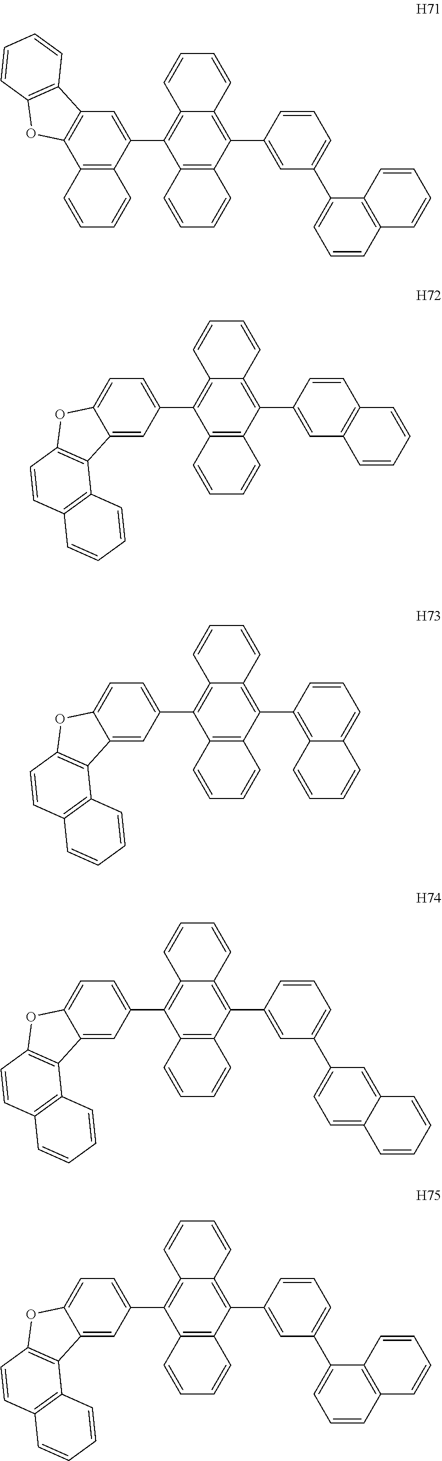 [SMILES: c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1 ; c1ccc2c(-c3c4ccccc4c(-c4ccc5oc6ccc7ccccc7c6c5c4)c4ccccc34)cccc2c1 ; c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1]

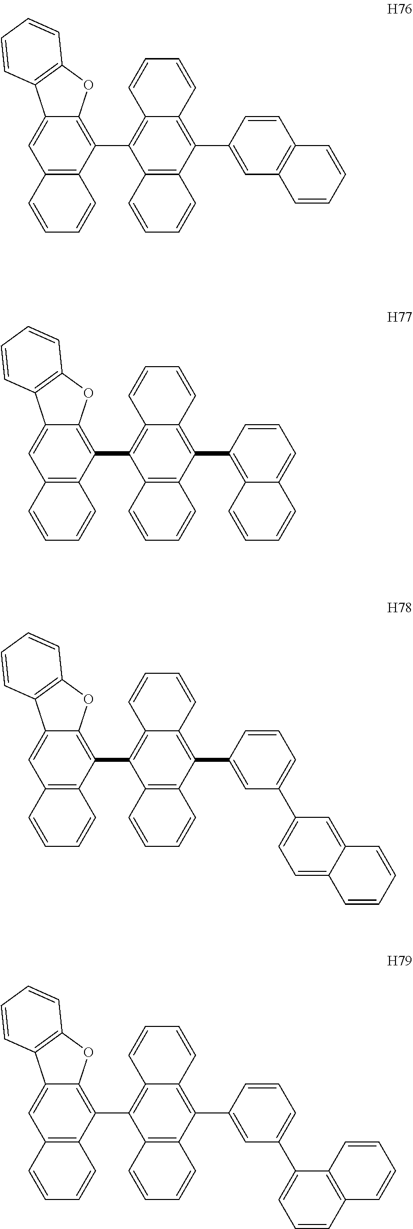

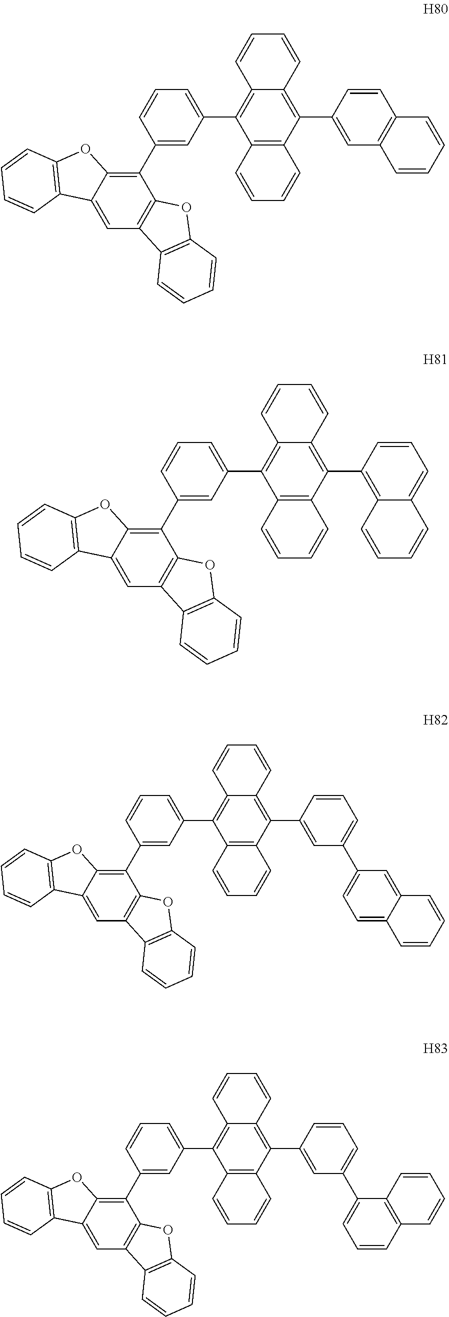 [SMILES: c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1 ; c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1]

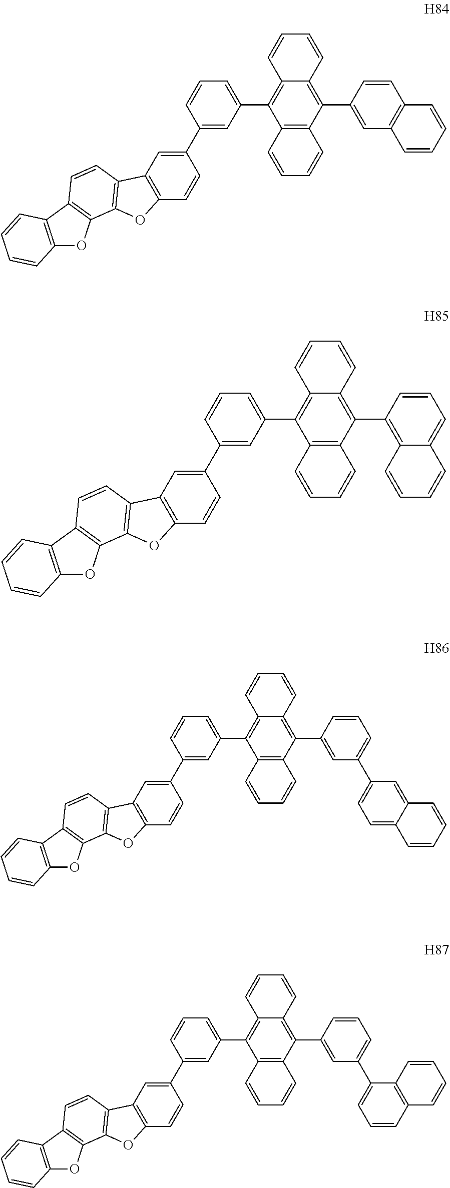 [SMILES: c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1]

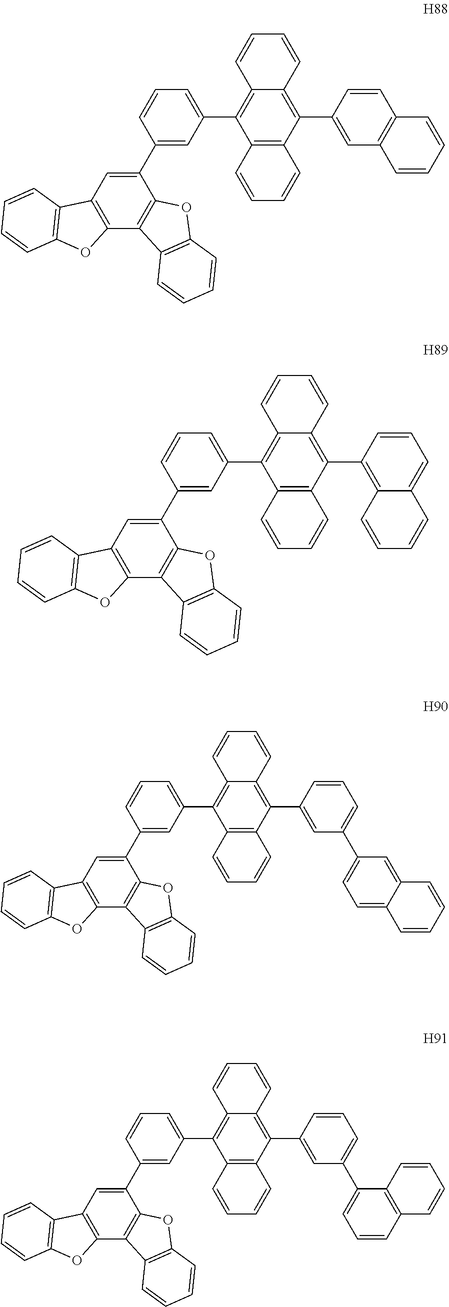 [SMILES: c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1]

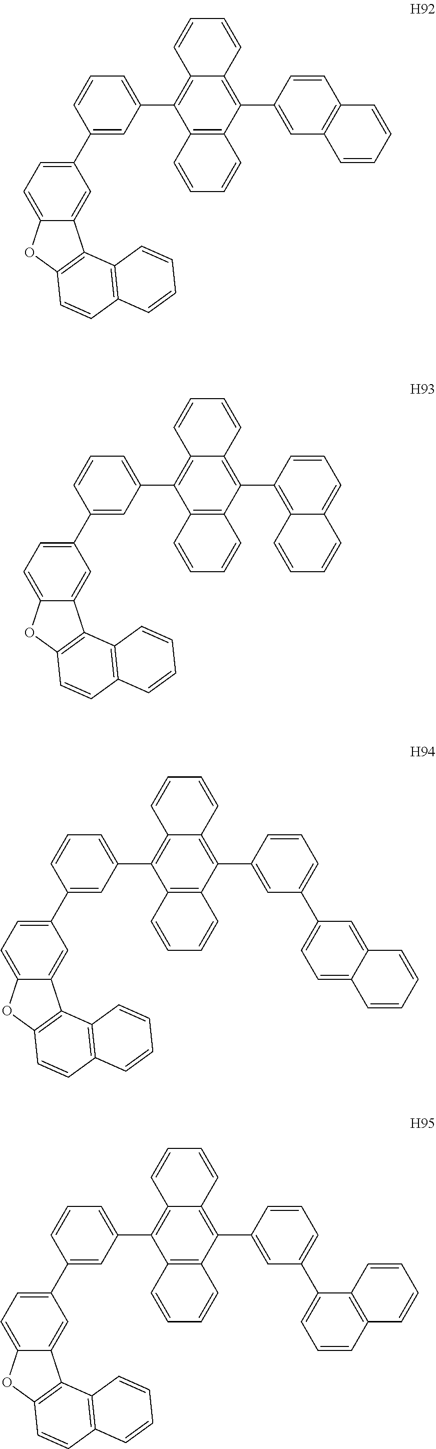 [SMILES: c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1]

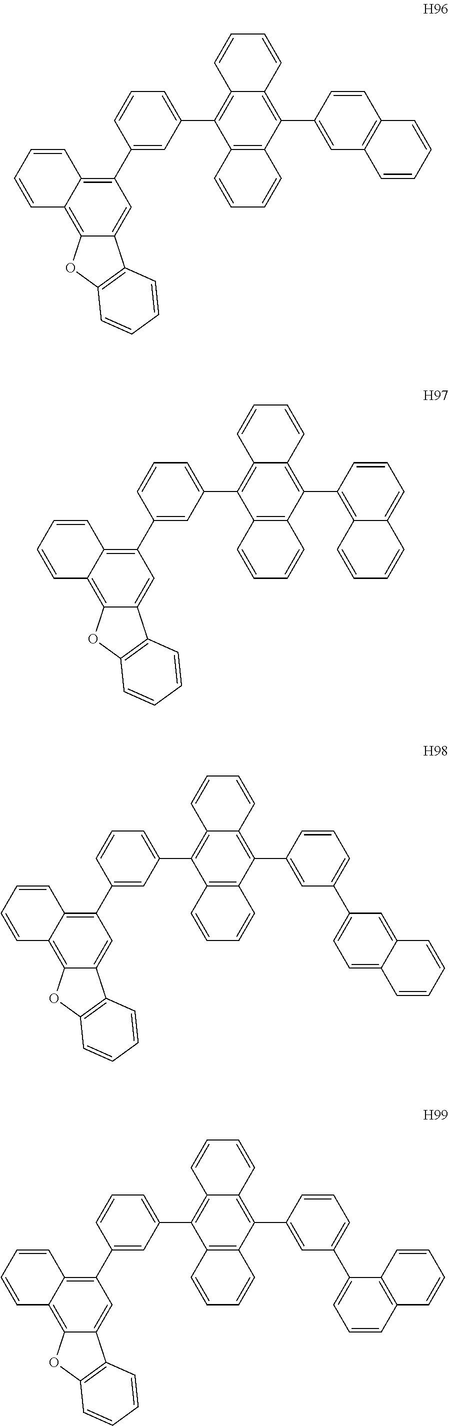 [SMILES: c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1]

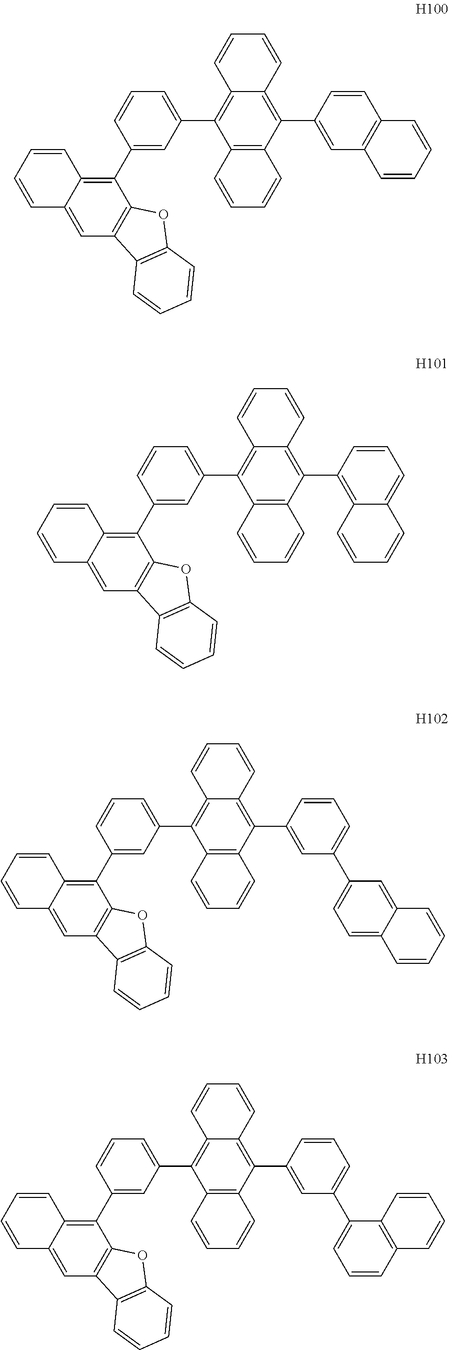 [SMILES: c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1]

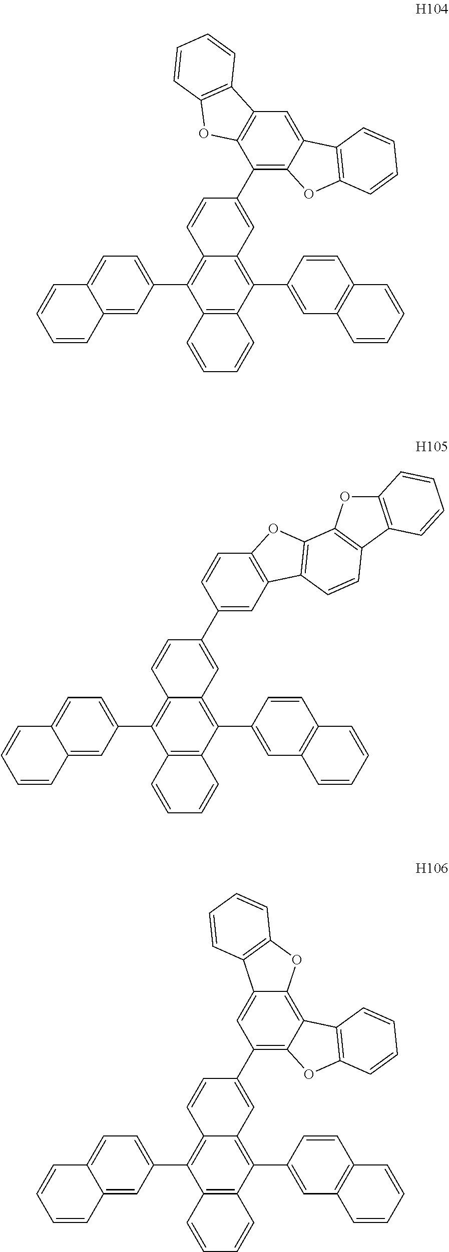 [SMILES: c1ccc2cc(-c3c4ccccc4c(-c4ccc5ccccc5c4)c4cc(-c5c6oc7ccccc7c6cc6c5oc5ccccc56)ccc34)ccc2c1 ; c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1]

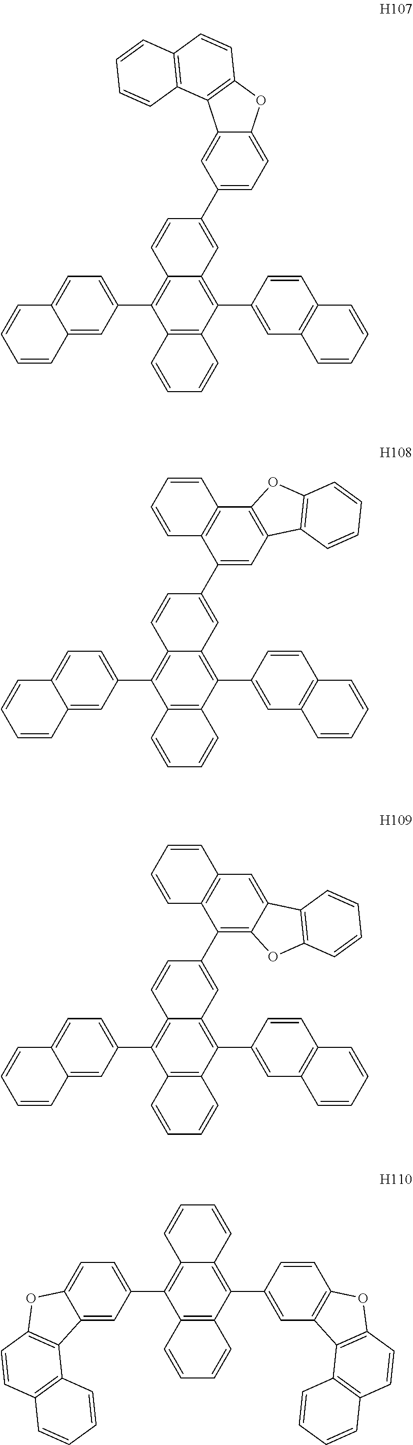 [SMILES: c1ccc2cc(-c3c4ccccc4c(-c4ccc5ccccc5c4)c4cc(-c5ccc6oc7ccc8ccccc8c7c6c5)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1 ; c1ccc2cc(-c3c4ccccc4c(-c4ccc5ccccc5c4)c4cc(-c5c6ccccc6cc6c5oc5ccccc56)ccc34)ccc2c1 ; c1ccc2c(c1)ccc1oc3ccc(-c4c5ccccc5c(-c5ccc6oc7ccc8ccccc8c7c6c5)c5ccccc45)cc3c12]

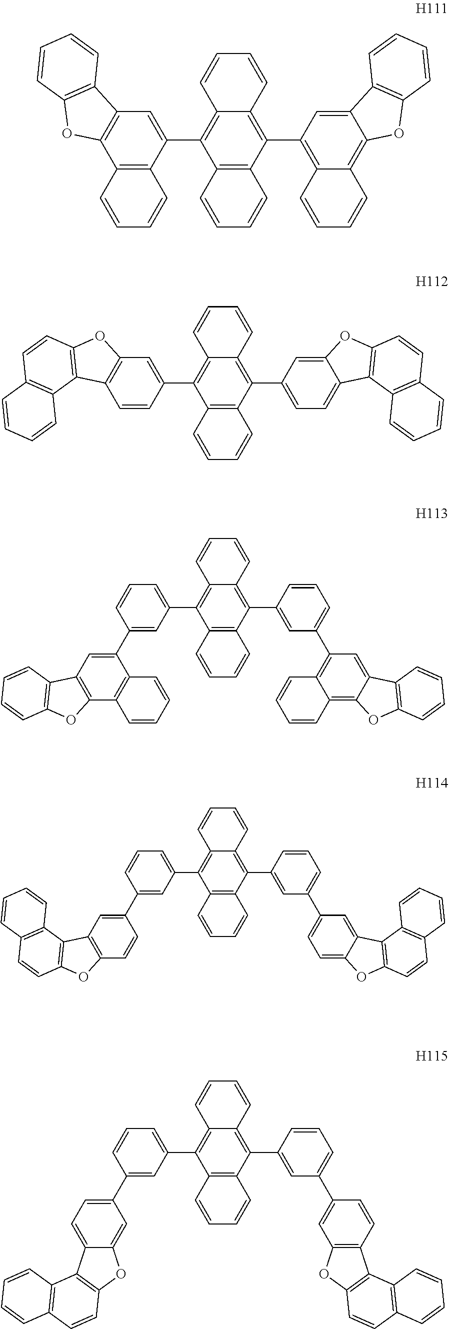 [SMILES: c1ccc2c(c1)oc1c3ccccc3c(-c3c4ccccc4c(-c4cc5c6ccccc6oc5c5ccccc45)c4ccccc34)cc21 ; c1ccc2c(c1)ccc1oc3cc(-c4c5ccccc5c(-c5ccc6c(c5)oc5ccc7ccccc7c56)c5ccccc45)ccc3c12 ; c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1]

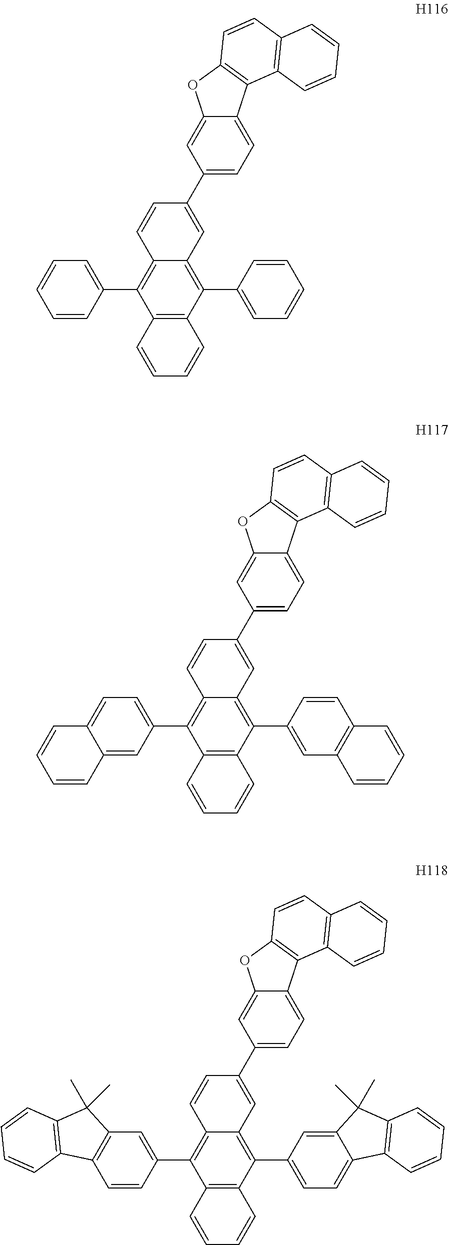 [SMILES: c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

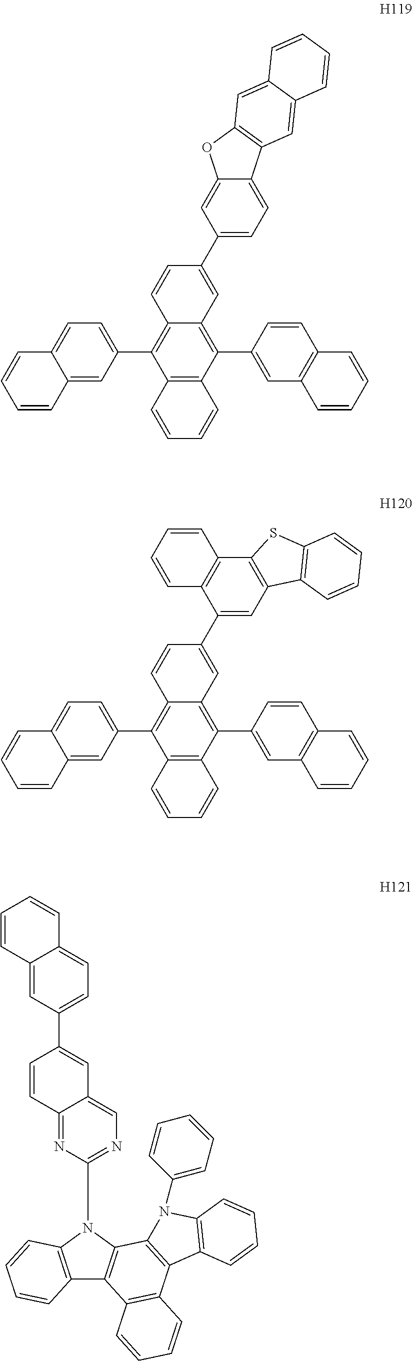 [SMILES: c1ccc2cc(-c3c4ccccc4c(-c4ccc5ccccc5c4)c4cc(-c5ccc6c(c5)oc5cc7ccccc7cc56)ccc34)ccc2c1 ; c1ccc2cc(-c3c4ccccc4c(-c4ccc5ccccc5c4)c4cc(-c5cc6c7ccccc7sc6c6ccccc56)ccc34)ccc2c1 ; c1ccc(-n2c3ccccc3c3c4ccccc4c4c5ccccc5n(-c5ncc6cc(-c7ccc8ccccc8c7)ccc6n5)c4c32)cc1]

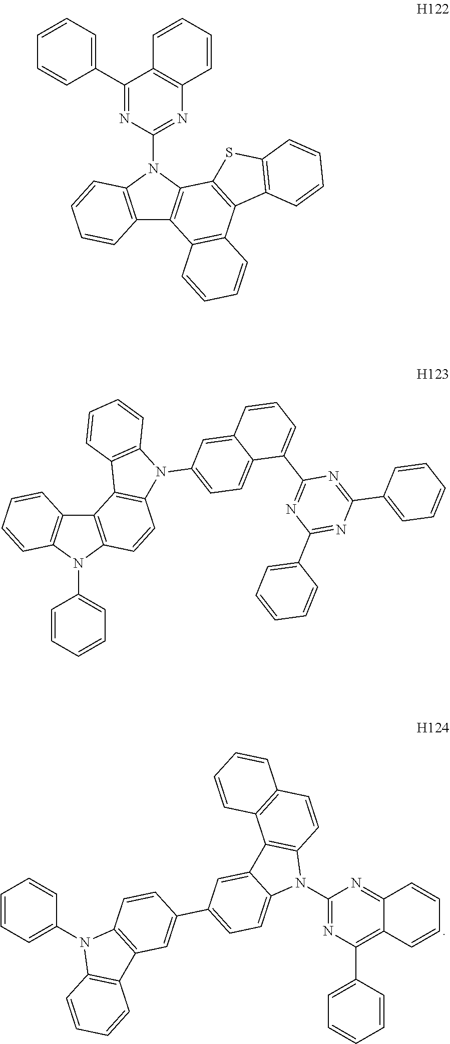 [SMILES: c1ccc(-c2nc(-n3c4ccccc4c4c5ccccc5c5c6ccccc6sc5c43)nc3ccccc23)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

### Phosphorescent Dopant

A phosphorescent dopant may include at least one transition metal as a central metal (e.g., a central metal atom).

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

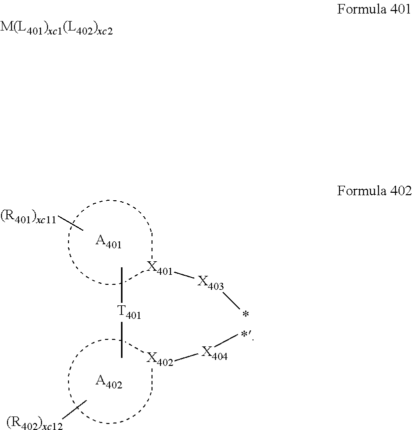

In Formulae 401 and 402,

M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),

L₄₀₁ may be a ligand represented by Formula 402, and xc1 may be 1, 2, or 3, wherein, when xc1 is 2 or more, two or more of L₄₀₁(s) may be identical to or different from each other,

L₄₀₂ may be an organic ligand, and xc2 may be 0, 1, 2, 3, or 4, wherein, when xc2 is 2 or more, two or more of L₄₀₂(s) may be identical to or different from each other,

X₄₀₁ and X₄₀₂ may each independently be nitrogen or carbon,

ring A₄₀₁ and ring A₄₀₂ may each independently be a C₃-C₆₀ carbocyclic group or a C₁-C₆₀ heterocyclic group,

T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)═C(Q₄₁₂)-*′, *—C(Q₄₁₁)=*′, or *═C═*′,

X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),

Q₄₁₁ to Q₄₁₄ may each independently be the same as described in connection with Q₁ (to be described in more detail below),

R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),

Q₄₀₁ to Q₄₀₃ may each independently be the same as described in connection with Qi,

xc11 and xc12 may each independently be an integer from 0 to 10, and

* and *′ in Formula 402 each indicate a binding site to M in Formula 401.

In an embodiment, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 401 is 2 or more, two ring A₄₀₁(s) in two or more of L₄₀₁ (s) may optionally be linked to each other via T₄₀₂, which is a linking group, or two ring A₄₀₂(s) in two or more of L₄₀₁ (s) may optionally be linked to each other via T₄₀₃, which is a linking group (see Compounds PD1 to PD4 and PD7). T₄₀₂ and T₄₀₃ may each independently be the same as described in connection with T₄₀₁

L₄₀₂ in Formula 401 may be an organic ligand. For example, L₄₀₂ may be a halogen group, a diketone group (for example, an acetylacetonate group), a carboxylic acid group (for example, a picolinate group), —C(═O), an isonitrile group, a —CN group, a phosphorus group (for example, a phosphine group or a phosphite group), or any combination thereof.

The phosphorescent dopant may include, for example, one of Group P1 (i.e., one compound in Group P1), one of Group P2 (i.e., one compound in Group P2), or any combination thereof:

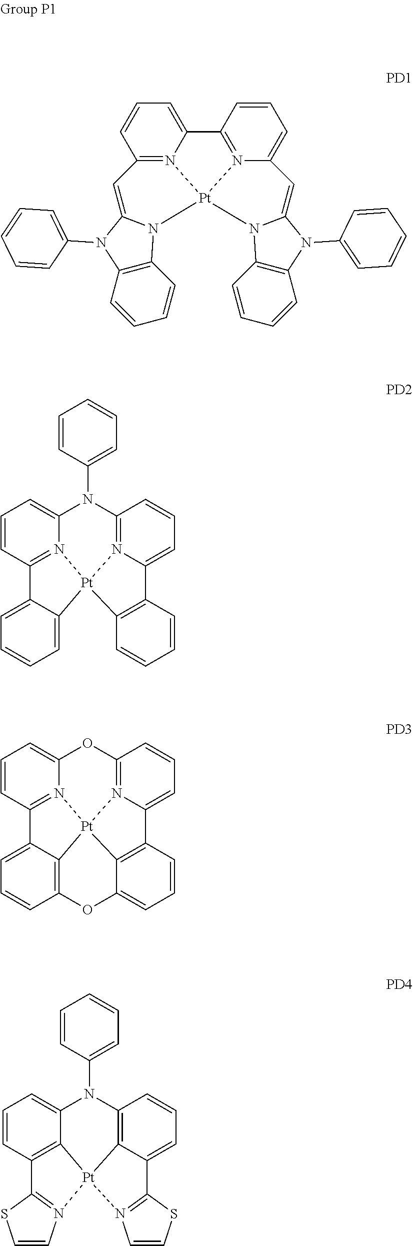

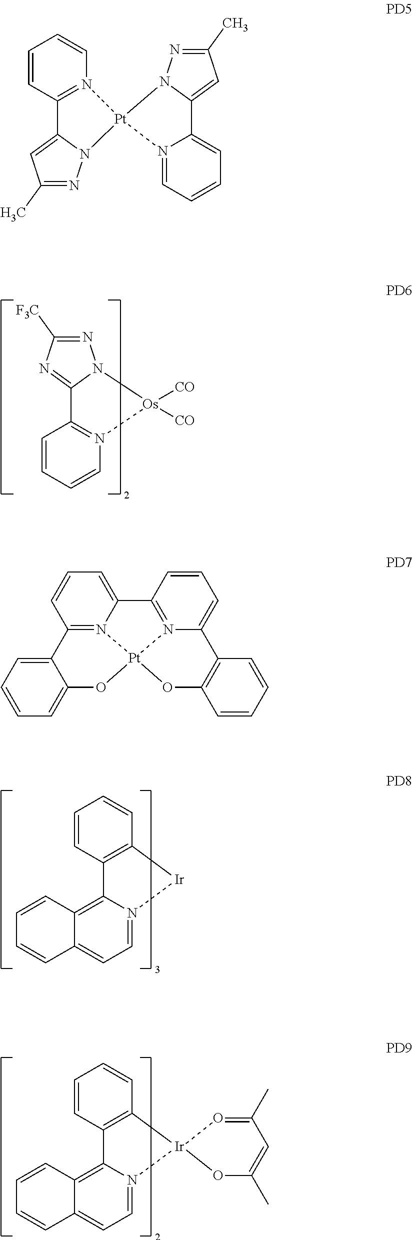

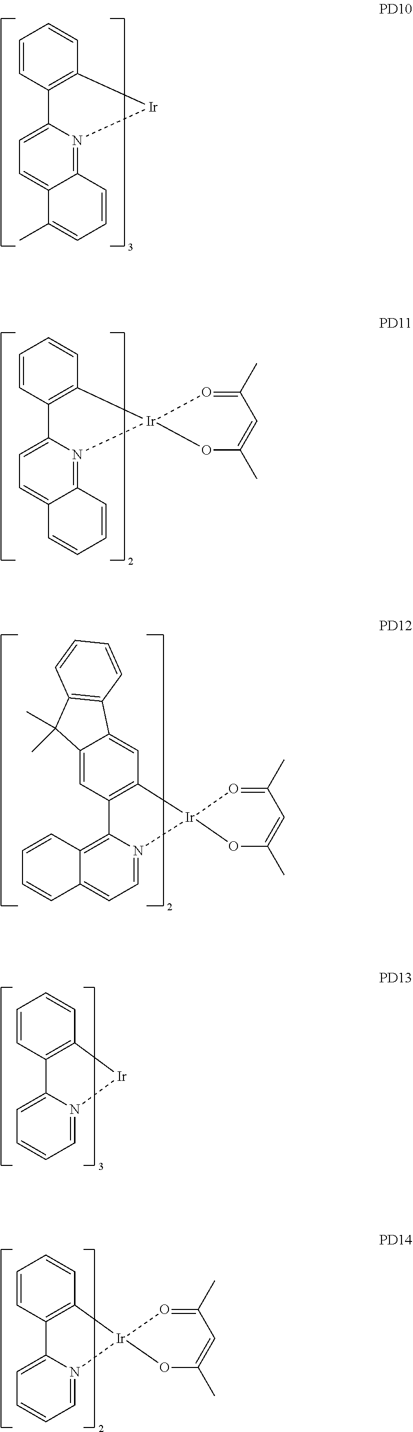

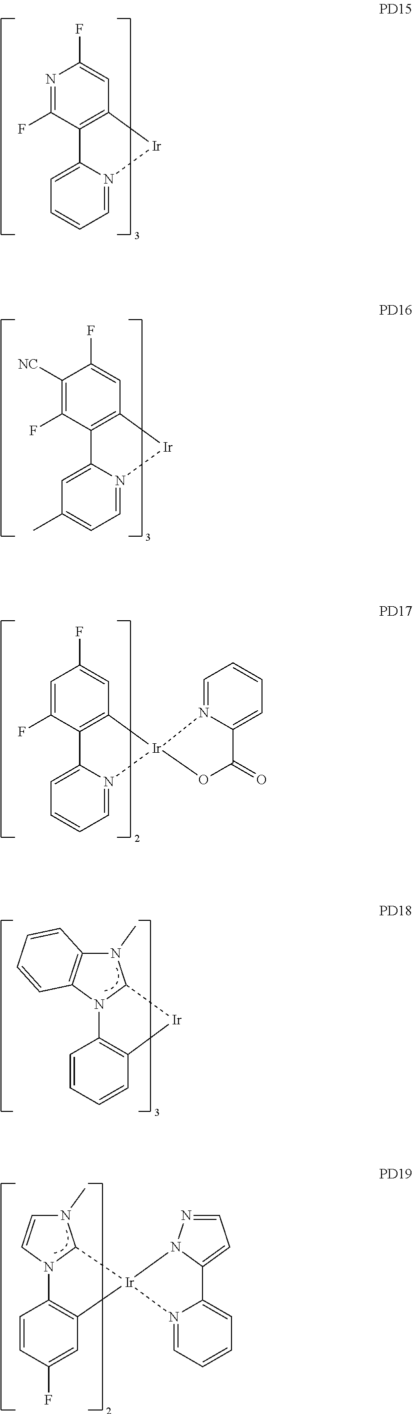

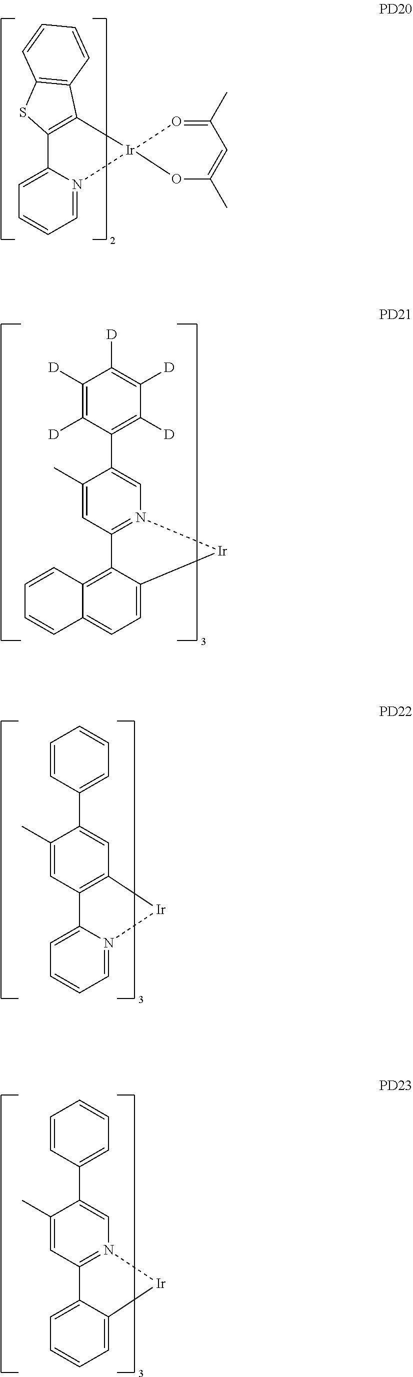

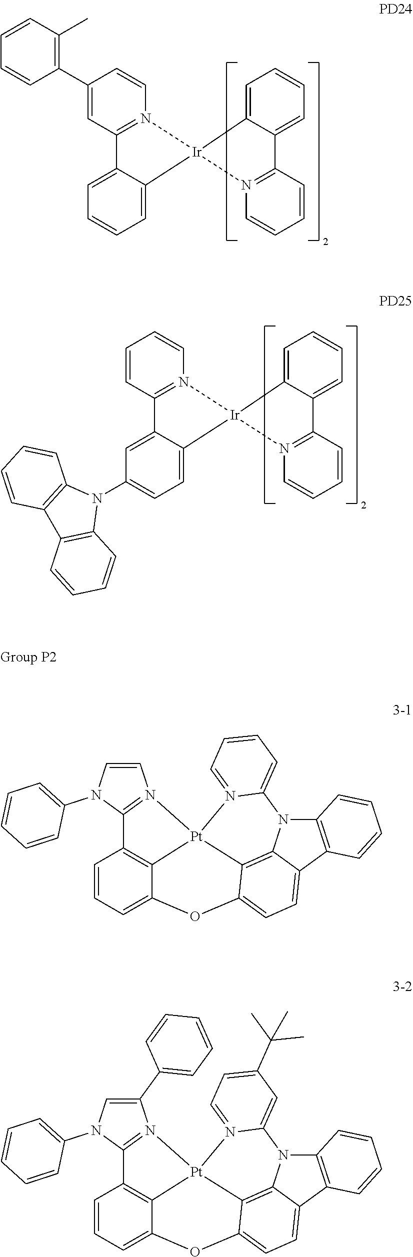

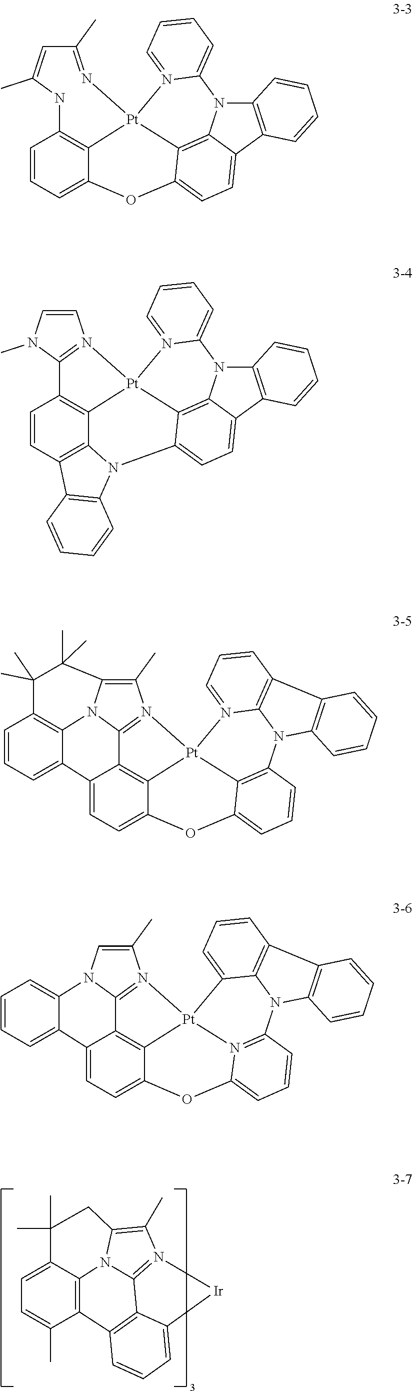

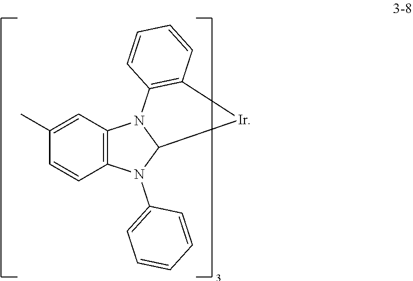

### Fluorescent Dopant

A fluorescent dopant may include an amine group-containing compound, a styryl group-containing compound, or any combination thereof.

For example, the fluorescent dopant may include a compound represented by Formula 501:

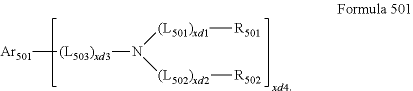

In Formula 501,

Ar₅₀₁, L₅₀₁ to L₅₀₃, R₅₀₁, and R₅₀₂ may each independently be a C₅-C₆₀ carbocyclic group unsubstituted or substituted with at least one R₁₀ₐ or a C₁-C₆₀ heterocyclic group unsubstituted or substituted with at least one R₁₀ₐ,

xd1 to xd3 may each independently be 0, 1, 2, or 3, and

xd4 may be 1, 2, 3, 4, 5, or 6.

In an embodiment, Ar₅₀₁ in Formula 501 may be a condensed cyclic group (for example, an anthracene group, a chrysene group, a pyrene group, etc.) in which three or more monocyclic groups are condensed with one another.

In one or more embodiments xd4 in Formula 501 may be 2.

For example, the fluorescent dopant may include one of Group F1 (i.e., one compound in Group F1), one of Group F2 (i.e., one compound in Group F2), DPVBi, DPAVBi, or any combination thereof:

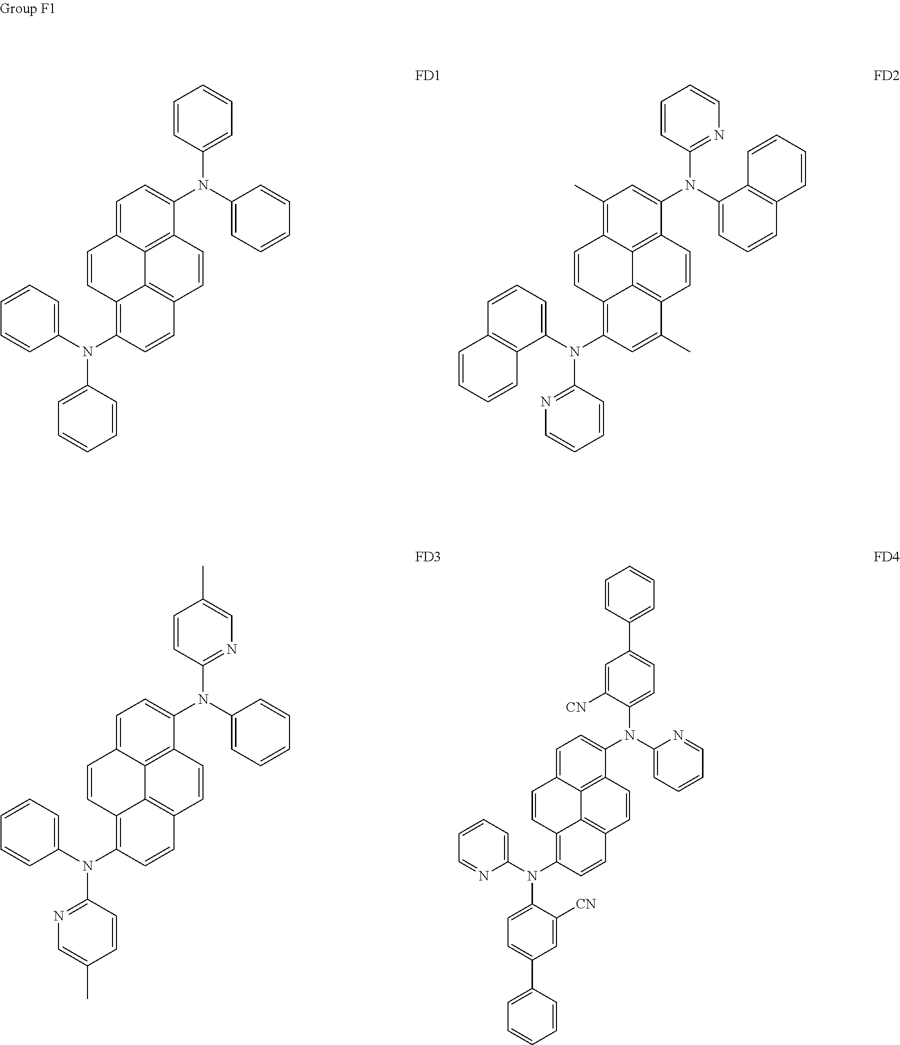

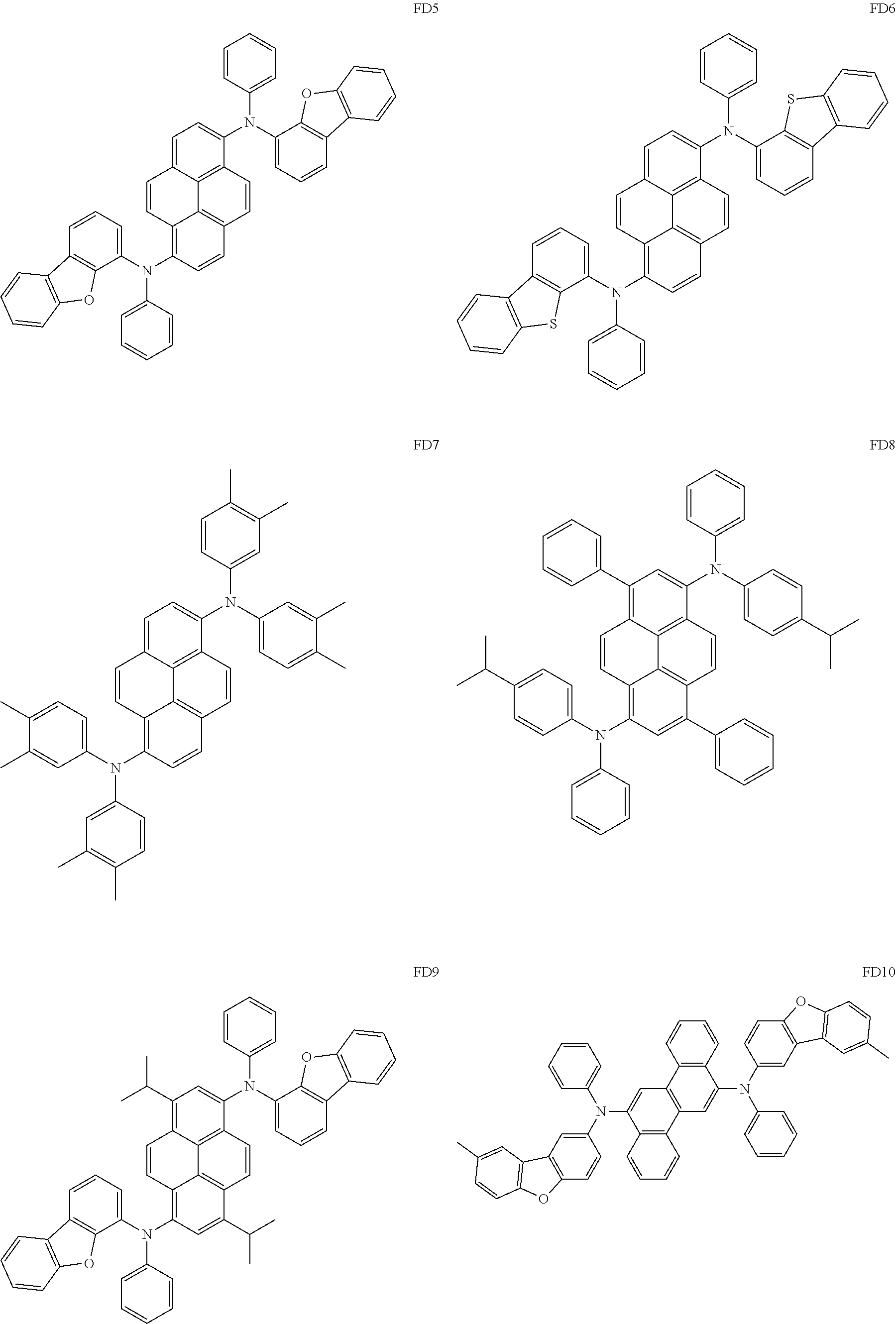

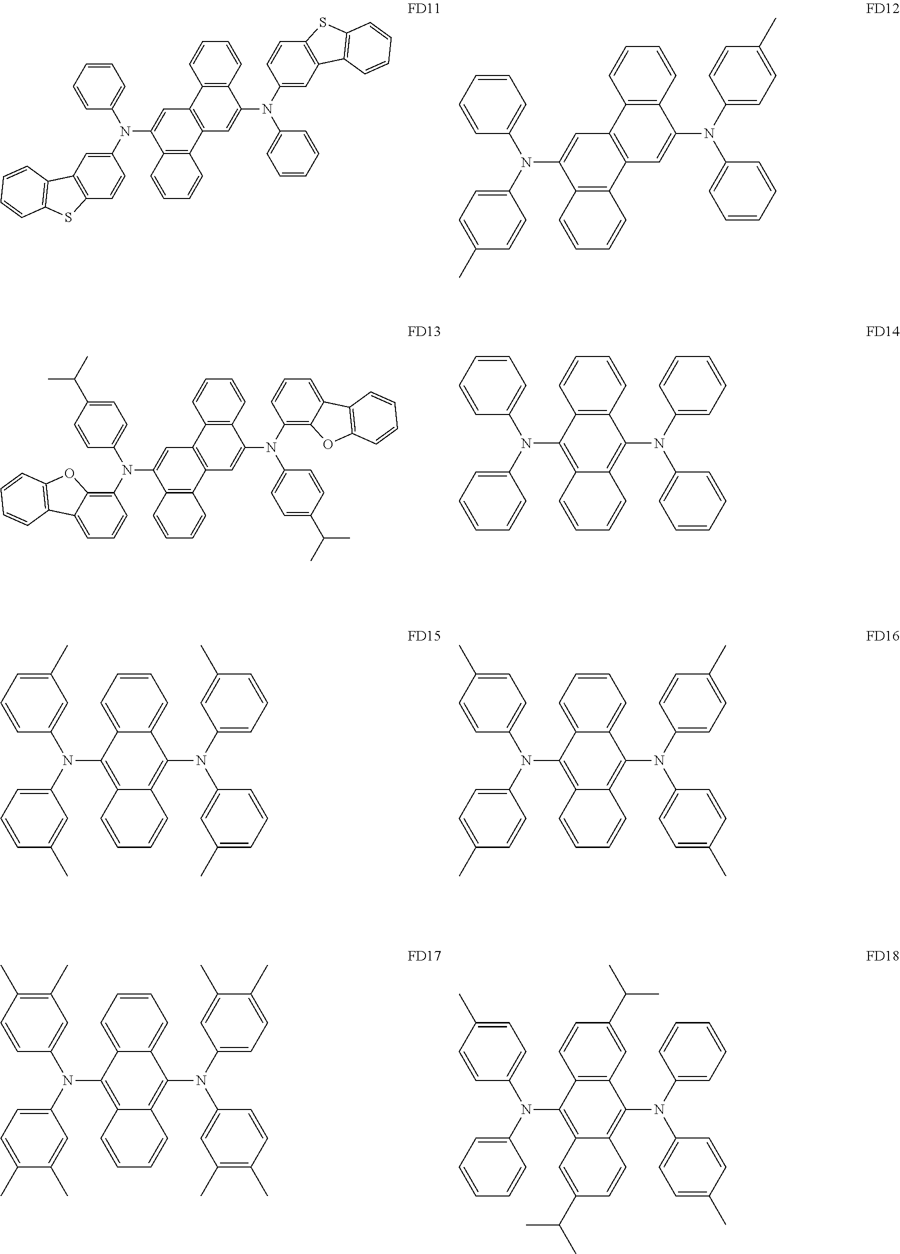

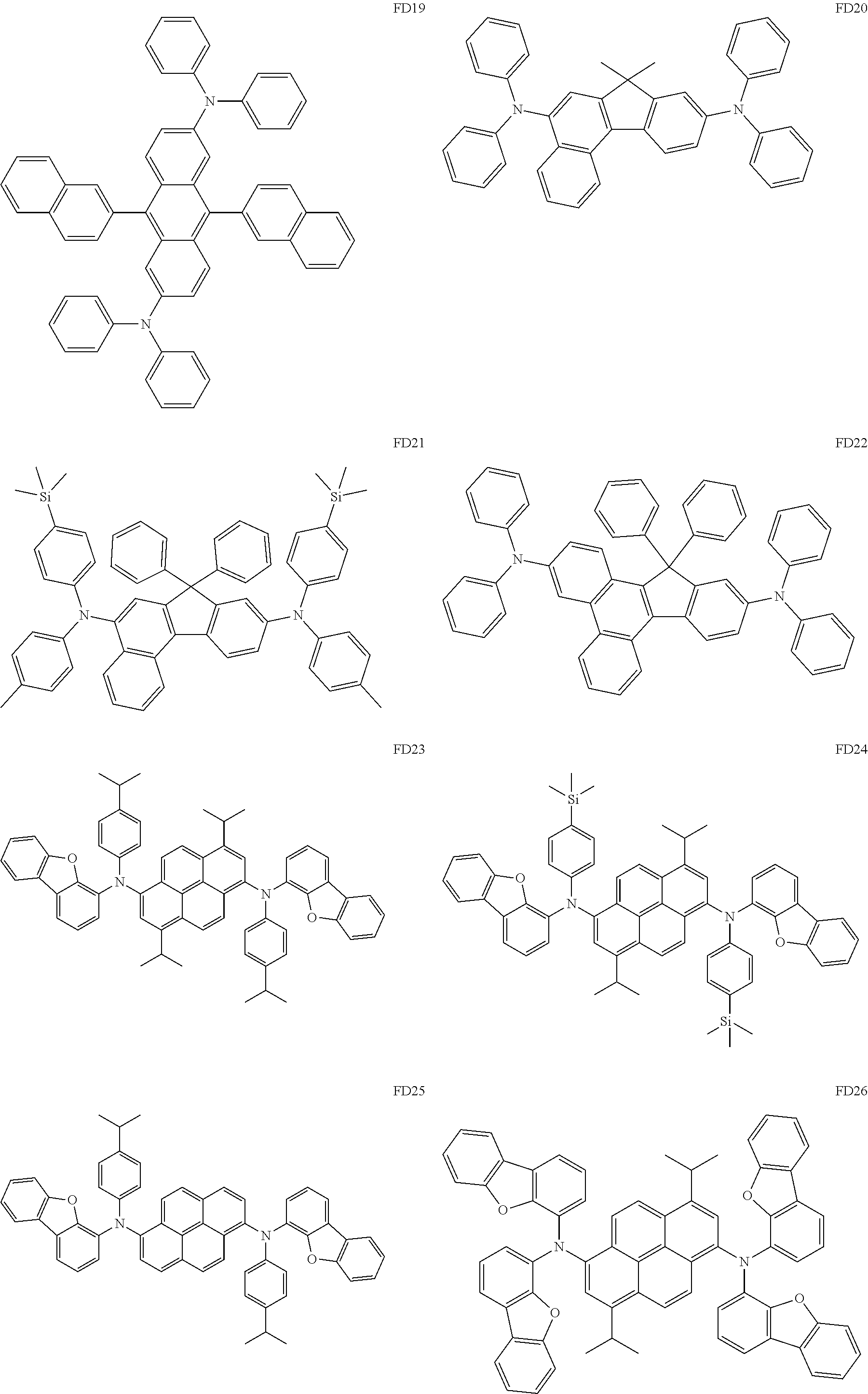

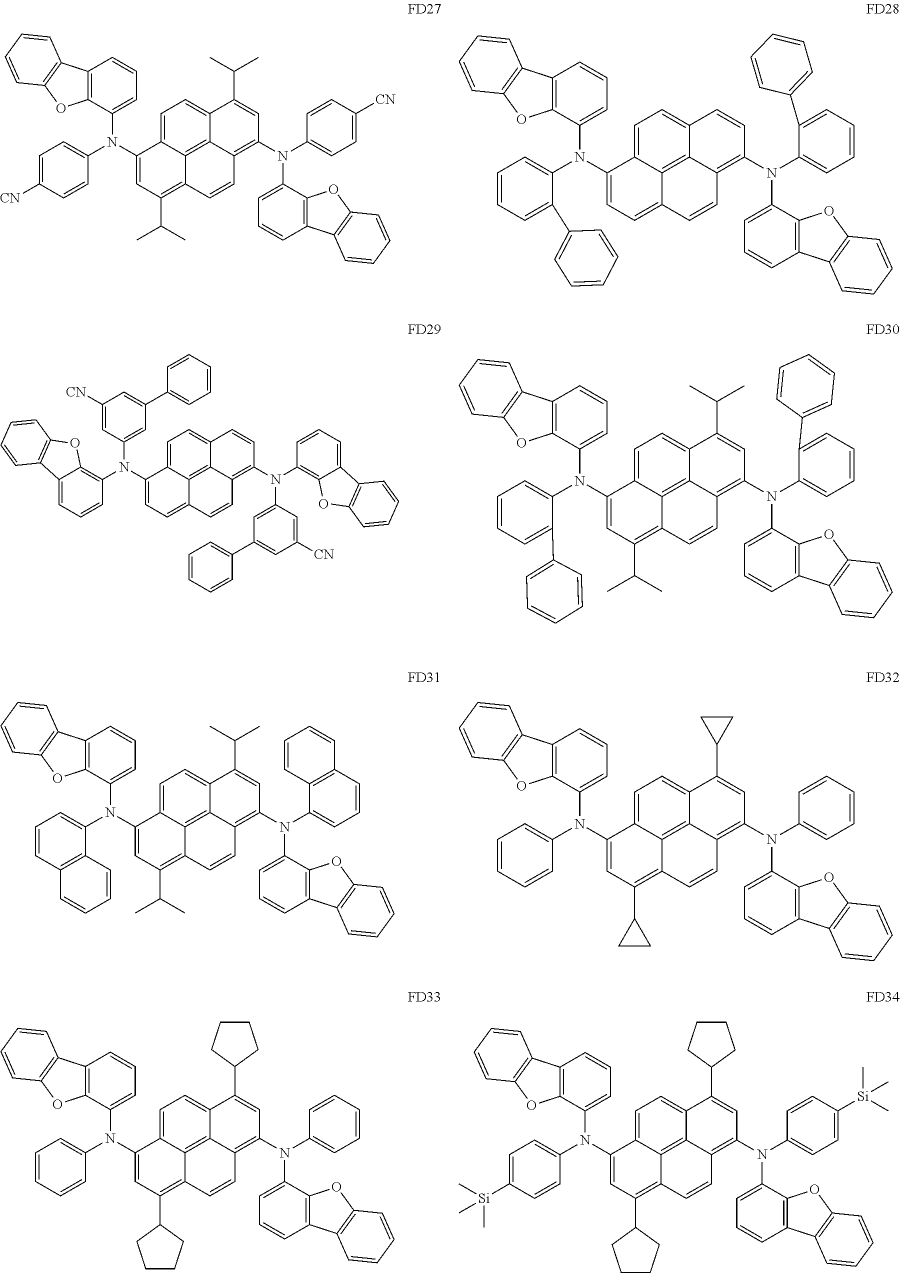

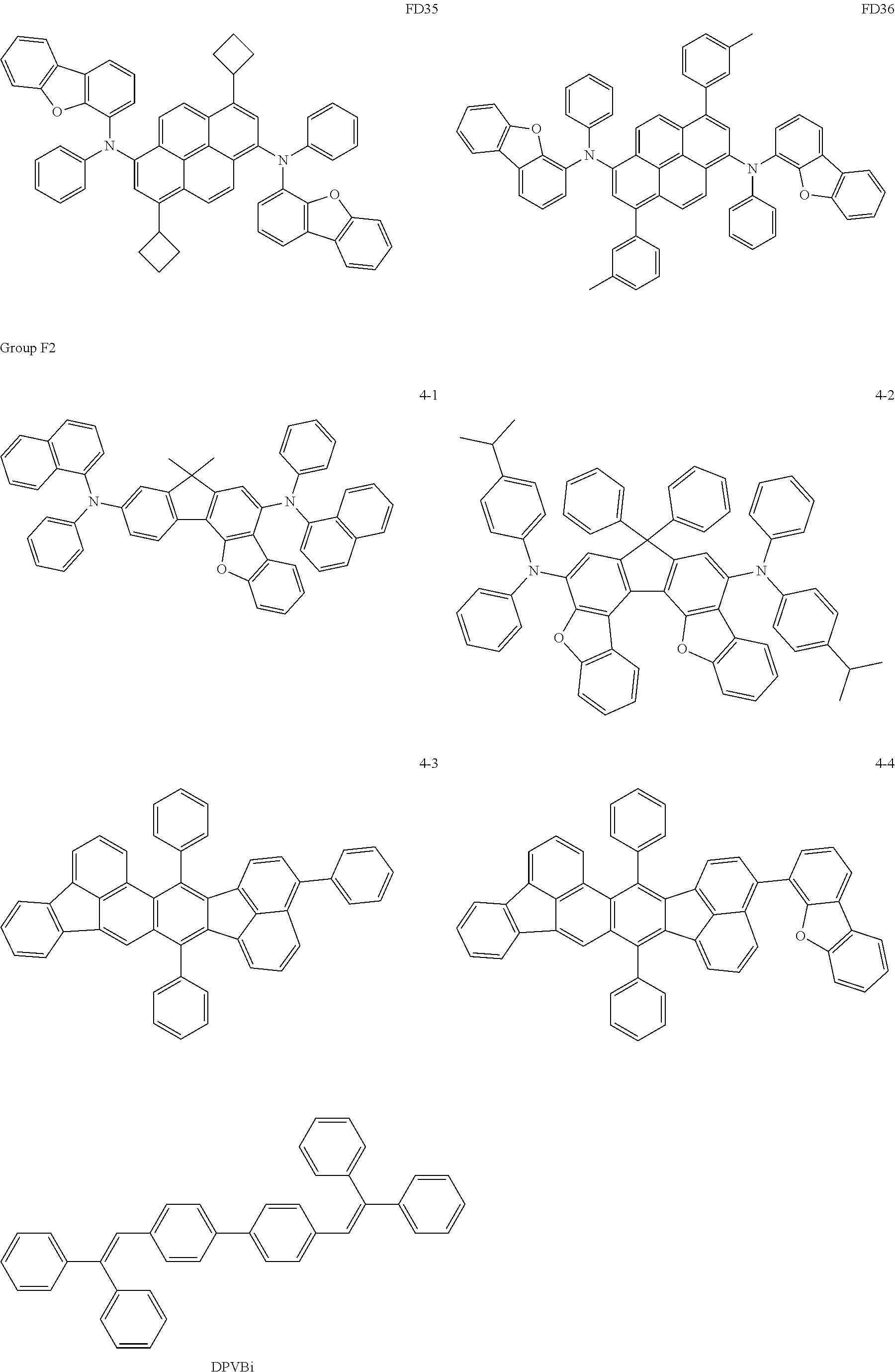

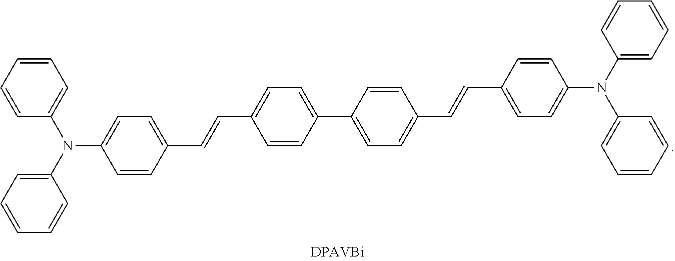 [SMILES: C(=C\c1ccc(N(c2ccccc2)c2ccccc2)cc1)/c1ccc(-c2ccc(/C=C/c3ccc(N(c4ccccc4)c4ccccc4)cc3)cc2)cc1]

### Delayed Fluorescence Material

The emission layer may include a delayed fluorescence material.

The delayed fluorescence material may be selected from any compound capable of emitting delayed fluorescence according to a delayed fluorescence emission mechanism.

The delayed fluorescence material included in the emission layer may serve as a host or a dopant, depending on a kind (e.g., type) of other materials included in the emission layer.

In an embodiment, a difference between a triplet energy level (eV) of the delayed fluorescence material and a singlet energy level (eV) of the delayed fluorescence material may be equal to or greater than 0 eV and equal to or less than 0.5 eV. When the difference between the triplet energy level (eV) of the delayed fluorescence material and the singlet energy level (eV) of the delayed fluorescence material is within the range above, the up-conversion from the triplet state to the singlet state in the delayed fluorescence material may be effectively performed, thereby improving luminescence efficiency of the light-emitting device 10.

For example, the delayed fluorescence material may include i) a material including at least one electron donor (for example, a π electron-rich C₃-C₆₀ cyclic group, such as a carbazole group, etc.) and at least one electron acceptor (for example, a sulfoxide group, a cyano group, a π electron-deficient nitrogen-containing C₁-C₆₀ cyclic group, etc.), and/or ii) a material including a C₈-C₆₀ polycyclic group including two or more cyclic groups condensed while sharing boron (B).

Non-limiting examples of the delayed fluorescence material are one of Group D1 (i.e., one compound in Group D1), one of Group D2 (i.e., one compound in Group D2), or any combination thereof:

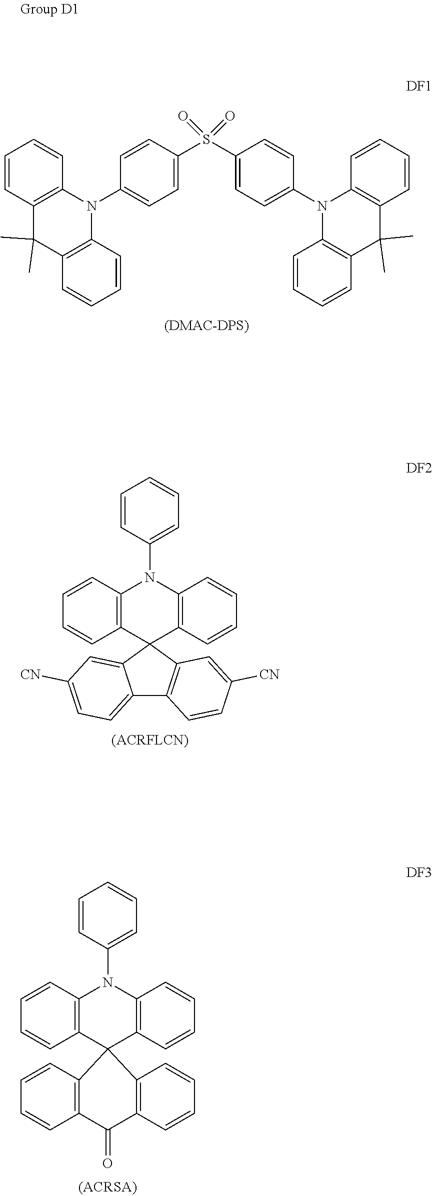

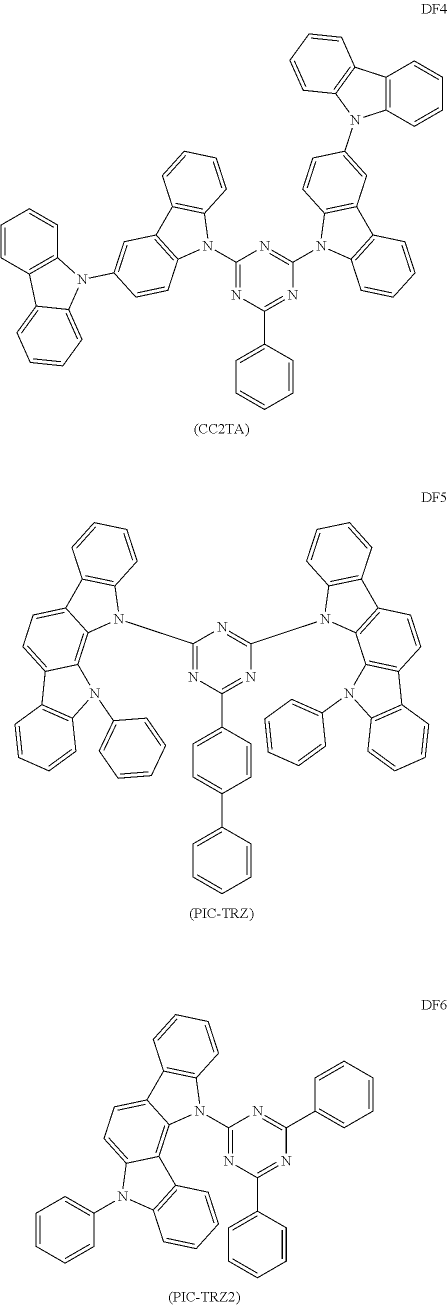 [SMILES: c1ccc(-c2nc(-n3c4ccccc4c4cc(-n5c6ccccc6c6ccccc65)ccc43)nc(-n3c4ccccc4c4cc(-n5c6ccccc6c6ccccc65)ccc43)n2)cc1 ; c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1]

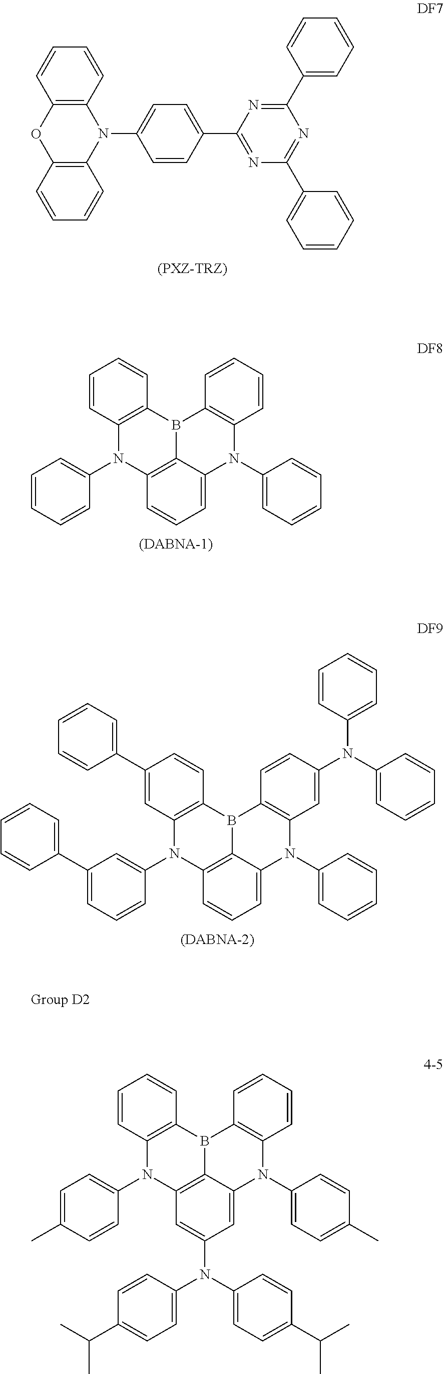

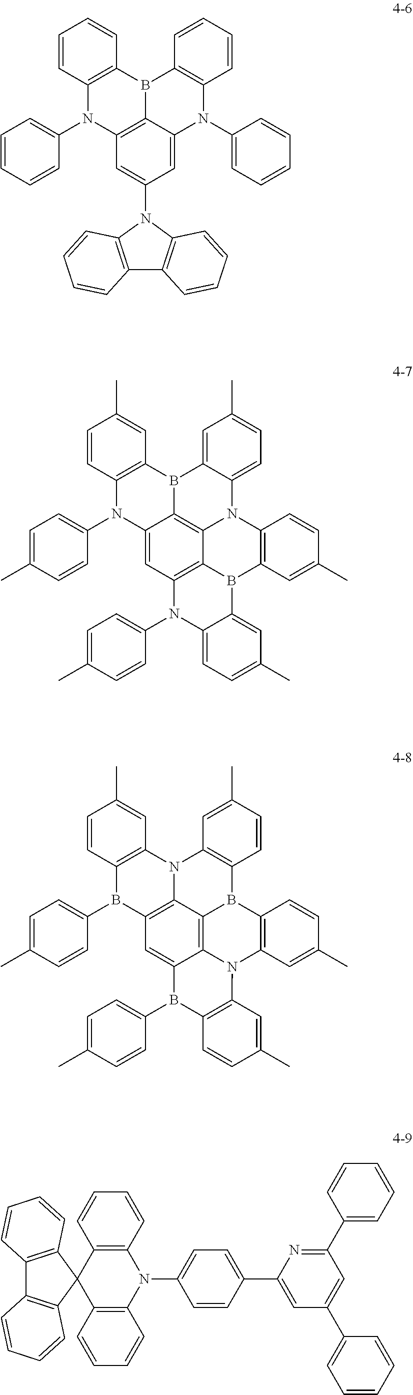

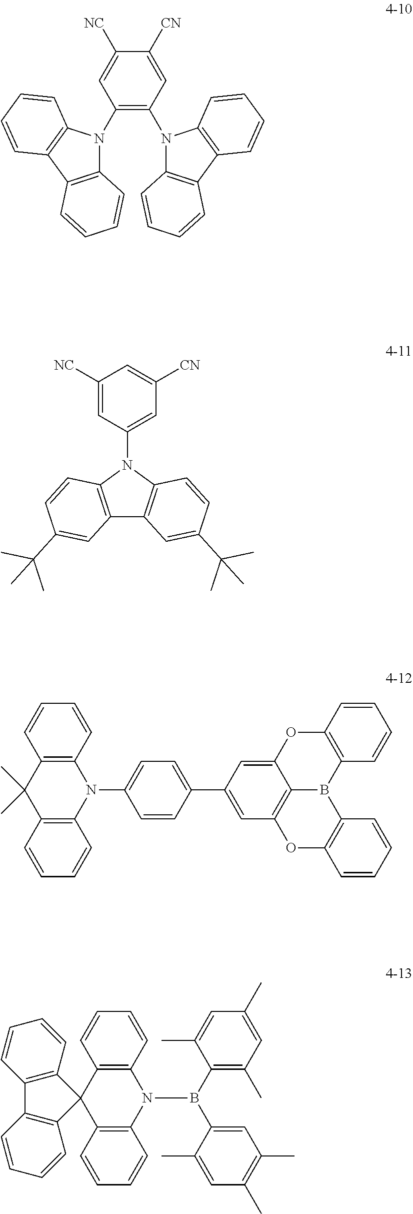

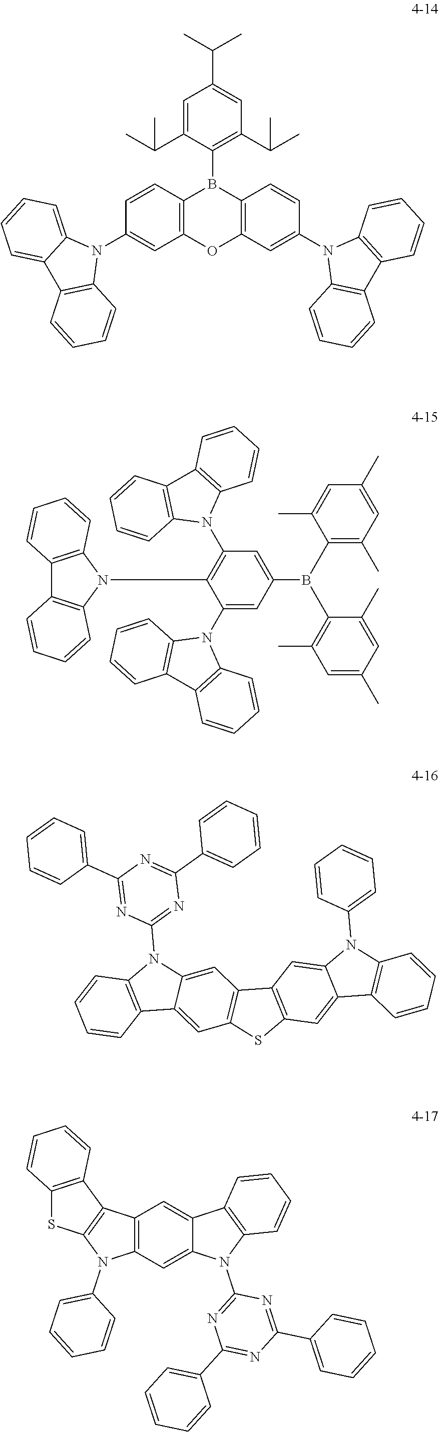 [SMILES: CC(C)c1cc(C(C)C)c(B2c3ccc(-n4c5ccccc5c5ccccc54)cc3Oc3cc(-n4c5ccccc5c5ccccc54)ccc32)c(C(C)C)c1 ; Cc1cc(C)c(B(c2cc(-n3c4ccccc4c4ccccc43)c(-n3c4ccccc4c4ccccc43)c(-n3c4ccccc4c4ccccc43)c2)c2c(C)cc(C)cc2C)c(C)c1 ; c1ccc(-c2nc(-c3ccccc3)nc(-n3c4ccccc4c4cc5sc6cc7c8ccccc8n(-c8ccccc8)c7cc6c5cc43)n2)cc1 ; c1ccc(-c2nc(-c3ccccc3)nc(-n3c4ccccc4c4cc5c6c7ccccc7sc6n(-c6ccccc6)c5cc43)n2)cc1]

### Quantum Dot

The emission layer may include a quantum dot.

The term “quantum dot” as used herein refers to a crystal of a semiconductor compound, and the quantum dot may include any suitable material emitting various suitable emission wavelengths according to the size of the crystal.

A diameter of the quantum dot may be, for example, in a range of about 1 nm to about 10 nm.

The quantum dot may be synthesized by a wet chemical process, an organometallic chemical vapor deposition process, a molecular beam epitaxy process, or a similar process.

According to the wet chemical process, an organic solvent and a precursor material are mixed to grow a quantum dot crystal (e.g., a particle of quantum dot crystal). When the crystal grows, the organic solvent acts as a dispersant naturally coordinated on the surface of the quantum dot crystal and controls the growth of the crystal. Accordingly, through a process that is easily performed at low costs compared to a vapor deposition process (such as a metal organic chemical vapor deposition

(MOCVD) process and/or a molecular beam epitaxy (MBE) process), the growth of quantum dot particles may be controlled.

The quantum dot may include a Groups III-VI semiconductor compound; a Groups II-VI semiconductor compound; a Groups III-V semiconductor compound; a Groups semiconductor compound; a Groups IV-VI semiconductor compound; a

Group IV element or compound; or any combination thereof.

Non-limiting examples of the Groups III-VI semiconductor compound are: a binary compound, such as In₂S₃; a ternary compound, such as AgInS, AgInS₂, CuInS, CuInS₂, and/or the like; or any combination thereof.

Non-limiting examples of the Group II-VI semiconductor compound may include: a binary compound, such as CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or any combination thereof.

Non-limiting examples of the Groups III-V semiconductor compound are: a binary compound, such as GaN, GaP, GaAs, GaSb, AlN, AlP, AlAs, AlSb, InN, InP, InAs, InSb, and/or the like; a ternary compound, such as GaNP, GaNAs, GaNSb, GaPAs, GaPSb, AINP, AINAs, AINSb, AIPAs, AIPSb, InGaP, InNP, InAIP, InNAs, InNSb, InPAs, InPSb, and/or the like; a quaternary compound, such as GaAINP, GaAINAs, GaAINSb, GaAIPAs, GaAIPSb, GaInNP, GaInNAs, GaInNSb, GaInPAs, GaInPSb, InAINP, InAINAs, InAINSb, InAIPAs, InAIPSb, and/or the like; or any combination thereof. In an embodiment, the Groups III-V semiconductor compound may further include a Group II element. Non-limiting examples of the Groups III-V semiconductor compound further including the Group II element are InZnP, InGaZnP, InAlZnP, and/or the like.

Non-limiting examples of the Groups III-VI semiconductor compound are: a binary compound, such as GaS, GaSe, Ga₂Se₃, GaTe, InS, InSe, In₂Se₃, InTe, and/or the like; a ternary compound, such as InGaS₃, InGaSe₃, and/or the like; or any combination thereof.

Non-limiting examples of the Groups semiconductor compound are: a ternary compound, such as AgInS, AgInS₂, CuInS, CuInS₂, CuGaO₂, AgGaO₂, AgAlO₂, and/or the like; or any combination thereof.

Non-limiting examples of the Groups IV-VI semiconductor compound are: a binary compound, such as SnS, SnSe, SnTe, PbS, PbSe, PbTe, and/or the like; a ternary compound, such as SnSeS, SnSeTe, SnSTe, PbSeS, PbSeTe, PbSTe, SnPbS, SnPbSe, SnPbTe, and/or the like; a quaternary compound, such as SnPbSSe, SnPbSeTe, SnPbSTe, and/or the like; or any combination thereof.

Non-limiting examples of the Group IV element or compound are: a single element compound, such as Si, Ge, and/or the like; a binary compound, such as SiC, SiGe, and/or the like; or any combination thereof.

Each element included in a multi-element compound, such as the binary compound, the ternary compound, and/or the quaternary compound, may be present in particles at a uniform concentration or a non-uniform concentration.

In one embodiment, the quantum dot may have a single structure having a uniform concentration of each element included in the corresponding quantum dot or a dual structure of a core-shell. For example, a material included in the core may be different from a material included in the shell.

The shell of the quantum dot may serve as a protective layer for maintaining semiconductor characteristics by preventing or reducing chemical degeneration of the core and/or may serve as a charging layer for imparting electrophoretic characteristics to the quantum dot. The shell may be a single layer or a multilayer. An interface between the core and the shell may have a concentration gradient in which the concentration of elements existing in the shell decreases toward the center.

Non-limiting examples of the shell of the quantum dot may include a metal oxide or a non-metal oxide, a semiconductor compound, or any combination thereof. Non-limiting examples of the metal oxide or non-metal oxide are: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, and/or the like; a ternary compound, such as MgAl₂O₄, CoFe₂O₄, NiFe₂O₄, CoMn₂O₄, and/or the like; or any combination thereof. Non-limiting examples of the semiconductor compound are, as described above, a Groups III-VI semiconductor compound; a Groups II-VI semiconductor compound; a Groups III-V semiconductor compound; a Groups III-VI semiconductor compound; a Groups I-III-VI semiconductor compound; a Groups IV-VI semiconductor compound; or any combination thereof. Non-limiting examples of the semiconductor compound are CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width of half maximum (FWHM) of an emission wavelength spectrum of the quantum dot may be equal to or less than about 45 nm, for example, equal to or less than about 40 nm, or for example, equal to or less than about 30 nm. When the FWHM of the emission wavelength spectrum of the quantum dot is within these ranges, color purity or color reproduction may be improved. In addition, light emitted through such a quantum dot may be irradiated in omnidirection (e.g., in all directions), thereby improving a wide viewing angle.

In addition, the quantum dot may be a spherical, pyramidal, multi-arm, or cubic nanoparticle, a nanotube, a nanowire, a nanofiber, or a nanoplate particle.

By adjusting the size of the quantum dot, the energy band gap may also be adjusted, thereby obtaining light of various suitable wavelengths in the quantum dot emission layer. Therefore, by utilizing quantum dots of different sizes, a light-emitting device that emits light of various suitable wavelengths may be implemented. In one embodiment, the size of the quantum dot may be selected to emit red, green and/or blue light. In addition, the size of the quantum dot may be configured to allow combining light of various suitable colors, so as to emit white light.

### Electron Transport Region in Interlayer 130

The electron transport region may have: i) a single-layered structure consisting of a single layer consisting of a single material, ii) a single-layered structure consisting of a single layer including (e.g., consisting of) a plurality of different materials, or iii) a multi-layered structure including a plurality of layers including different materials.

The electron transport region may include a buffer layer, a hole blocking layer, an electron control layer, an electron transport layer, an electron injection layer, or any combination thereof.

For example, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein for each structure, constituting layers are sequentially stacked on the emission layer in the respective stated order.

For example, the second layer may be a buffer layer, a hole blocking layer, or an electron control layer, and the fourth layer may be an electron transport layer. However, embodiments of the present disclosure are not limited thereto.

The electron transport region (for example, the buffer layer, the hole blocking layer, the electron control layer, or the electron transport layer in the electron transport region) may include a metal-free compound including at least one π electron-deficient nitrogen-containing C₁-C₆₀ cyclic group.

For example, the electron transport region may include a compound represented by Formula 601:

[Ar₆₀₁]ₓₑ₁₁-[(L₆₀₁)ₓₑ₁-R₆₀₁]ₓₑ₂₁ Formula 601

wherein, in Formula 601,

Ar₆₀₁ and L₆₀₁ may each independently be a C₅-C₆₀ carbocyclic group unsubstituted or substituted with at least one R₁₀ₐ or a C₁-C₆₀ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ may each independently be the same as described in connection with Qi,

xe21 may be 1, 2, 3, 4, or 5, and

at least one of Ar₆₀₁, L₆₀₁, or R₆₀₁ may (e.g., each independently) be a π electron-deficient nitrogen-containing C₁-C₆₀ cyclic group unsubstituted or substituted with at least one R₁₀ₐ.

In an embodiment, when xe11 in Formula 601 is 2 or more, two or more of Ar₆₀₁(s) may be linked to each other via a single bond.

In one or more embodiments, Ar₆₀₁ in Formula 601 may be a substituted or unsubstituted anthracene group.

In one or more embodiments, the electron transport region may include a compound represented by Formula 601-1:

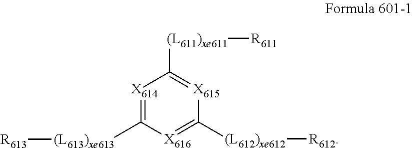

In Formula 601-1,

X₆₁₄ may be N or C(R₆₁₄), X₆₁₅ may be N or C(R₆₁₅), X₆₁₆ may be N or

C(R₆₁₆), and at least one of X₆₁₄ to X₆₁₆ may be N,

L₆₁₁ to L₆₁₃ may each independently be the same as described in connection with L₆₀₁,

xe611 to xe613 may each independently be the same as described in connection with xe1

R₆₁₁ to R₆₁₃ may each independently be the same as described in connection with R₆₀₁, and

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

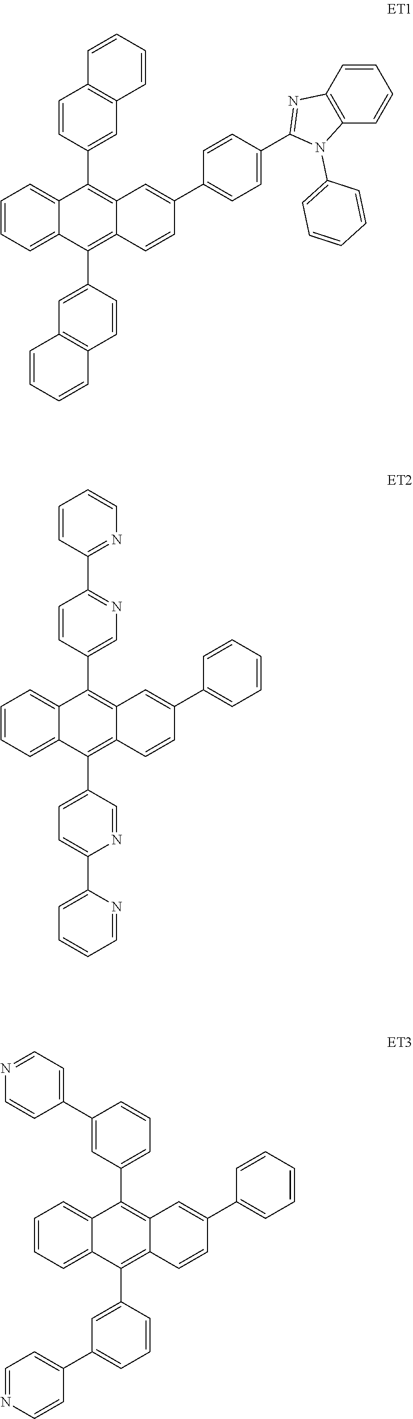 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

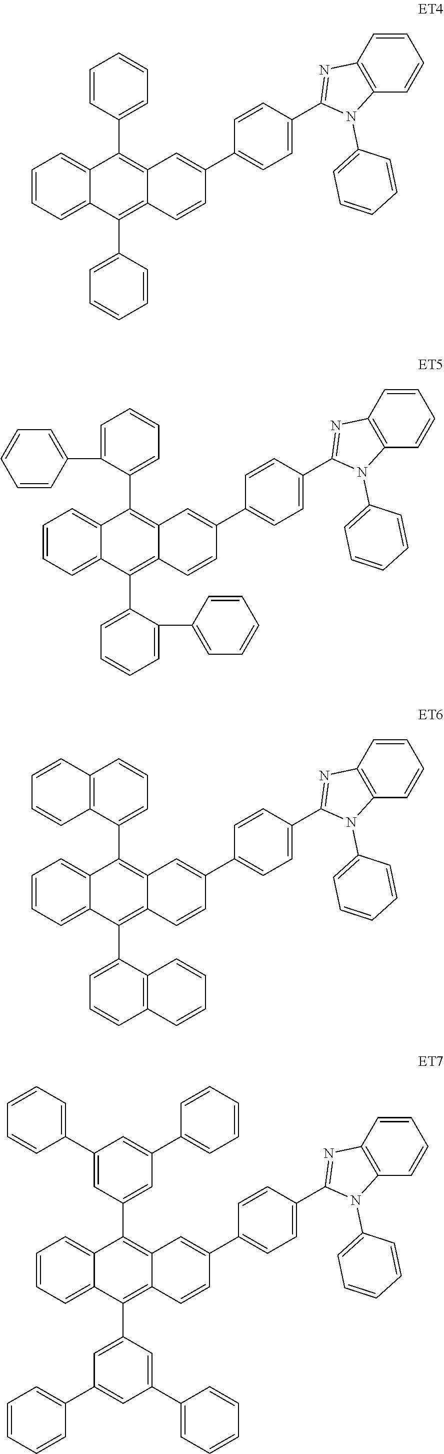 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

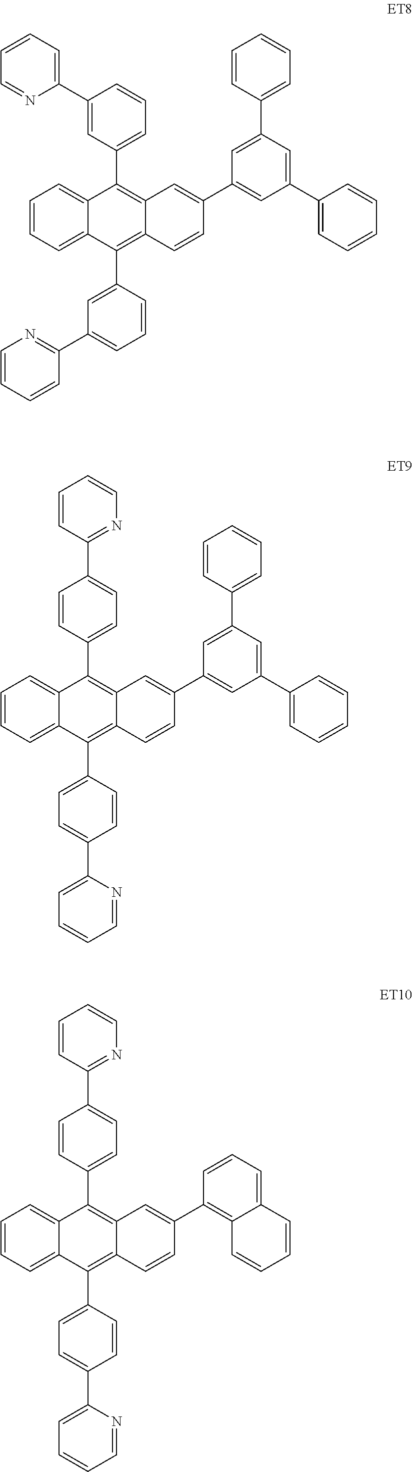 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

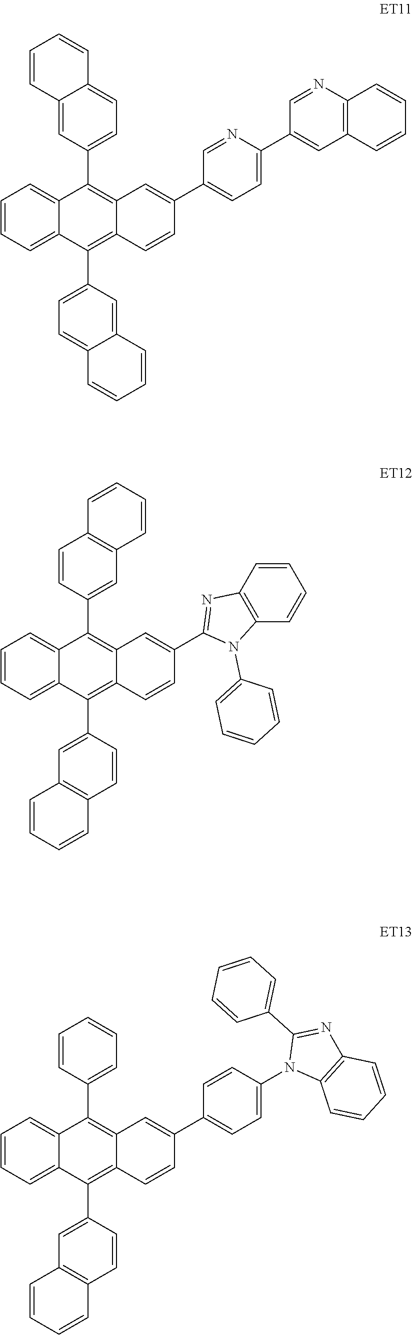 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

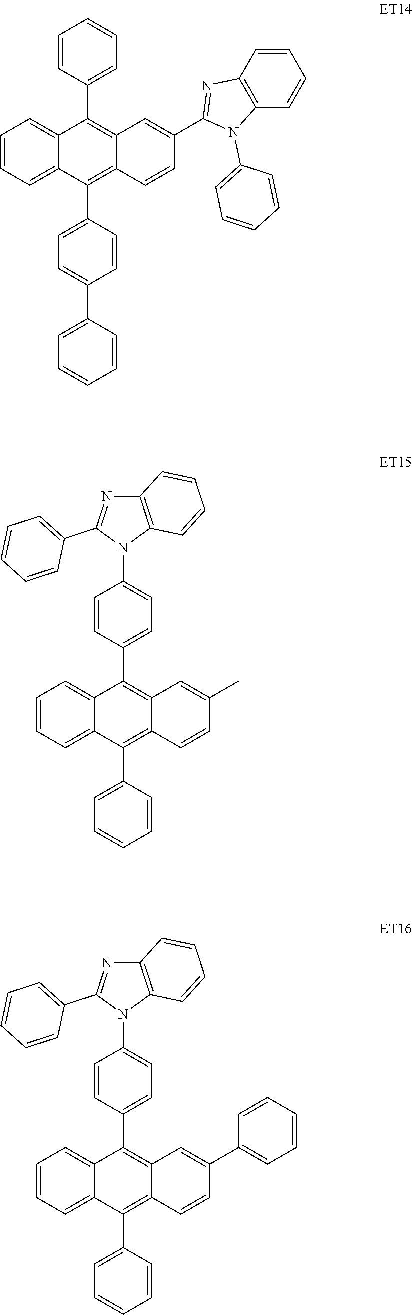 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

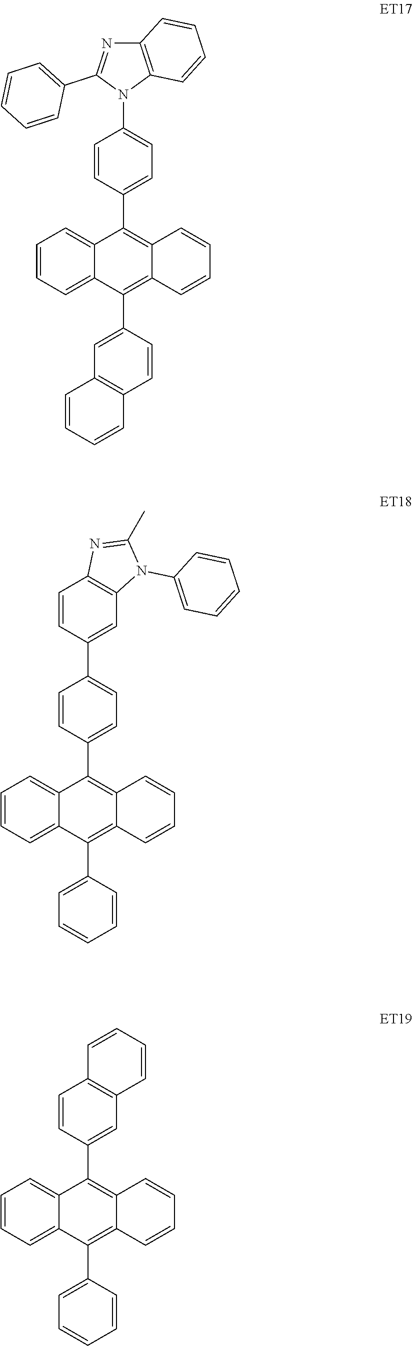

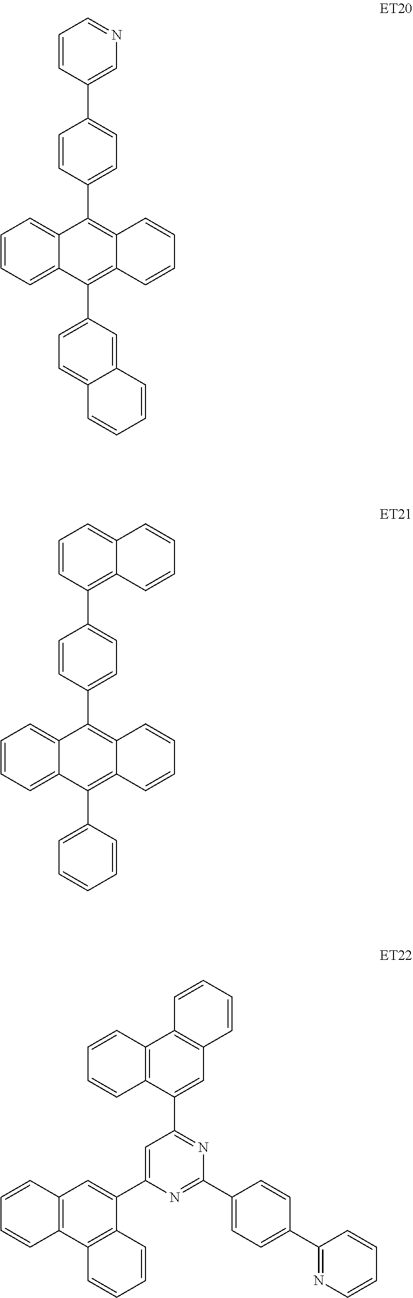

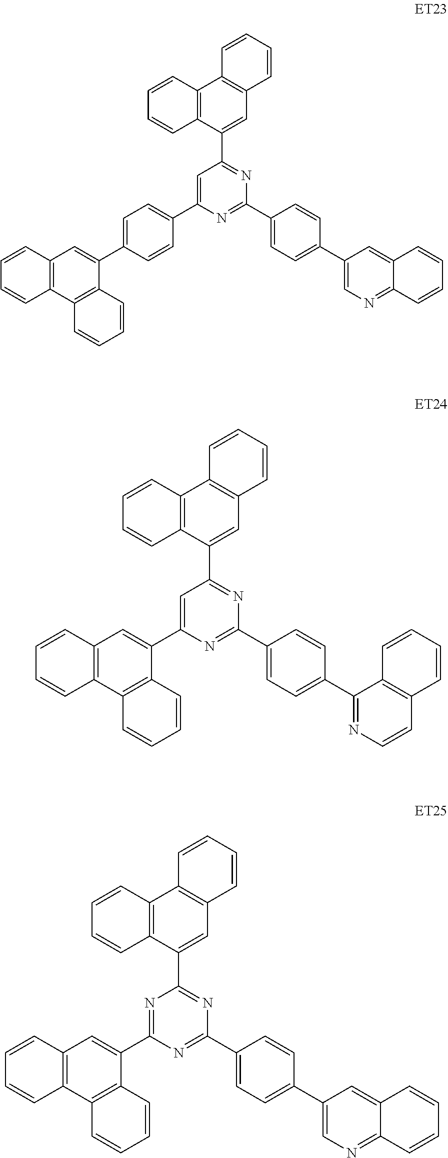 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

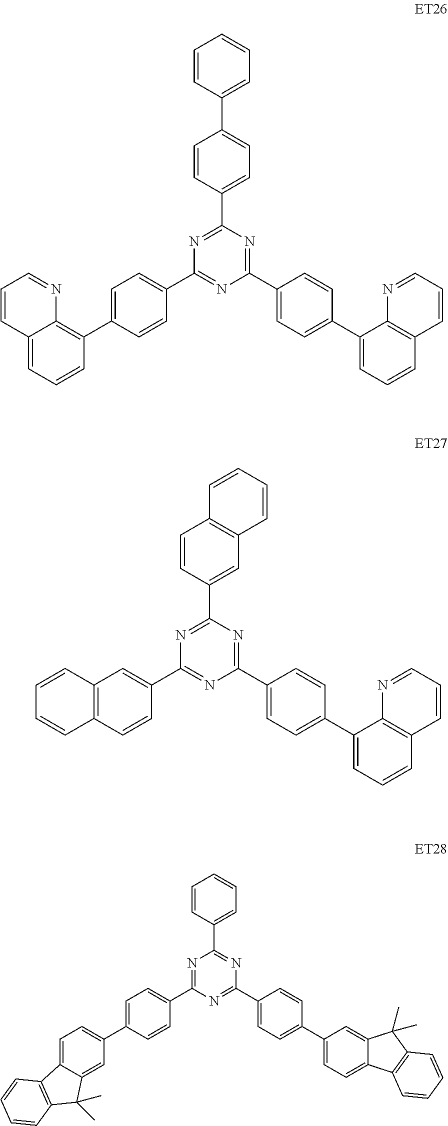 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

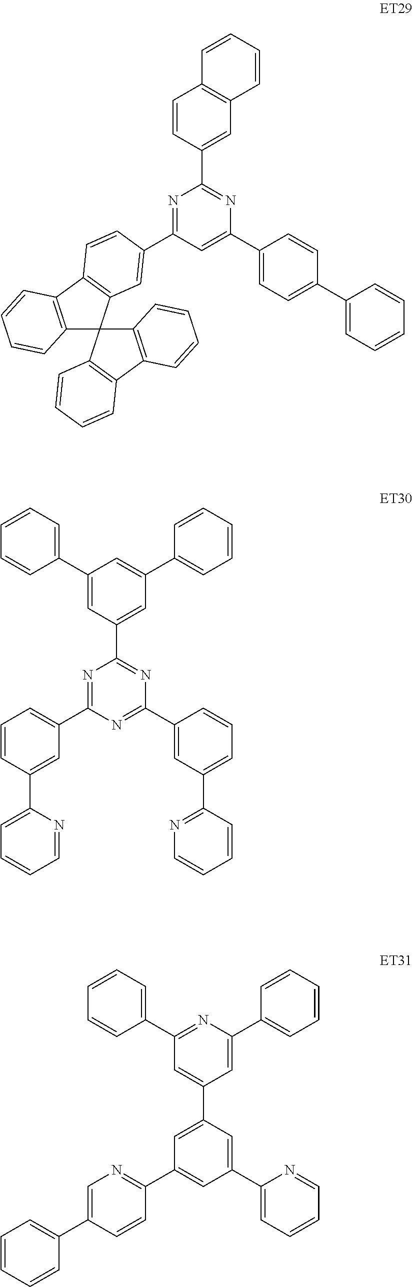 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1]

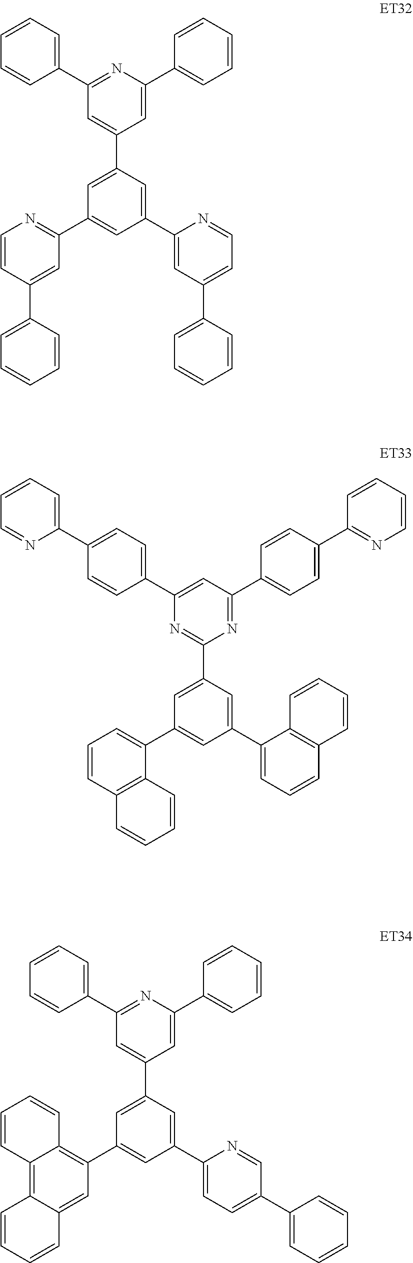 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

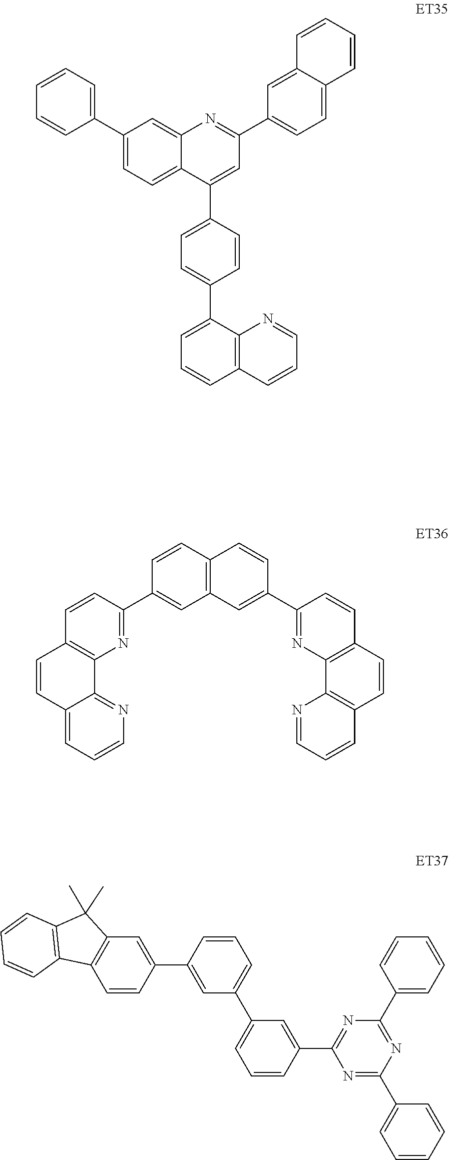 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21]

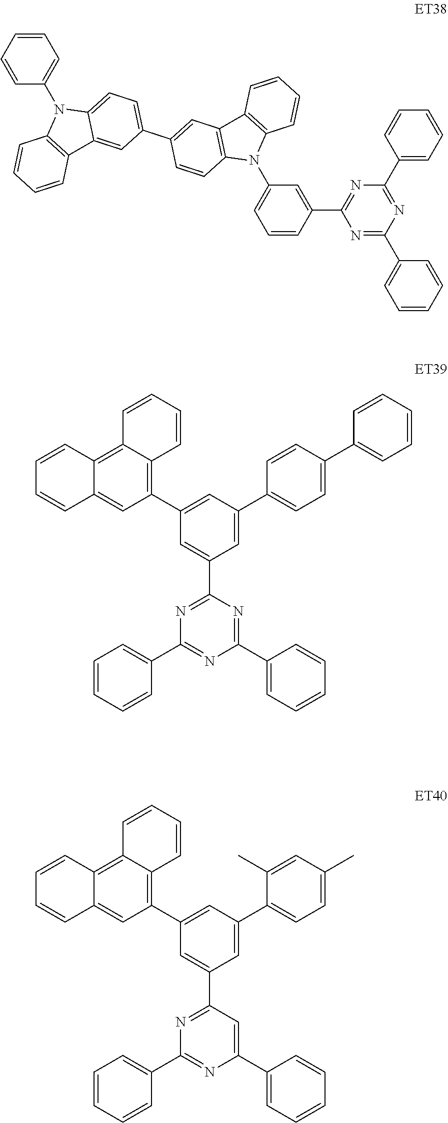 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

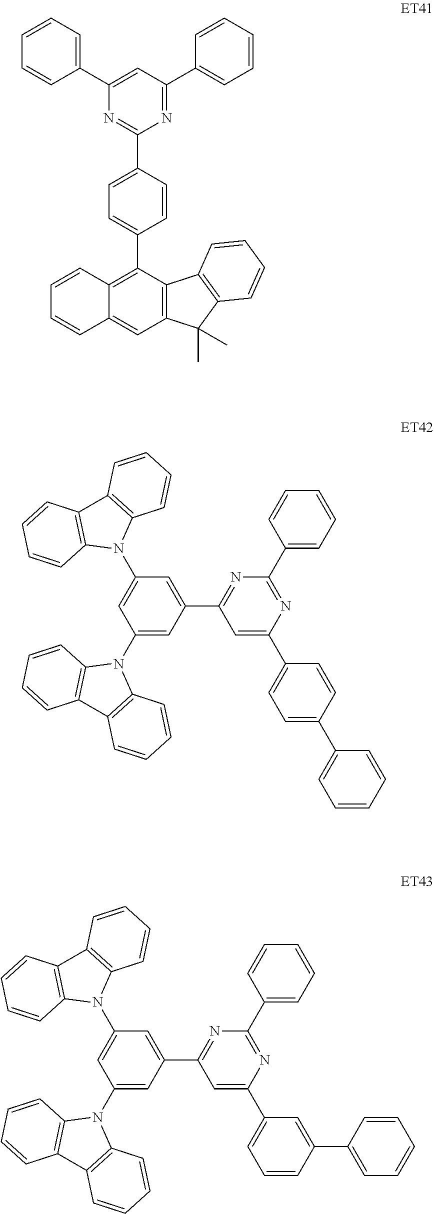 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

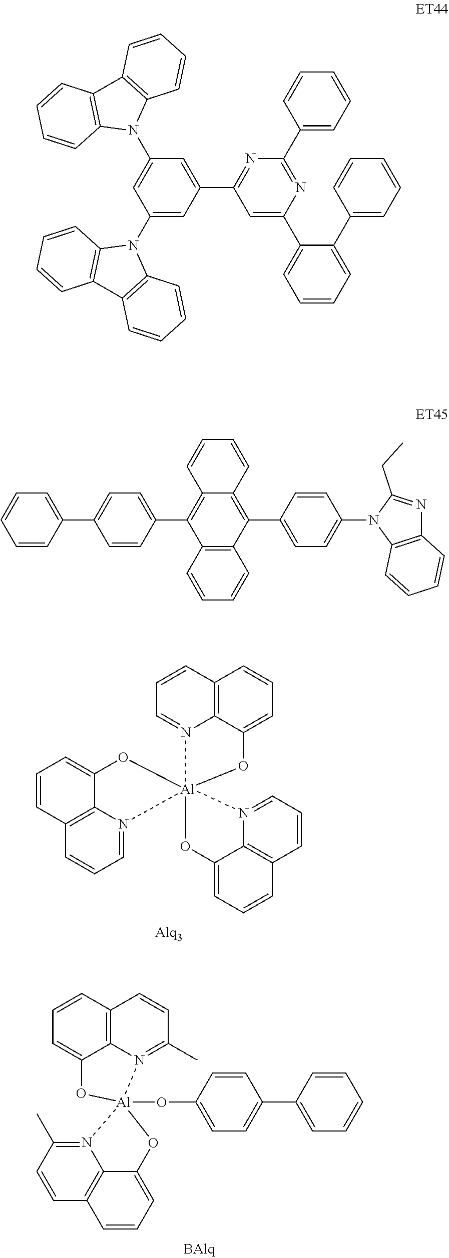

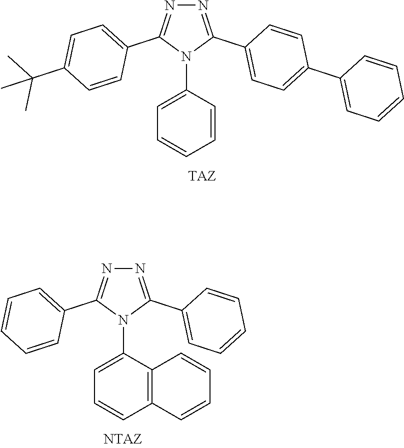 [SMILES: CC(C)(C)c1ccc(-c2nnc(-c3ccc(-c4ccccc4)cc3)n2-c2ccccc2)cc1 ; c1ccc(-c2nnc(-c3ccccc3)n2-c2cccc3ccccc23)cc1]

A thickness of the electron transport region may be in a range of about 100 Å to about 5,000 Å, for example, about 160 Å to about 4,000 Å. When the electron transport region includes the buffer layer, the hole blocking layer, the electron control layer, the electron transport layer, or any combination thereof, a thickness of the buffer layer, the hole blocking layer, and/or the electron control layer may each be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thicknesses of the buffer layer, the hole blocking layer, the electron control layer, the electron transport layer, and/or the electron injection layer are within the respective ranges above, satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth-metal complex, or any combination thereof. A metal ion of the alkali metal complex may be a Li ion, a Na ion, a K ion, a Rb ion, or a Cs ion, and a metal ion of the alkaline earth-metal complex may be a Be ion, a Mg ion, a Ca ion, a Sr ion, or a Ba ion. A ligand coordinated with the metal ion of the alkali metal complex or the alkaline earth-metal complex may be a hydroxy quinoline, a hydroxy isoquinoline, a hydroxy benzoquinoline, a hydroxy acridine, a hydroxy phenanthridine, a hydroxy phenyloxazole, a hydroxy phenylthiazole, a hydroxy diphenyloxadiazole, a hydroxy diphenylthiadiazole, a hydroxy phenylpyridine, a hydroxy phenylbenzimidazole, a hydroxy phenylbenzothiazole, a bipyridine, a phenanthroline, a cyclopentadiene, or any combination thereof.

For example, the metal-containing material may include a Li complex. The Li complex may include, for example, Compound ET-D1 (LiQ) or ET-D2:

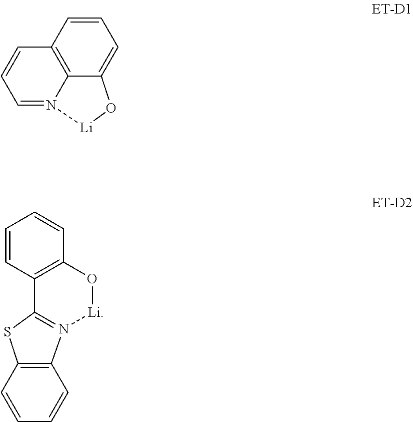

The electron transport region may include an electron injection layer that facilitates electron injection from the second electrode 150. The electron injection layer may directly contact the second electrode 150.

The electron injection layer may have: i) a single-layered structure consisting of a single layer consisting of a single material, ii) a single-layered structure consisting of a single layer including (e.g., consisting of) a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides and/or halides (for example, fluorides, chlorides, bromides, and/or iodides), and/or telluride of the alkali metal, the alkaline earth metal, and the rare earth metal, or any combination thereof.

The alkali metal-containing compound may be alkali metal oxides (such as Li₂O, Cs₂O, and/or K₂O), alkali metal halides (such as LiF, NaF, CsF, KF, LiI, NaI, CsI, and/or KI), or any combination thereof. The alkali earth metal-containing compound may be an alkali earth metal oxides, such as BaO, SrO, CaO, BaₓSr₁₋ₓO (where x is a real number satisfying 0<x<1), and/or BaₓCa₁₋ₓO (where x is a real number satisfying 0<x<1). The rare earth metal-containing compound may be YbF₃, ScF₃, Sc₂O₃, Y₂O₃, Ce₂O₃, GdF₃, TbF₃, YbI₃, ScI₃, TbI₃, or any combination thereof. In an embodiment, the rare earth metal-containing compound may be lanthanide metal telluride. Non-limiting examples of the lanthanide metal telluride are LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and/or the like.

The alkali metal complex, the alkaline earth-metal complex, and the rare earth metal complex may include i) one of ions of the alkali metal, the alkaline earth metal, and the rare earth metal and ii), as a ligand linked to the metal ion, for example, a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxydiphenyloxadiazole, a hydroxydiphenylthiadiazole, a hydroxyphenylpyridine, a hydroxyphenyl benzimidazole, a hydroxyphenyl benzothiazole, a bipyridine, a phenanthroline, a cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof, or may further include an organic material (for example, a compound represented by Formula 601).

In an embodiment, the electron injection layer includes (e.g., consists of) i) an alkali metal-containing compound (for example, an alkali metal halide), or ii) a) an alkali metal-containing compound (for example, an alkali metal halide); and b) an alkali metal, an alkali earth metal, a rare earth metal, or any combination thereof. For example, the electron injection layer may be a KI:Yb co-deposited layer, a RbI:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be uniformly or non-uniformly dispersed in a matrix including the organic material.

A thickness of the electron injection layer may be in a range of about 1 Å to about 100 Å, for example, about 3 Å to about 90 Å. When the thickness of the electron injection layer is within the ranges above, satisfactory electron injection characteristics may be obtained without a substantial increase in driving voltage.

### Second Electrode 150

The second electrode 150 is located on the interlayer 130 having such a structure. The second electrode 150 may be a cathode, which is an electron injection electrode, and as a material for forming the second electrode 150, a metal, an alloy, an electrically conductive compound, or any combination thereof, each having a low work function, may be utilized.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including two or more layers.

### Capping Layer

A first capping layer may be located outside the first electrode (e.g., on the side opposite to the second electrode) 110, and/or a second capping layer may be located outside the second electrode (e.g., on the side opposite to the first electrode) 150. In one embodiment, the light-emitting device 10 may have a structure in which the first capping layer, the first electrode 110, the interlayer 130, and the second electrode 150 are sequentially stacked in this stated order, a structure in which the first electrode 110, the interlayer 130, the second electrode 150, and the second capping layer are sequentially stacked in this stated order, or a structure in which the first capping layer, the first electrode 110, the interlayer 130, the second electrode 150, and the second capping layer are sequentially stacked in this stated order.

Light generated in an emission layer of the interlayer 130 of the light-emitting device 10 may be directed or extracted toward the outside through the first electrode 110 and the first capping layer, each of which may be a semi-transmissive material (e.g., a semi-transmissive electrode or layer) or a transmissive material (e.g., a transmissive electrode or layer), or light generated in an emission layer of the interlayer 130 of the light-emitting device 10 may be directed or extracted toward the outside through the second electrode 150 and the second capping layer, each of which may be a semi-transmissive material (e.g., a semi-transmissive electrode or layer) or a transmissive material (e.g., a transmissive electrode or layer).

The first capping layer and the second capping layer may increase external luminescence efficiency according to the principle of constructive interference. Accordingly, the light-emitting device 10 may have improved light emission efficiency, so that the light-emitting device 10 may have improved luminescence efficiency.

The first capping layer and the second capping layer may each include a material having a refractive index of 1.6 or more (at 589 nm).

The first capping layer and the second capping layer may each independently be an organic capping layer including an organic material, an inorganic capping layer including an inorganic material, or a composite capping layer including an organic material and an inorganic material.

At least one selected from the first capping layer and the second capping layer may each independently include a carbocyclic compound, a heterocyclic compound, an amine group-containing compound, a porphyrine derivative, a phthalocyanine derivative, a naphthalocyanine derivative, an alkali metal complex, an alkaline earth-metal complex, or a combination thereof. The carbocyclic compound, the heterocyclic compound, and the amine group-containing compound may be optionally substituted with a substituent containing O, N, S, Se, Si, F, Cl, Br, I, or any combination thereof. In an embodiment, at least one of the first capping layer or the second capping layer may (e.g., each independently) include an amine group-containing compound.

For example, at least one of the first capping layer or the second capping layer may (e.g., each independently) include a compound represented by Formula 201, a compound represented by Formula 202, or any combination thereof.

In one or more embodiments, at least one of the first capping layer or the second capping layer may (e.g., each independently) include one of Compounds HT28 to HT33, one of Compounds CP1 to CP6, β-NPB, or any combination thereof:

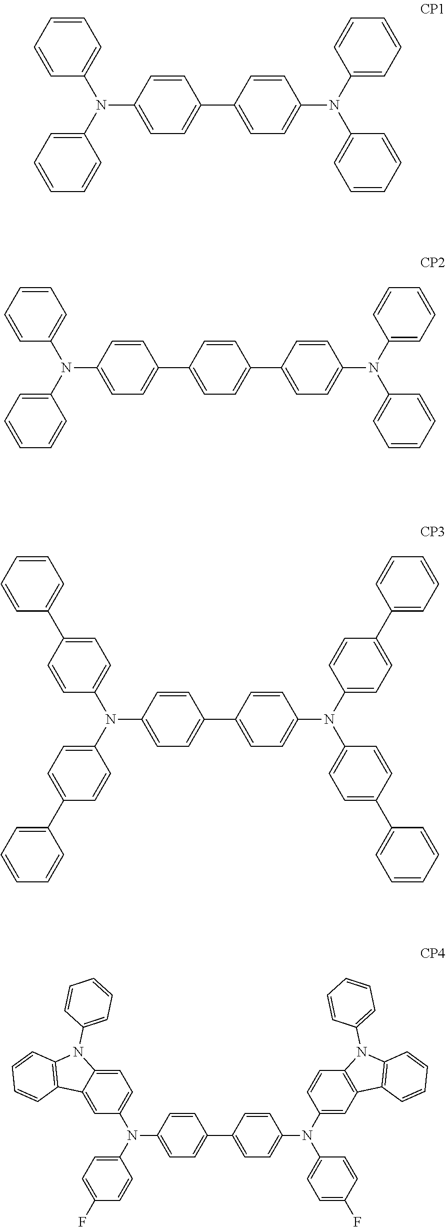 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

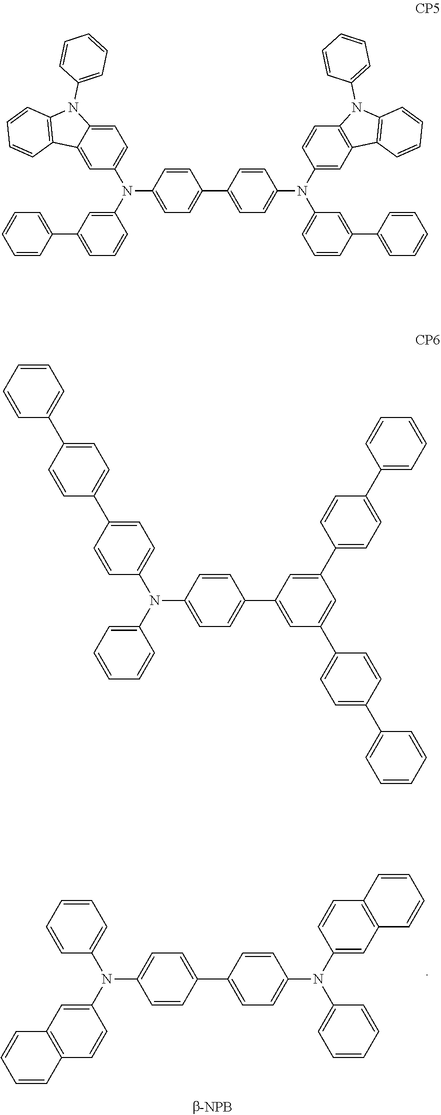 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5cc(-c6ccc(-c7ccccc7)cc6)cc(-c6ccc(-c7ccccc7)cc6)c5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Electronic Apparatus

The light-emitting device may be included in various suitable apparatuses. For example, an electronic apparatus including the light-emitting device may be a light-emitting apparatus, an authentication apparatus, and/or the like.

The electronic apparatus (for example, a light-emitting apparatus) may further include, in addition to the light-emitting device, i) a color filter, ii) a color conversion layer, or iii) a color filter and a color conversion layer. The color filter and/or the color conversion layer may be placed in at least one traveling direction of light emitted from the light-emitting device. For example, light emitted from the light-emitting device may be blue light or white light. The light-emitting device may be the same as described above. In an embodiment, the color conversion layer may include a quantum dot. The quantum dot may be, for example, the quantum dot described above.

The electronic apparatus may include a first substrate. The first substrate may include a plurality of subpixel areas, the color filter may include a plurality of color filter areas respectively corresponding to the plurality of subpixel areas, and the color conversion layer may include a plurality of color conversion areas respectively corresponding to the plurality of subpixel areas.

A pixel-defining film may be placed between the plurality of subpixel areas to define each of the subpixel areas.

The color filter may further include light-blocking patterns placed between the plurality of color filter areas, and the color conversion layer may further include light-blocking patterns placed between the plurality of color conversion areas.

The plurality of color filter areas (or the plurality of color conversion areas) may include: a first area emitting first color light; a second area emitting second color light; and/or a third area emitting third color light, and the first color light, the second color light, and/or the third color light may have different maximum luminescence wavelengths from one another. For example, the first color light may be red light, the second color light may be green light, and the third color light may be blue light. For example, the plurality of color filter areas (or, the plurality of color conversion areas) may include quantum dots. In one embodiment, the first area may include a red quantum dot, the second area may include a green quantum dot, and the third area may not include a quantum dot. The quantum dot may be the same as described in the present specification. Each of the first region, the second region, and/or the third region may further include a scatterer.

For example, the light-emitting device may emit a first light, the first area may absorb the first light to emit first first-color light, the second area may absorb the first light to emit second first-color light, and the third area may absorb the first light to emit third first-color light. Here, the first first-color light, the second first-color light, and the third first-color light may have different maximum luminescence wavelengths from one another. In one embodiment, the first light may be blue light, the first first-color light may be red light, the second first-color light may be green light, and the third first-color light may be blue light. In one embodiment, the first light may be white light, the first first-color light may be red light, the second first-color light may be green light, and the third first-color light may be blue light.

The electronic apparatus may include the light-emitting device and a thin-film transistor, wherein the thin-film transistor includes a source electrode and a drain electrode, and the first electrode of the light-emitting device is electrically connected to the source electrode or the drain electrode of the thin-film transistor.

The thin-film transistor may further include an active layer, a gate electrode, a gate insulation film, and/or the like.

The active layer may include crystalline silicon, amorphous silicon, an organic semiconductor, an oxide semiconductor, and/or the like, but embodiments of the present disclosure are not limited thereto.

The electronic apparatus may further include a sealing portion for sealing the light-emitting device. The sealing portion may be placed between the light-emitting device and the color filter and/or between the light-emitting device and the color conversion layer. The sealing portion allows light from the light-emitting device to be extracted to the outside, while concurrently (or simultaneously) preventing or substantially preventing external air and moisture from penetrating into the light-emitting device. The sealing portion may be a sealing substrate including a transparent glass substrate or a plastic substrate. The sealing portion may be a thin-film encapsulation layer including one or more organic layers and/or one or more inorganic layers. When the sealing portion is a thin-film encapsulation layer, the electronic apparatus may be flexible.

On the sealing portion, various suitable functional layers other than the color filter and/or the color conversion layer may be additionally placed depending on the usage of the electronic apparatus. Non-limiting examples of the functional layers are a touchscreen layer, a polarization layer, and/or the like. The touchscreen layer may be a resistive touchscreen layer, a capacitive touchscreen layer, or an infrared touchscreen layer. The authentication apparatus may be, for example, a biometric authentication apparatus for authenticating an individual by utilizing biometric information of a biometric body (for example, a fingertip, a pupil, and/or the like).

The authentication apparatus may further include, in addition to the light-emitting device, a biometric information collector.

The electronic apparatus may be applied to various suitable displays, light sources, lighting apparatuses, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram (ECG) displays, ultrasonic diagnostic devices, and/or endoscope displays), fish finders, various measuring instruments, meters (for example, meters for a vehicle, an aircraft, and/or a vessel), projectors, and/or the like.

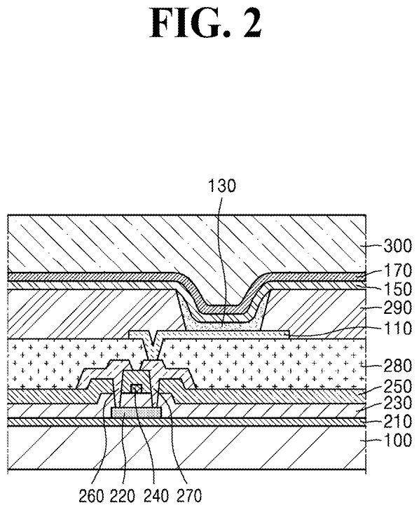

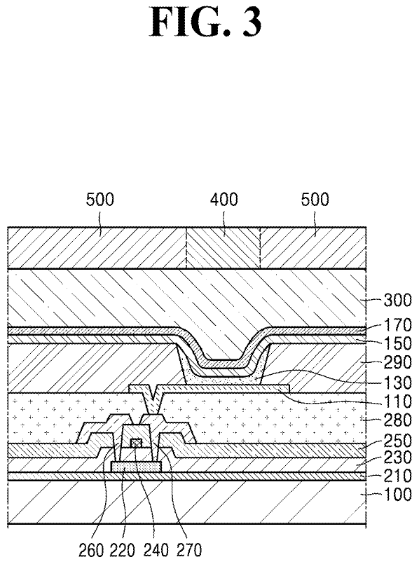

### Description of FIGS. 2 and 3

FIG. 2 is a cross-sectional view showing a light-emitting apparatus according to an embodiment of the present disclosure.

The light-emitting apparatus of FIG. 2 includes a substrate 100, a thin-film transistor (TFT), a light-emitting device, and an encapsulation part 300 that seals the light-emitting device.

The substrate 100 may be a flexible substrate, a glass substrate, or a metal substrate. A buffer layer 210 may be placed on the substrate 100. The buffer layer 210 may prevent or reduce penetration of impurities through the substrate 100, and may serve to provide a flat surface on the substrate 100.

A TFT may be placed on the buffer layer 210. The TFT may include an active layer 220, a gate electrode 240, a source electrode 260, and a drain electrode 270.

The active layer 220 may include an inorganic semiconductor, such as silicon and/or polysilicon, an organic semiconductor, and/or an oxide semiconductor, and may include a source region, a drain region and a channel region.

A gate insulating layer 230 for insulating the active layer 220 and the gate electrode 240 may be placed on the active layer 220, and the gate electrode 240 may be placed on the gate insulating layer 230.

An interlayer insulating layer 250 may be placed on the gate electrode 240. The interlayer insulating layer 250 may be placed between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be placed on the interlayer insulating layer 250. The interlayer insulating layer 250 and the gate insulating layer 230 may be formed to expose a source region and a drain region of the active layer 220, and the source electrode 260 and the drain electrode 270 may be in contact with the exposed portions of the source region and the drain region of the active layer 220.

The TFT may be electrically connected to the light-emitting device to drive the light-emitting device, and may be covered by a passivation layer 280 for protection.

The passivation layer 280 may include an inorganic insulating film, an organic insulating film, or a combination thereof. A light-emitting device may be provided on the passivation layer 280. The light-emitting device includes a first electrode 110, an interlayer 130, and a second electrode 150.

The first electrode 110 may be placed on the passivation layer 280. The passivation layer 280 may be placed to expose a certain portion of the drain electrode 270 without completely covering the drain electrode 270, and the first electrode 110 may be connected to the exposed portion of the drain electrode 270.

A pixel defining layer 290 including an insulating material may be placed on the first electrode 110. The pixel defining layer 290 may expose a certain region of the first electrode 110, and an interlayer 130 may be formed in the exposed region of the first electrode 110. The pixel defining layer 290 may be a polyimide or polyacryl-based organic film. In one more embodiments, one or more layers of the interlayer 130 may be extended to an upper portion of the pixel defining layer 290 to be placed in the form of a common layer.

The second electrode 150 may be placed on the interlayer 130, and a capping layer 170 may be additionally formed on the second electrode 150. The capping layer 170 may be formed to cover the second electrode 150.

An encapsulation part 300 may be placed on the capping layer 170. The encapsulation part 300 may be placed on the light-emitting device to protect the light-emitting device from moisture or oxygen. The encapsulation part 300 may include an inorganic film (such as silicon nitride (SiNx), silicon oxide (SiOx), indium tin oxide, indium zinc oxide, or any combination thereof), an organic film (such as polyethylene terephthalate, polyethylene naphthalate, polycarbonate, polyimide, polyethylene sulfonate, polyoxymethylene, polyarylate, hexamethyldisiloxane, an acryl-based resin (for example, polymethyl methacrylate, polyacrylic acid, and/or the like), an epoxy-based resion (for example, aliphatic glycidyl ether (AGE) and/or the like), or a combination thereof), or a combination of an inorganic film and an organic film.

FIG. 3 is a cross-sectional view showing a light-emitting apparatus according to another embodiment of the present disclosure.

The light-emitting apparatus of FIG. 3 is the same as the light-emitting apparatus of FIG. 2, except that a light-blocking pattern 500 and a functional area 400 are additionally placed on the encapsulation part 300 in the light-emitting apparatus of FIG. 3. The functional area 400 may be i) a color filter area, ii) a color conversion area, or iii) a combination of a color filter area and a color conversion area. In an embodiment, the light-emitting device included in the light-emitting apparatus of FIG. 3 may be a tandem light-emitting device.

### Preparation Method

Layers constituting the hole transport region, the emission layer, and layers constituting the electron transport region may be formed in a certain region by utilizing one or more suitable methods selected from vacuum deposition, spin coating, casting, Langmuir-Blodgett (LB) deposition, ink-jet printing, laser-printing, and laser-induced thermal imaging.

When layers constituting the hole transport region, the emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec by taking into account a material to be included in a layer to be formed and the structure of a layer to be formed.

### Definitions of Terms

The term “C₃-C₆₀ carbocyclic group” as used herein refers to a cyclic group including only carbon atoms as ring-forming atoms and having 3 to 60 carbon atoms, and the term “C₁-C₆₀ heterocyclic group” as used herein refers to a cyclic group further including a heteroatom, other than carbon atoms, as a ring-forming atom and having 1 to 60 carbon atoms. The C₃-C₆₀ carbocyclic group and the C₁-C₆₀ heterocyclic group may each independently be a monocyclic group consisting of one ring or a polycyclic group having two or more rings condensed with each other. For example, the number of the ring-forming atoms of the C₁-C₆₀ heterocyclic group may be 3 to 61.

The term “cyclic group” as used herein includes both the C₃-C₆₀ carbocyclic group and the C₁-C₆₀ heterocyclic group.

The term “T₁ electron-rich C₃-C₆₀ cyclic group” as used herein refers to a carbocyclic group and having 3 to 60 carbon atoms, or a heterocyclic group not including *—N═*′ as a ring-forming moiety and having 3 to 60 carbon atoms. The term “T₁ electron-deficient nitrogen-containing C₁-C₆₀ cyclic group” as used herein refers to a heterocyclic group including *—N═*′ as a ring-forming moiety and having 1 to 60 carbon atoms.

For example, the C₃-C₆₀ carbocyclic group may be i) a T₁ group or ii) a condensed cyclic group in which two or more T₁ groups are condensed with each other (for example, a cyclopentadiene group, an adamantane group, a norbornane group, a benzene group, a pentalene group, a naphthalene group, an azulene group, an indacene group, an acenaphthylene group, a phenal ene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) a T₂ group, ii) a condensed cyclic group in which two or more T₂ groups are condensed with each other, or iii) a condensed cyclic group in which at least one T₂ group and at least one T₁ group are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),

the π electron-rich C₃-C₆₀ cyclic group may be i) a T1 group, ii) a condensed cyclic group in which two or more T1 groups are condensed with each other, iii) a T3 group, iv) a condensed cyclic group in which two or more T3 groups are condensed with each other, or v) a condensed cyclic group in which at least one T3 group and at least one T1 group are condensed with each other (for example, the C₃-C₆₀ carbocyclic group, a 1H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like),

the π electron-deficient nitrogen-containing C₁-C₆₀ cyclic group may be i) a T4 group, ii) a condensed cyclic group in which two or more T4 groups are condensed with each other, iii) a condensed cyclic group in which at least one T4 group and at least one T1 group are condensed with each other, iv) a condensed cyclic group in which at least one T4 group and at least one T3 group are condensed with each other, or v) a condensed cyclic group in which at least one T4 group, at least one T1 group, and at least one T3 group are condensed with each other (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),

the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane group (or, a bicyclo[2.2.1]heptane group), a norbornene group, a bicyclo[1.1.1]pentane group, a bicyclo[2.1.1]hexane group, a bicyclo[2.2.2]octane group, or a benzene group,

the T2 group may be a furan group, a thiophene group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, a dihydropyridazine group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group,

the T3 group may be a furan group, a thiophene group, a 1H-pyrrole group, a silole group, or a borole group, and

the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group”, “C₃-C₆₀ carbocyclic group”, “C₁-C₆₀ heterocyclic group”, “π electron-rich C₃-C₆₀ cyclic group” or “π electron-deficient nitrogen-containing C₁-C₆₀ cyclic group” as used herein each refers to, depending on the structure of the formula for the terms as used herein, a group condensed to any cyclic group, or a monovalent or multivalent group (for example, a divalent group, a trivalent group, a tetravalent group, and/or the like). For example, the term “benzene group” may be a benzo group, a phenyl group, a phenylene group, and/or the like, which will be easily understood by those skilled in the art according to the structure of the formula including the “benzene group”.

Non-limiting examples of a monovalent C₃-C₆₀ carbocyclic group and a monovalent C₁-C₆₀ heterocyclic group are a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and non-limiting examples of a divalent C₃-C₆₀ carbocyclic group and a divalent C₁-C₆₀ heterocyclic group are a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group having 1 to 60 carbon atoms, and examples thereof are a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term

“C₁-C₆₀ alkylene group” as used herein refers to a divalent group having the same structure as the C₁-C₆₀ alkyl group.

The term “C₂-C₆₀ alkenyl group” as used herein refers to a monovalent hydrocarbon group having at least one carbon-carbon double bond in the middle or at a terminal end (e.g., the terminus) of a C₂-C₆₀ alkyl group, and non-limiting examples thereof are an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a monovalent hydrocarbon group having at least one carbon-carbon triple bond in the middle or at a terminal end (e.g., the terminus) of a C₂-C₆₀ alkyl group, and non-limiting examples thereof are an ethynyl group and a propynyl group. The term “C₂-C₆₀ alkynylene group” as used herein refers to a divalent group having the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and non-limiting examples thereof are a methoxy group, an ethoxy group, and an isopropyloxy group.

The term “C₃-C₁₀ cycloalkyl group” as used herein refers to a monovalent saturated hydrocarbon cyclic group having 3 to 10 carbon atoms, and non-limiting examples thereof are a cyclopropyl group, a cyclobutyl group, a cyclopentyl group, a cyclohexyl group, a cycloheptyl group, a cycloctyl group, an adamantanyl group, a norbornanyl group (or a bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent cyclic group further including at least one heteroatom, other than carbon atoms, as a ring-forming atom and having 1 to 10 carbon atoms, and non-limiting examples thereof are a 1,2,3,4-oxatriazolidinyl group, a tetrahydrofuranyl group, and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent monocyclic group that has 3 to 10 carbon atoms and at least one carbon-carbon double bond in the ring thereof and no aromaticity, and non-limiting examples thereof are a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent cyclic group further including at least one heteroatom, other than carbon atoms, as a ring-forming atom, having 1 to 10 carbon atoms, and including at least one double bond in its ring. Non-limiting examples of the C₁-C₁₀ heterocycloalkenyl group are a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Non-limiting examples of the C₆-C₆₀ aryl group are a phenyl group, a pentalenyl group, a naphthyl group, an azulenyl group, an indacenyl group, an acenaphthyl group, a phenal enyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, a fluorenyl group and an ovalenyl group. When the C₆-C₆₀ aryl group and the C₆-C₆₀ arylene group each include two or more rings, the two or more rings may be condensed with each other.

The term “C₁-C₆₀ heteroaryl group” as used herein refers to a monovalent group having an aromatic system that further includes at least one heteroatom, other than carbon atoms, as a ring-forming atom and has 1 to 60 carbon atoms. The term “C₁-C₆₀ heteroarylene group” as used herein refers to a divalent group having an aromatic system that further includes at least one heteroatom, other than carbon atoms, as a ring-forming atom and has 1 to 60 carbon atoms. Non-limiting examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a carbazolyl group, a dibenzofuranyl group, a dibenzothiofuranyl group, and a naphthyridinyl group. When the C₁-C₆₀ heteroaryl group and the C₁-C₆₀ heteroarylene group each include two or more rings, the two or more rings may be condensed with each other.

The term “monovalent non-aromatic condensed polycyclic group” as used herein refers to a monovalent group having two or more rings condensed with each other, only carbon atoms (for example, having 8 to 60 carbon atoms) as ring-forming atoms, and no aromaticity in its entire molecular structure (e.g., the entire molecular structure is not aromatic). Non-limiting examples of the monovalent non-aromatic condensed polycyclic group are an indenyl group, a fluorenyl group, a spiro-bifluorenyl group, a benzofluorenyl group, an indenophenanthrenyl group, an adamantyl group, and an indenoanthracenyl group. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group (for example, having 1 to 60 carbon atoms) having two or more rings condensed with each other, further including at least one heteroatom, other than carbon atoms, as a ring-forming atom, and having non-aromaticity in its entire molecular structure (e.g., the entire molecular structure is not aromatic). Non-limiting examples of the monovalent non-aromatic condensed heteropolycyclic group are a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, an azaadamantyl group, and a benzothienodibenzothiophenyl group. The term “divalent non-aromatic condensed heteropolycyclic group” as used herein refers to a divalent group having the same structure as the monovalent non-aromatic condensed heteropolycyclic group.

The term “C₆-C₆₀ aryloxy group” as used herein refers to a monovalent group represented by —OA₁₀₂ (wherein A₁₀₂ is the C₆-C₆₀ aryl group), and the term “C₆-C₆₀ arylthio group” as used herein refers to a monovalent group represented by —SA₁₀₃ (wherein A₁₀₃ is the C₆-C₆₀ aryl group).

The term “C₇-C₆₀ arylalkyl group” as used herein refers to a monovalent group represented by -A₁₀₄A₁₀₅ (wherein A₁₀₄ is a C₁-C₅₄ alkylene group, and A₁₀₅ is a C₆-C₅₉ aryl group), and the term “C₂-C₆₀ heteroarylalkyl group” as used herein refers to a monovalent group represented by -A₁₀₆A₁₀₇ (wherein A₁₀₆ is a C₁-C₅₉ alkylene group, and A₁₀₇ is a C₁-C₅₉ heteroaryl group).

“R₁₀ₐ” as used herein may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —C₁, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Throughout the current description, the expression “at least one R₁₀ₐ” refers to at least one of the groups described above in connection with R₁₀ₐ.

In the present specification, Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

The term “heteroarom” as used herein refers to any atom except a carbon atom. Non-limiting examples of the heteroatom are O, S, N, P, Si, B, Ge, Se, or any combination thereof.

The term “Ph” as used herein refers to a phenyl group, the term “Me” as used herein refers to a methyl group, the term “Et” as used herein refers to an ethyl group, the term “ter-Bu” or “But” as used herein refers to a tert-butyl group, and the term “OMe” as used herein refers to a methoxy group.

The term “biphenyl group” as used herein refers to “a phenyl group substituted with a phenyl group”. In other words, the “biphenyl group” is a substituted phenyl group having a C₆-C₆₀ aryl group as a substituent.

The term “terphenyl group” as used herein refers to “a phenyl group substituted with a biphenyl group”. In other words, the “terphenyl group” is a substituted phenyl group having, as a substituent, a C₆-C₆₀ aryl group substituted with a C₆-C₆₀ aryl group.

* and *′ as used herein, unless defined otherwise, each refer to a binding site to a neighboring atom in a corresponding formula.

Hereinafter, a compound according to embodiments and a light-emitting device according to embodiments will be described in more detail with reference to Synthesis Examples and Examples. The wording “B was utilized instead of A” used in describing Synthesis Examples refers to that an identical molar equivalent of B was utilized in place of A.

### EXAMPLES

### Evaluation Example 1: Evaluation of T₁ and S₁ Energy Levels

According to the methods described above, lowest excitation triplet energy levels (T₁) and lowest excitation singlet energy levels (S₁) of the following compounds were evaluated, and results thereof are shown in Tables 1 to 3.

TABLE 1

| First host | T₁ (eV) of first host |
|---|---|
| 1-20 | 2.94 |
| 1-21 | 3.05 |
| 1-23 | 3.01 |
| 1-24 | 2.95 |

TABLE 2

| Second host | T₁ (eV) of second host |
|---|---|
| 2-7 | 3.01 |
| 2-8 | 2.87 |
| 2-11 | 2.96 |
| 2-12 | 2.98 |
| 2-13 | 3.01 |

TABLE 3

| Exciplex | First host | Second host | S₁ (eV) of exciplex |
|---|---|---|---|
| Ex 1 | 1-20 | 2-7 | 2.60 |
| Ex 2 | 1-24 | 2-7 | 2.60 |
| Ex 3 | 1-21 | 2-11 | 2.65 |
| Comparative Ex 1 | 1-23 | 2-11 | 2.93 |
| Comparative Ex 2 | 1-20 | 2-12 | 2.50 |
| Comparative Ex 3 | 1-20 | 2-13 | 2.51 |
| Comparative Ex 4 | 1-20 | 2-8 | 2.67 |

### Evaluation Example 2: Calculation of TCF

Based on the results of Tables 1 to 3, TCF values were calculated, and results thereof are shown in Table 4.

TABLE 4

| First host | Second host | Exciplex | TCF (eV) |
|---|---|---|---|
| 1-20 | 2-7 | Ex 1 | 0.75 |
| 1-24 | 2-7 | Ex 2 | 0.76 |
| 1-21 | 2-11 | Ex 3 | 0.71 |
| 1-23 | 2-11 | Comparative Ex 1 | 0.12 |
| 1-20 | 2-12 | Comparative Ex 2 | 0.93 |
| 1-20 | 2-13 | Comparative Ex 3 | 0.94 |
| 1-20 | 2-8 | Comparative Ex 4 | 0.47 |

### Example 1

As an anode, an ITO substrate was cut to a size of 50 mm×50 mm×0.5 mm, sonicated with isopropyl alcohol and pure water each for 5 minutes, and then cleaned by exposure to ultraviolet rays and ozone for 30 minutes. Then, the ITO substrate was provided to a vacuum deposition apparatus.

m-MTDATA was deposited on the ITO substrate to form a hole injection layer having a thickness of 400 Å, NPB was vacuum-deposited on the hole injection layer to form a hole transport layer having a thickness of 100 Å, and Compounds 1-20, 2-7, and 3-5 were co-deposited on the hole transport layer at a weight ratio of 6:4:1 to form an emission layer having a thickness of 200 Å. Compound ET1 was deposited on the emission layer to form an electron transport layer having a thickness of 300 Å. Al was deposited on the electron transport layer to form a cathode having a thickness of 1,200 Å, thereby completing the manufacture of a light-emitting device.

### Examples 2 and 3 and Comparative Examples 1 to 4

A light-emitting device of each of examples 2 and 3 and comparative examples 1 to 4 was manufactured in the same manner as in Example 1, except that an emission layer was formed by utilizing Compounds shown in Table 5.

### Evaluation Example 3

The efficiency, luminescence wavelength, and lifespan of each of the light-emitting devices manufactured according to Examples 1 to 3 and Comparative Examples 1 to 4 were measured at a current density of 10 mA/cm² by utilizing Keithley SMU 236 and a luminance meter PR650, and results thereof are shown in Table 5. The lifespan is a value (e.g., time duration) measured from an initial 1,000 nit until the luminance reaches 95% of the initial luminance.

TABLE 5

| | Emission layer | | | | |
|---|---|---|---|---|---|
| | | | | Weight ratio | |
| | First | Second | | (first host:second | Lifespan |
| | host | host | Dopant | host:dopant) | (hr) |
| Example 1 | 1-20 | 2-7 | 3-5 | 6:4:1 | 153 |
| Example 2 | 1-24 | 2-7 | 3-5 | 6:4:1 | 190 |
| Example 3 | 1-21 | 2-11 | 3-5 | 6:4:1 | 155 |
| Comparative | 1-23 | 2-11 | 3-5 | 6:4:1 | 24 |
| Example 1 | | | | | |
| Comparative | 1-20 | 2-12 | 3-5 | 6:4:1 | 11 |
| Example 2 | | | | | |
| Comparative | 1-20 | 2-13 | 3-5 | 6:4:1 | 15 |
| Example 3 | | | | | |
| Comparative | 1-20 | 2-8 | 3-5 | 6:4:1 | 33 |
| Example 4 | | | | | |

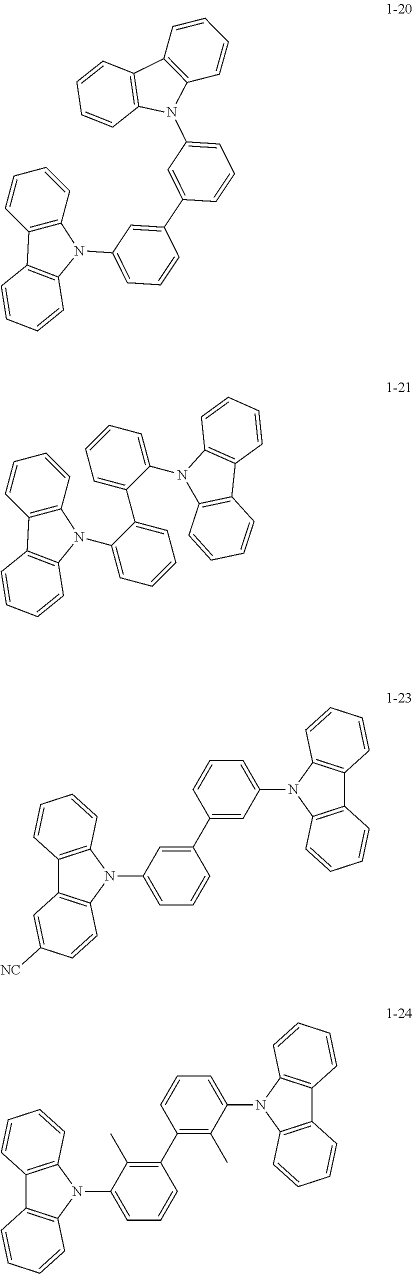 [SMILES: c1cc(-c2cccc(-n3c4ccccc4c4ccccc43)c2)cc(-n2c3ccccc3c3ccccc32)c1 ; c1ccc(-n2c3ccccc3c3ccccc32)c(-c2ccccc2-n2c3ccccc3c3ccccc32)c1 ; N#Cc1ccc2c(c1)c1ccccc1n2-c1cccc(-c2cccc(-n3c4ccccc4c4ccccc43)c2)c1 ; Cc1c(-c2cccc(-n3c4ccccc4c4ccccc43)c2C)cccc1-n1c2ccccc2c2ccccc21]

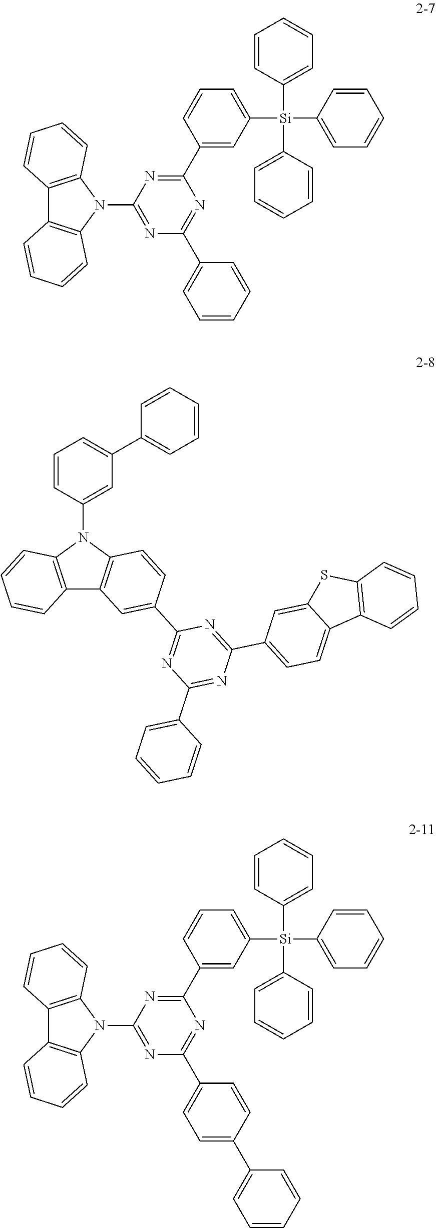 [SMILES: c1ccc(-c2nc(-c3cccc([Si](c4ccccc4)(c4ccccc4)c4ccccc4)c3)nc(-n3c4ccccc4c4ccccc43)n2)cc1 ; c1ccc(-c2cccc(-n3c4ccccc4c4cc(-c5nc(-c6ccccc6)nc(-c6ccc7c(c6)sc6ccccc67)n5)ccc43)c2)cc1 ; c1ccc(-c2ccc(-c3nc(-c4cccc([Si](c5ccccc5)(c5ccccc5)c5ccccc5)c4)nc(-n4c5ccccc5c5ccccc54)n3)cc2)cc1]

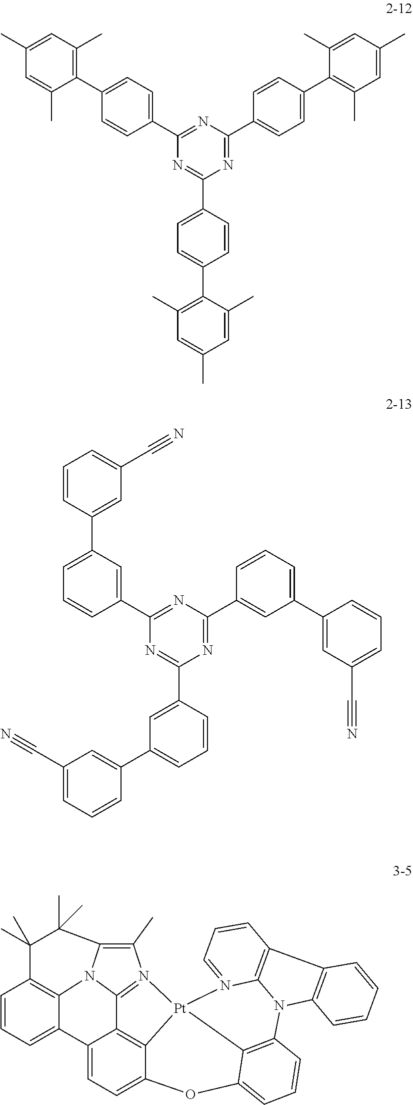

Referring to Table 5, it was confirmed that the light-emitting devices of Examples 1 to 3 had suitable (e.g., excellent) lifespan compared to the light-emitting devices of Comparative Examples 1 to 4.

According to the one or more embodiments, a light-emitting device may have a long lifespan.

The use of “may” when describing embodiments of the present invention refers to “one or more embodiments of the present invention.” Also, the term “exemplary” is intended to refer to an example or illustration. It will be understood that when an element or layer is referred to as being “on”, “connected to”, “coupled to”, or “adjacent to” another element or layer, it can be directly on, connected to, coupled to, or adjacent to the other element or layer, or one or more intervening elements or layers may be present. In contrast, when an element or layer is referred to as being “directly on,” “directly connected to”, “directly coupled to”, or “immediately adjacent to” another element or layer, there are no intervening elements or layers present.

As used herein, the term “substantially,” “about,” and similar terms are used as terms of approximation and not as terms of degree, and are intended to account for the inherent deviations in measured or calculated values that would be recognized by those of ordinary skill in the art. Moreover, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that amending to expressly recite any such subranges would comply with the requirements of 35 U.S.C. § 112(a), and 35 U.S.C. § 132(a).

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims, and equivalents thereof.

What is claimed is:

1. A light-emitting device comprising:
a first electrode;
a second electrode; and
an interlayer between the first electrode and the second electrode and comprising an emission layer,
wherein the emission layer comprises a first host, a second host, and a dopant,
the first host and the second host are to form an exciplex, and
the exciplex, the first host, and the second host satisfy Condition 1:
0.5 eV≤[{T₁(H1)−S₁(Ex)}+{T₁(H2)−S₁(Ex)}]≤0.9 eV, and <Condition 1>
wherein, in Condition 1,
T₁(H1) indicates a lowest excitation triplet energy level of the first host,
T₁(H2) indicates a lowest excitation triplet energy level of the second host, and
S₁(Ex) indicates a lowest excitation singlet energy level of the exciplex.

2. The light-emitting device of claim 1, wherein the lowest excitation triplet energy level of the exciplex is greater than 2.7 eV.

3. The light-emitting device of claim 1, wherein the first host comprises a carbazole partial structure.

4. The light-emitting device of claim 1, wherein the first host is represented by Formula 1: 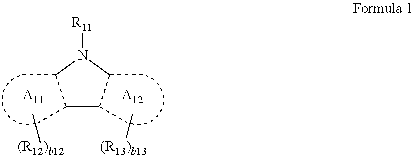
wherein, in Formula 1,
A₁₁ and A₁₂ are each independently a C₃-C₂₀ carbocyclic group or a C₁-C₂₀ heterocyclic group,
R₁₁ to R₁₃ are each independently a group represented by *-(L₁₁)ₐ₁₁-R₁₄, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂), wherein at least one of R₁₁ to R₁₃ is the group represented by *-(L₁₁)ₐ₁₁-R₁₄,
b12 and b13 are each independently an integer from 1 to 10,
L₁₁ is a C₃-C₆₀ carbocyclic group unsubstituted or substituted with at least one R₁₀ₐ or a C₁-C₆₀ heterocyclic group unsubstituted or substituted with at least one R₁₀ₐ,
a11 is an integer from 0 to 5,
R₁₄ is a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂), and
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁₁ to Q₁₃, Q₂₁ to Q₂₃, Q₃₁ to Q₃₃ and Q₃₀₁ to Q₃₀₃ are each independently hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₆₀ alkoxy group, or a C₃-C₆₀ carbocyclic group or a C₁-C₆₀ heterocyclic group that is unsubstituted or substituted with deuterium, —F, a cyano group, a C₁-C₆₀ alkyl group, a C₁-C₆₀ alkoxy group, a phenyl group, a biphenyl group, or any combination thereof, and
* indicates a binding site to a neighboring atom.

5. The light-emitting device of claim 1, wherein the first host is selected from Group I:

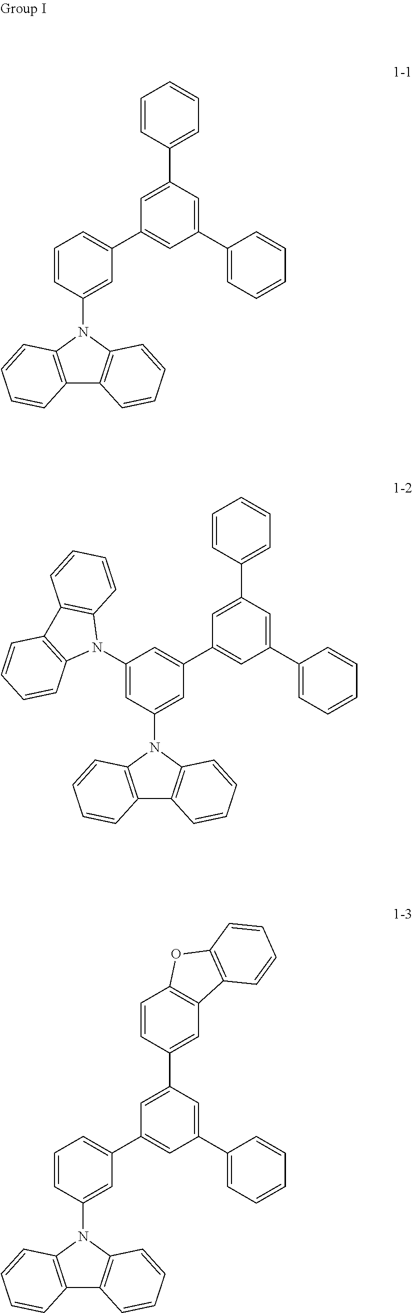 [SMILES: c1ccc(-c2cc(-c3ccccc3)cc(-c3cccc(-n4c5ccccc5c5ccccc54)c3)c2)cc1 ; c1ccc(-c2cc(-c3ccccc3)cc(-c3cc(-n4c5ccccc5c5ccccc54)cc(-n4c5ccccc5c5ccccc54)c3)c2)cc1 ; c1ccc(-c2cc(-c3cccc(-n4c5ccccc5c5ccccc54)c3)cc(-c3ccc4oc5ccccc5c4c3)c2)cc1]

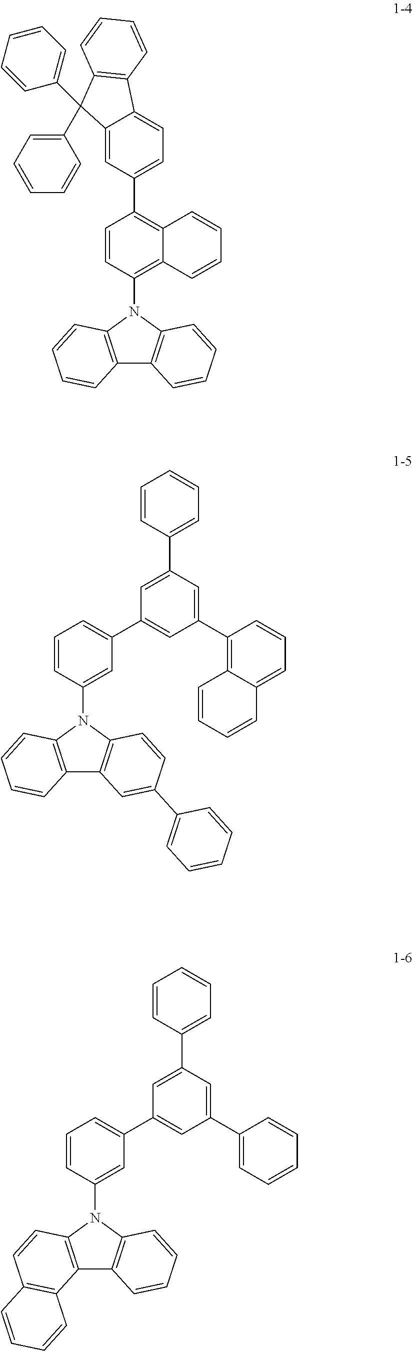 [SMILES: c1ccc(C2(c3ccccc3)c3ccccc3-c3ccc(-c4ccc(-n5c6ccccc6c6ccccc65)c5ccccc45)cc32)cc1 ; c1ccc(-c2cc(-c3cccc(-n4c5ccccc5c5cc(-c6ccccc6)ccc54)c3)cc(-c3cccc4ccccc34)c2)cc1 ; c1ccc(-c2cc(-c3ccccc3)cc(-c3cccc(-n4c5ccccc5c5c6ccccc6ccc54)c3)c2)cc1]

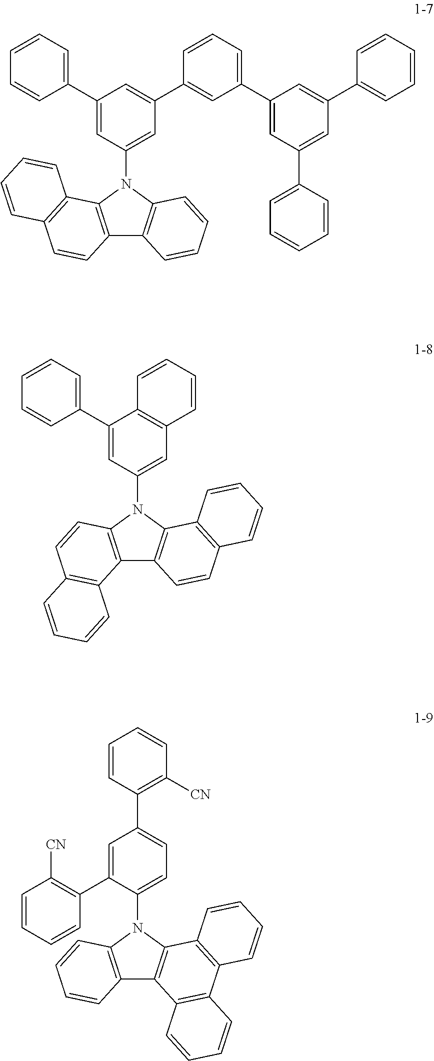 [SMILES: c1ccc(-c2cc(-c3ccccc3)cc(-c3cccc(-c4cc(-c5ccccc5)cc(-n5c6ccccc6c6ccc7ccccc7c65)c4)c3)c2)cc1 ; c1ccc(-c2cc(-n3c4ccc5ccccc5c4c4ccc5ccccc5c43)cc3ccccc23)cc1 ; N#Cc1ccccc1-c1ccc(-n2c3ccccc3c3c4ccccc4c4ccccc4c32)c(-c2ccccc2C#N)c1]

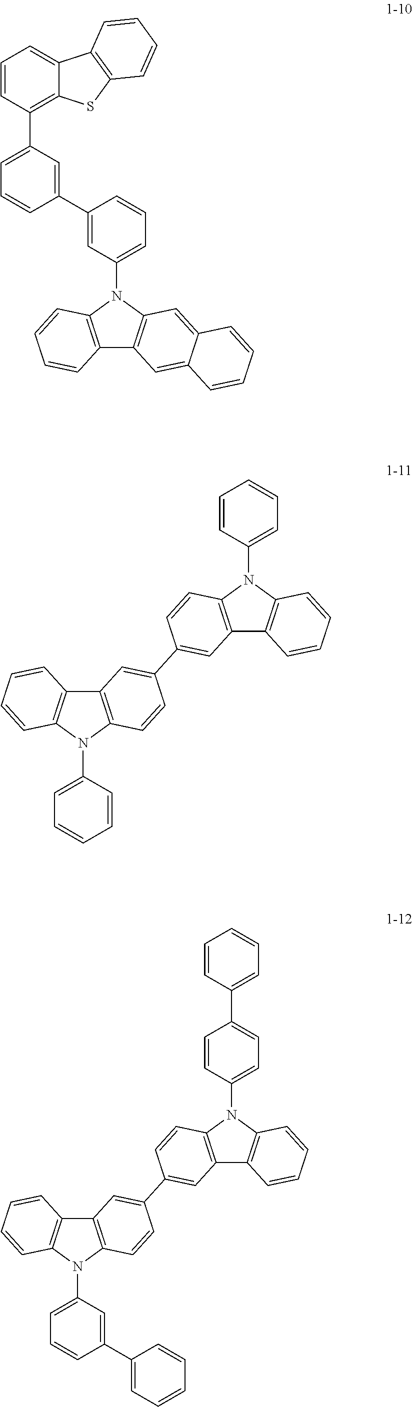 [SMILES: c1cc(-c2cccc(-n3c4ccccc4c4cc5ccccc5cc43)c2)cc(-c2cccc3c2sc2ccccc23)c1 ; c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5cccc(-c6ccccc6)c5)ccc43)cc2)cc1]

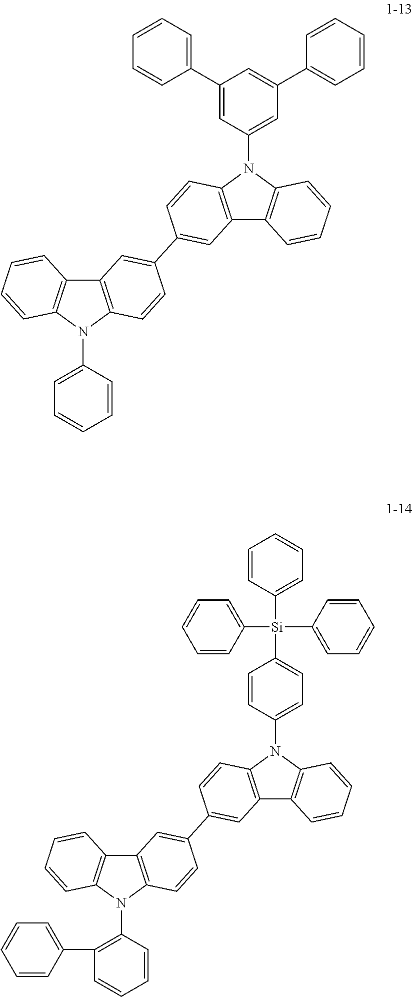 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccc([Si](c5ccccc5)(c5ccccc5)c5ccccc5)cc4)ccc32)cc1]

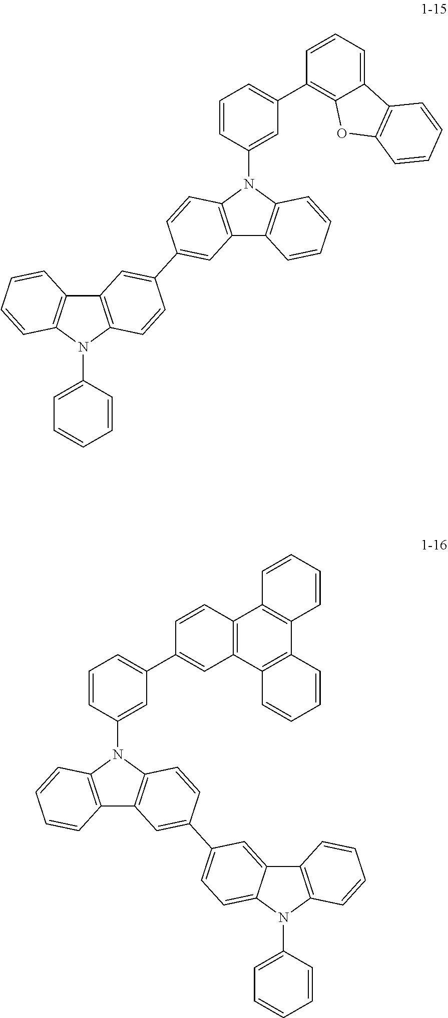 [SMILES: c1ccc(-n2c3ccccc3c3cc(-c4ccc5c(c4)c4ccccc4n5-c4cccc(-c5cccc6c5oc5ccccc56)c4)ccc32)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1]

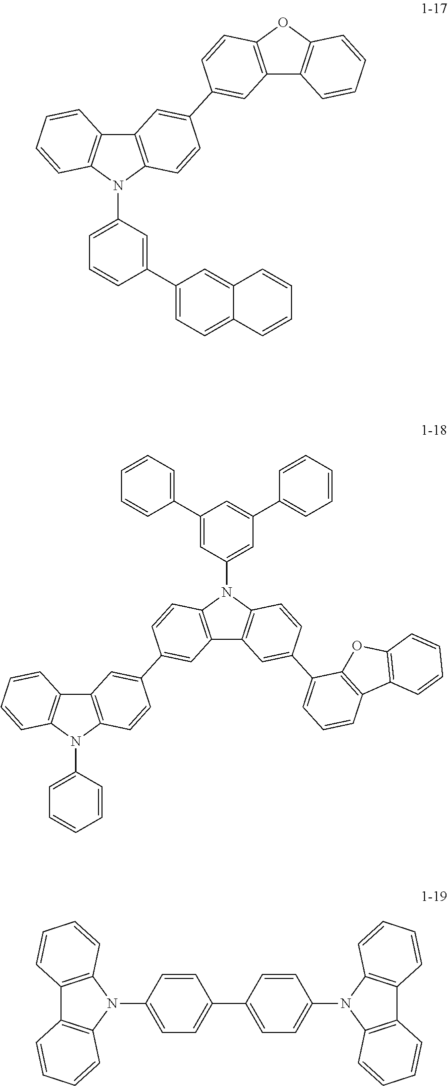 [SMILES: c1cc(-c2ccc3ccccc3c2)cc(-n2c3ccccc3c3cc(-c4ccc5oc6ccccc6c5c4)ccc32)c1 ; c1ccc(-c2cc(-c3ccccc3)cc(-n3c4ccc(-c5ccc6c(c5)c5ccccc5n6-c5ccccc5)cc4c4cc(-c5cccc6c5oc5ccccc56)ccc43)c2)cc1 ; c1ccc2c(c1)c1ccccc1n2-c1ccc(-c2ccc(-n3c4ccccc4c4ccccc43)cc2)cc1]

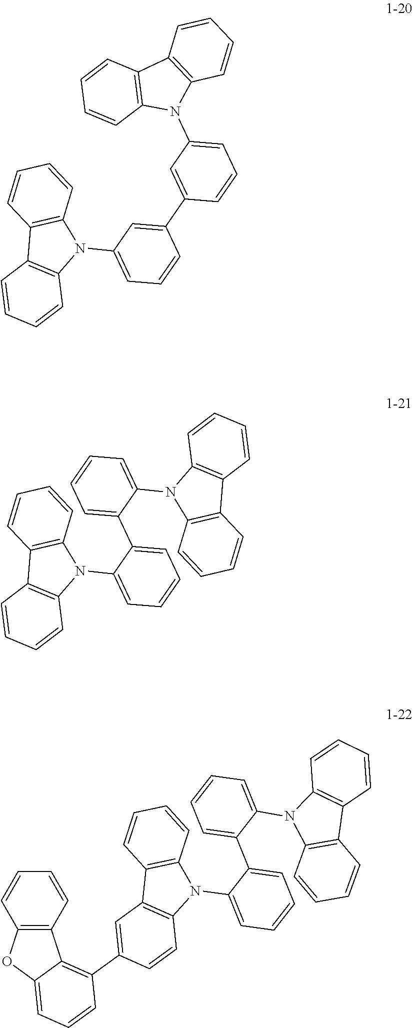 [SMILES: c1cc(-c2cccc(-n3c4ccccc4c4ccccc43)c2)cc(-n2c3ccccc3c3ccccc32)c1 ; c1ccc(-n2c3ccccc3c3ccccc32)c(-c2ccccc2-n2c3ccccc3c3ccccc32)c1 ; c1ccc(-n2c3ccccc3c3ccccc32)c(-c2ccccc2-n2c3ccccc3c3cc(-c4cccc5oc6ccccc6c45)ccc32)c1]

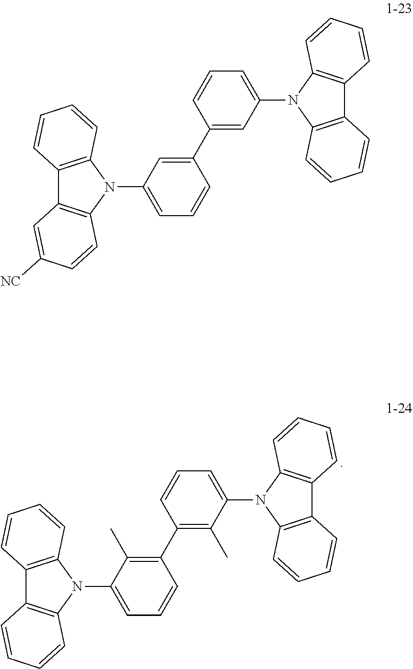 [SMILES: N#Cc1ccc2c(c1)c1ccccc1n2-c1cccc(-c2cccc(-n3c4ccccc4c4ccccc43)c2)c1 ; Cc1c(-c2cccc(-n3c4ccccc4c4ccccc43)c2C)cccc1-n1c2ccccc2c2ccccc21]

6. The light-emitting device of claim 1, wherein the second host comprises a triazine partial structure.

7. The light-emitting device of claim 1, wherein the second host is represented by Formula 2: 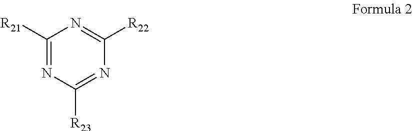
wherein, in Formula 2,
R₂₁ to R₂₃ are each independently a group represented by *-(L₂₁)ₐ₂₁-R₂₄, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂), wherein at least one of R₂₁ to R₂₃ is the group represented by *-(L₂₁)ₐ₂₁—R₂₄,
L₂₁ is a C₃-C₆₀ carbocyclic group unsubstituted or substituted with at least one R₁₀ₐ or a C₁-C₆₀ heterocyclic group unsubstituted or substituted with at least one R₁₀ₐ,
a21 is an integer from 0 to 5,
R₂₄ is a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂), and
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁₁ to Q₁₃, Q₂₁ to Q₂₃, Q₃₁ to Q₃₃ and Q₃₀₁ to Q₃₀₃ are each independently hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆₀ heterocyclic group that is unsubstituted or substituted with deuterium, —F, a cyano group, a C₁-C₆₀ alkyl group, a alkoxy group, a phenyl group, a biphenyl group, or any combination thereof, and
* indicates a binding site to a neighboring atom.

8. The light-emitting device of claim 1, wherein the second host is selected from Group II:

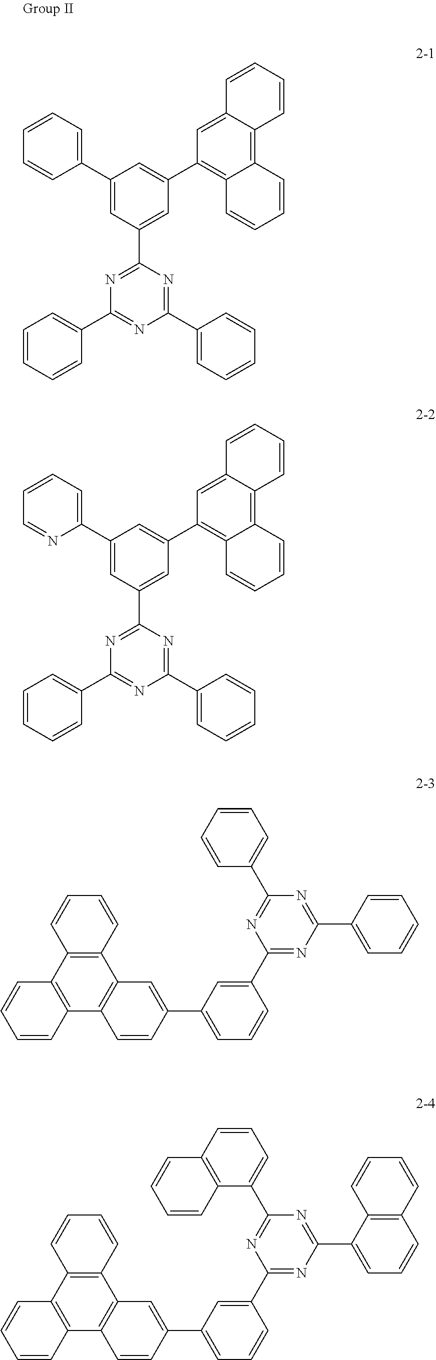 [SMILES: c1ccc(-c2cc(-c3nc(-c4ccccc4)nc(-c4ccccc4)n3)cc(-c3cc4ccccc4c4ccccc34)c2)cc1 ; c1ccc(-c2nc(-c3ccccc3)nc(-c3cc(-c4ccccn4)cc(-c4cc5ccccc5c5ccccc45)c3)n2)cc1 ; c1ccc(-c2nc(-c3ccccc3)nc(-c3cccc(-c4ccc5c6ccccc6c6ccccc6c5c4)c3)n2)cc1 ; c1cc(-c2ccc3c4ccccc4c4ccccc4c3c2)cc(-c2nc(-c3cccc4ccccc34)nc(-c3cccc4ccccc34)n2)c1]

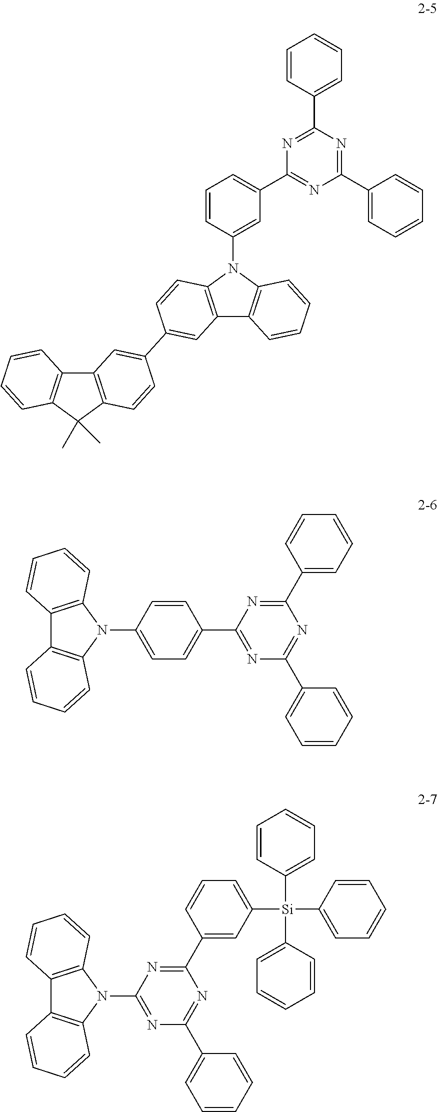 [SMILES: CC1(C)c2ccccc2-c2cc(-c3ccc4c(c3)c3ccccc3n4-c3cccc(-c4nc(-c5ccccc5)nc(-c5ccccc5)n4)c3)ccc21 ; c1ccc(-c2nc(-c3ccccc3)nc(-c3ccc(-n4c5ccccc5c5ccccc54)cc3)n2)cc1 ; c1ccc(-c2nc(-c3cccc([Si](c4ccccc4)(c4ccccc4)c4ccccc4)c3)nc(-n3c4ccccc4c4ccccc43)n2)cc1]

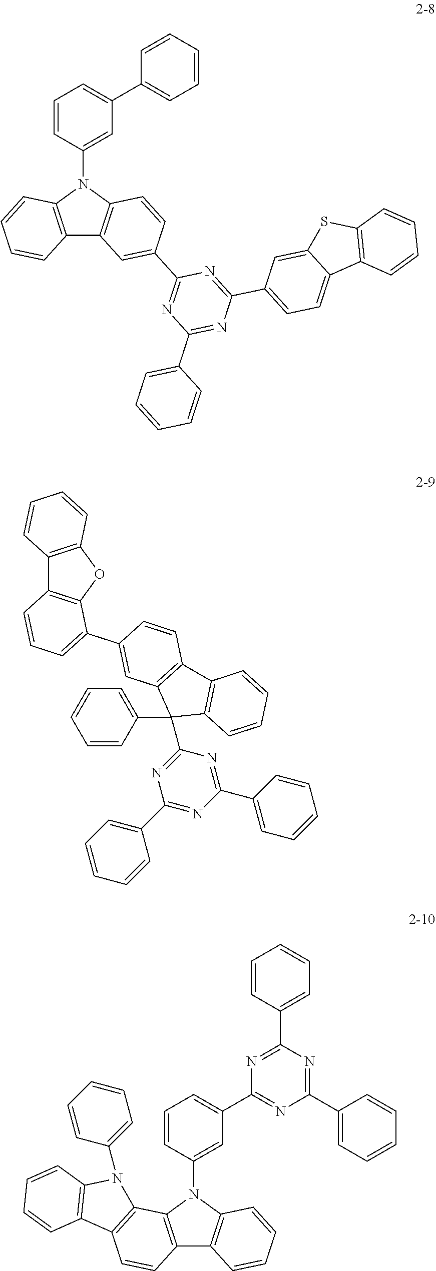 [SMILES: c1ccc(-c2cccc(-n3c4ccccc4c4cc(-c5nc(-c6ccccc6)nc(-c6ccc7c(c6)sc6ccccc67)n5)ccc43)c2)cc1 ; c1ccc(-c2nc(-c3ccccc3)nc(C3(c4ccccc4)c4ccccc4-c4ccc(-c5cccc6c5oc5ccccc56)cc43)n2)cc1 ; c1ccc(-c2nc(-c3ccccc3)nc(-c3cccc(-n4c5ccccc5c5ccc6c7ccccc7n(-c7ccccc7)c6c54)c3)n2)cc1]

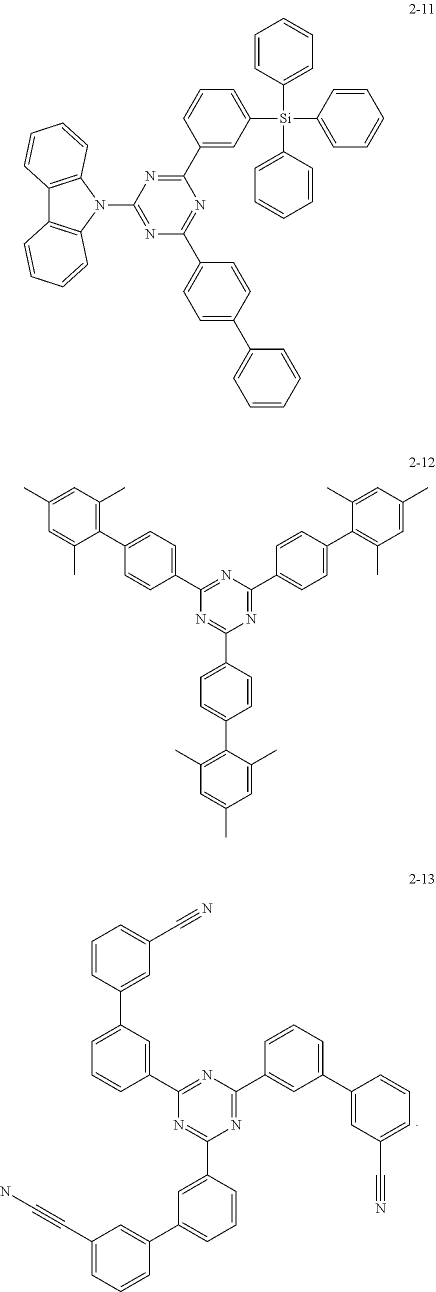

9. The light-emitting device of claim 1, wherein
the first electrode is an anode,
the second electrode is a cathode, and
the interlayer further comprises a hole transport region between the first electrode and the emission layer and an electron transport region between the emission layer and the second electrode.

10. The light-emitting device of claim 9, wherein
the hole transport region further comprises a first layer, and the first layer consists of a first organometallic compound, or
the electron transport region further comprises a second layer, and the second layer consists of a second organometallic compound, or
the hole transport region further comprises the first layer, and the electron transport region further comprises the second layer.

11. The light-emitting device of claim 10, wherein the first organometallic compound and the second organometallic compound are each independently represented by Formula 3:
M₃₁(L₃₁)ₙ₃₁(L₃₂)ₙ₃₂
wherein, in Formula 3,
M₃₁ is platinum (Pt) or iridium (Ir),
L₃₁ is a ligand represented by one of Formulae 3A to 3D,
n31 is 1 or 2,
L₃₂ is an organic ligand, and
n32 is 0, 1, 2, 3, or 4, 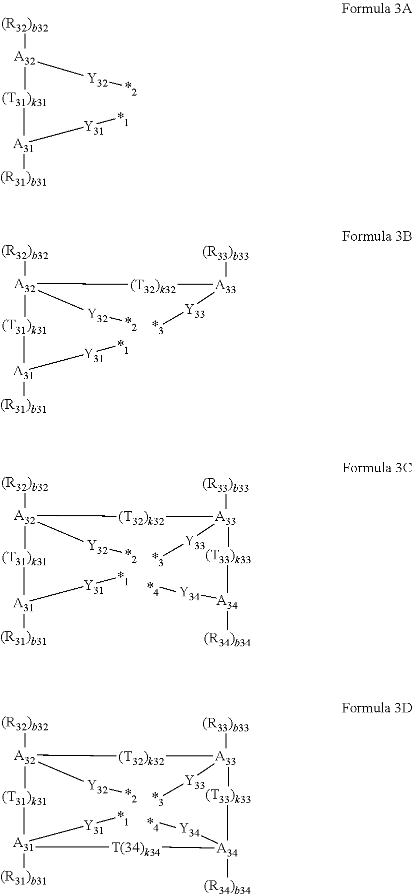
wherein, in Formulae 3A to 3D,
A₃₁ to A₃₄ are each independently a C₃-C₆₀ carbocyclic group or a C₁-C₆₀ heterocyclic group,
T₃₁ to T₃₄ are each independently a single bond, a double bond, *—O—*′, *—S—*′, *—C(═O)—*′, *—S(═O)—*′, *—C(R₃₅)(R₃₆)—*′, *—C(R₃₅)═C(R₃₆)—*′, *—C(R₃₅)=*′, *—Si(R₃₅)(R₃₆)—*′, *—B(R₃₅)—*′, *—N(R₃₅)—*′, or *—P(R₃₅)—*′,
k31 to k34 are each independently 1, 2, or 3,
Y₃₁ to Y₃₄ are each independently a single bond, *—O—*′, *—S—*′, *—C(R₃₇)(R₃₈)—′, *—Si(R₃₇)(R₃₈)—′, *—B(R₃₇)—′, *—N(R₃₇)—*′, or *—P(R₃₇)—*′,
R₃₁ to R₃₈ are each independently hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂), wherein R₃₁ to R₃₈ are optionally linked together to form a substituted or unsubstituted C₅-C₆₀ carbocyclic group or a substituted or unsubstituted C₁-C₆₀ heterocyclic group,
b31 to b34 are each independently an integer from 0 to 10, and
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁₁ to Q₁₃, Q₂₁ to Q₂₃, Q₃₁ to Q₃₃ and Q₄₀₁ to Q₄₀₃ are each independently hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₆₀ alkoxy group, or a C₃-C₆₀ carbocyclic group or a C₁-C₆₀ heterocyclic group that is unsubstituted or substituted with deuterium, —F, a cyano group, a C₁-C₆₀ alkyl group, a C₁-C₆₀ alkoxy group, a phenyl group, a biphenyl group, or any combination thereof,
* and *′ each indicate a binding site to a neighboring atom, and
*₁, *₂, *₃, and *₄ each indicate a binding site to M₃₁.

12. The light-emitting device of claim 10, wherein the organometallic compound is selected from Group III:

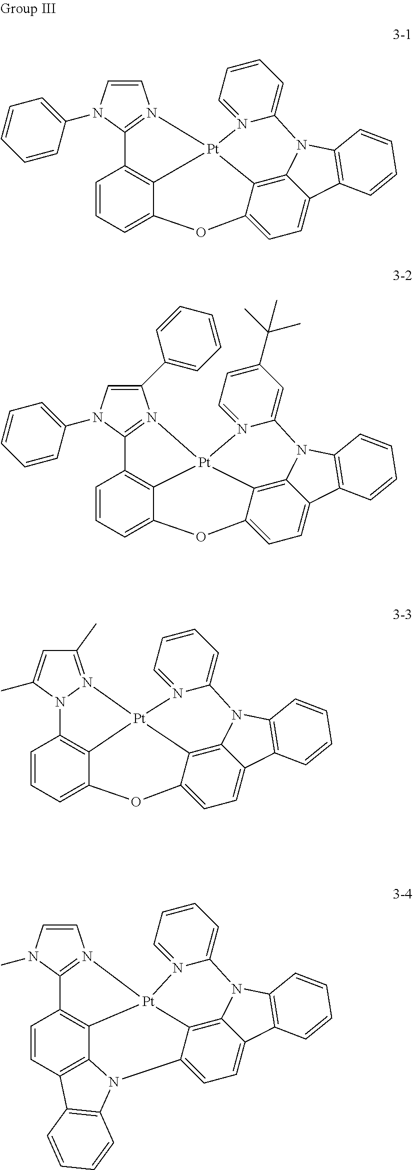

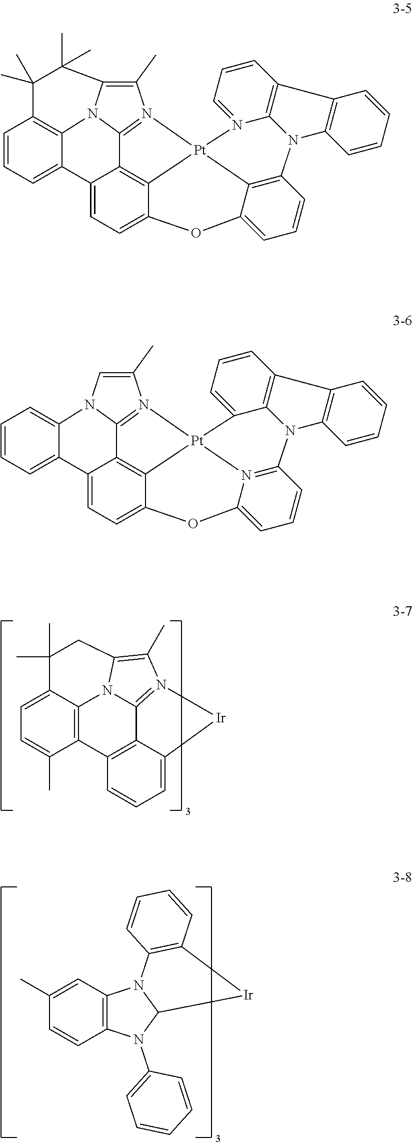

13. The light-emitting device of claim 10, wherein
the emission layer directly contacts the first layer, or
the emission layer directly contacts the second layer, or
the emission layer directly contacts each of the first layer and the second layer.

14. The light-emitting device of claim 10, wherein the hole transport region further comprises a third layer, and a difference between a highest occupied molecular orbital (HOMO) energy level of the first layer and a HOMO energy level of the third layer is equal to or less than 0.3 eV.

15. The light-emitting device of claim 14, wherein the first layer directly contacts the third layer.

16. The light-emitting device of claim 10, wherein the electron transport region further comprises a fourth layer, and a difference between a lowest unoccupied molecular orbital (LUMO) energy level of the second layer and a LUMO energy level of the fourth layer is equal to or less than 0.3 eV.

17. The light-emitting device of claim 16, wherein the second layer directly contacts the fourth layer.

18. The light-emitting device of claim 1, wherein the dopant is to emit phosphorescence, fluorescence or delayed fluorescence.

19. An electronic apparatus comprising:
the light-emitting device of claim 1; and
a thin-film transistor,
wherein the thin-film transistor comprises a source electrode and a drain electrode, and the first electrode of the light-emitting device is electrically connected to the source electrode or the drain electrode of the thin-film transistor.

20. The electronic apparatus of claim 19, wherein the electronic apparatus further comprises a color filter, a color conversion layer, a touchscreen layer, a polarization layer, or any combination thereof.
